(19) United States
(12) Patent Application Publication
(10) Pub. No.: US 2024/0110927 A1
(43) Pub. Date: Apr. 4, 2024

(54) END STAGE RENAL DISEASE BIOMARKER PANEL

(71) Applicant: Joslin Diabetes Center, Inc., Boston, MA (US)

(72) Inventor: Andrzej Krolewski, Needham, MA (US)

(21) Appl. No.: 18/163,780

(22) Filed: Feb. 2, 2023

Related U.S. Application Data
(63) Continuation of application No. PCT/US2021/044658, filed on Aug. 5, 2021.
(60) Provisional application No. 63/197,120, filed on Jun. 4, 2021.
(60) Provisional application No. 63/061,616, filed on Aug. 5, 2020.

(51) Int. Cl. G01N 33/68

(52) U.S. Cl. CPC G01N 33/6893; G01N 2800/347; G01N 2800/50; G01N 2800/52

(57) ABSTRACT

The invention provides methods (e.g., in vitro methods) for identifying subjects at risk of renal decline and/or progression to end stage renal disease (ESRD). Also included are diagnostic and prognostic tools using markers (e.g., protein biomarkers) that may be used to identify subjects who are at risk of developing ESRD.

### RELATED APPLICATIONS

This application claims priority to U.S. Provisional Application No. 63/061,616, filed on Aug. 5, 2020, and claims priority to U.S. Provisional Application No. 63/197,120, filed on Jun. 4, 2021. The contents of the foregoing priority applications are incorporated by reference herein.

### GOVERNMENT SUPPORT

This invention was made with government support under Grant No. RO1 DK041526 awarded by the National Institutes of Health/National Institute of Diabetes and Digestive and Kidney Diseases, and Grant No. DP3 DK112177 awarded by the National Institute of Diabetes and Digestive and Kidney Diseases. The government has certain rights in the invention.

### SEQUENCE LISTING

The instant application contains a Sequence Listing which has been submitted electronically in ASCII format and is hereby incorporated by reference in its entirety. Said ASCII copy, created on Aug. 4, 2021, is named J103021_1070_WO_0028_1_SL.txt and is 107,501 bytes in size.

### BACKGROUND

End stage renal disease (ESRD), also referred to as end stage kidney disease (ESKD), is the last stage of chronic kidney disease (CKD), and occurs when a person's kidneys can no longer support their body's needs. For Type I diabetes (T1D) patients in the U.S., ESRD remains the major cause of premature morbidity and mortality, typically developing decades after onset of T1D and resulting from a period of progressive renal decline (1-4). Patients with Type 2 diabetes mellitus (T2D) are at increased risk of end stage renal disease and they contribute to one-half of all new cases of ESRD in the U.S. population (5, 6), in which this devastating outcome develops as a result of progressive renal decline (7).

Despite significant efforts to understand the mechanism of progressive renal decline and ESRD in diabetic patients with impaired renal function, very little is known about these mechanisms. Further, the incidence of ESRD in diabetes patients continues to increase despite improvements in glycemic control and advances in reno-protective therapies, which are almost universally implemented. Given the clear limitations of current therapies and the limited sensitivity and low predictive value of currently known clinical characteristics, accurate diagnosis and assessment of the risk of renal decline and the risk of developing ESRD in diabetic patients remains a challenge. As such, there remains a need for methods, compositions and systems for identifying, diagnosing and treating patients with, or suspected of having, renal decline and/or ESRD, in an effort to prevent the progression of this devastating disease.

### SUMMARY OF THE INVENTION

The present disclosure is based, at least in part, on the discovery of an association between certain biomarkers (e.g., protein biomarkers) and a subject having, or at risk of developing, renal decline (RD) and/or end stage renal disease (ESRD; ESRD is also referred to as end stage kidney disease (ESKD)). Accordingly, the present disclosure provides that certain factors disclosed herein can be used, e.g., as biomarkers, to predict the risk of developing renal decline and/or the risk of developing (or progressing to) ESRD in a subject (e.g., a subject having a disorder associated with chronic kidney failure, such as diabetes). The present disclosure also provides methods, compositions, and systems for detecting one or more RD-associated proteins (e.g., any one of TNF-R1, TNF-R2, CD27, LTBR, TNF-RSF6B, TNF-RSF10A, TNF-RSF4, EDA2R, RELT, CD160, IL-1RT1, DLL1, LAYN, MMP7, NBL1, PI3, WFDC2, EFNA4, EPHA2, GFR-alpha-1, and KIM1) and/or ESRD-associated proteins (e.g., any one of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, and KIM1) in a sample from a subject with, or suspected of having, renal decline (RD) or end stage renal disease (ESRD). Further, the present disclosure provides methods, compositions and systems for identifying subjects with renal decline (e.g., early progressive renal decline (RD^{early}) and/or late progressive renal decline (RD^{late})) and/or end stage renal disease. The present disclosure also provides methods, compositions and systems for identifying subjects with early progressive renal decline who are at risk of progressing to end Stage Renal Disease (ESRD). The present disclosure also provides methods, compositions and systems for monitoring efficacy of a renal decline or an end-stage renal disease (ESRD) treatment regimen in a human subject. The methods, compositions and systems provided herein are also useful for the development of accurate algorithms (with high sensitivity and high positive predictive value) for predicting and/or diagnosing subjects (e.g., subjects with T1D or T2D) with renal decline and for predicting and/or diagnosing the probability of progressing to ESRD during 5 years of observation.

Accordingly, in one aspect, the disclosure provides a method for determining whether a human subject has or is at risk of developing renal decline, comprising detecting the level of at least one renal decline marker in a biological sample from the human subject, wherein the human subject has or is at risk of developing renal decline if the level of the at least one renal decline marker correlates with a known standard for a human subject who has or is at risk of developing renal decline, or wherein the human subject does not have or is not at risk of developing renal decline if the level of the at least one renal decline marker correlates with a known standard for a human subject who does not have or is not at risk of developing renal decline.

In one embodiment, the method further comprises comparing the level of the at least one renal decline marker from the biological sample from the human subject to a non-renal-decliner control level of the at least one renal decline marker; and determining whether the level of the at least one renal decline marker from the biological sample is equal to or higher than the level of the at least one renal decline marker of a non-renal-decliner control, wherein a higher level of the at least one renal decline marker from the biological sample from the human subject relative to the level of the at least one renal decline marker from the non-renal-decliner control indicates that the human subject has or is at risk of developing renal decline.

In one embodiment, the method further comprises comparing the level of the at least one renal decline marker from the biological sample from the human subject to a normoalbuminuric control level of the at least one renal decline marker; and determining whether the level of the at least one renal decline marker from the biological sample is equal to or higher than the level of the at least one renal decline marker of a normoalbuminuric control, wherein a higher level of the at least one renal decline marker from the biological sample from the human subject relative to the level of the at least one renal decline marker from the normoalbuminuric control indicates that the human subject has or is at risk of developing renal decline.

In another embodiment, the method further comprises contacting the biological sample from the human subject with a device for measuring the protein level of the at least one renal decline marker. In one embodiment, the device is capable of performing an immunoassay, a mass spectrometry analysis, a Slow Off-rate Modified Aptamer (SOMA)scan platform analysis or an OLINK Proximity Extension Assay based proteomic platform analysis. In one embodiment, the at least one renal decline marker includes at least one of TNF-R1, TNF-R2, CD27, LTBR, TNF-RSF6B, TNF-RSF10A, TNF-RSF4, EDA2R, RELT, CD160, IL-1RT1, DLL1, LAYN, MMP7, NBL1, PI3, WFDC2, EFNA4, EPHA2, GFR-alpha-1, and KIM1.

In one embodiment, the method further comprises measuring an estimated glomerular function rate (eGFR) slope of the human subject and determining whether the eGFR slope of the human subject indicates that the human subject has or is at risk of developing renal decline. In yet another embodiment, an eGFR slope of at least <−5 ml/min/year indicates that the human subject has or is at risk of developing renal decline. In one embodiment, an eGFR slope of at least <−10 ml/min/year indicates that the human subject has or is at risk of developing renal decline. In one embodiment, an eGFR slope of at least <−15 ml/min/year indicates that the human subject has or is at risk of developing renal decline. In one embodiment, the renal decline is (i) a very fast renal decline comprising an estimated time to reach onset of end-stage renal disease (ESRD) of 2-6 years, (ii) a fast renal decline comprising an estimated time to reach onset of end-stage renal disease (ESRD) of 6-10 years, or (iii) a moderate renal decline comprising an estimated time to reach onset of end-stage renal disease (ESRD) of 10-20 years.

In one embodiment, the method further comprises measuring a urine albumin to creatinine ratio (ACR) of the human subject, and determining whether the ACR of the human subject indicates that the human subject has micro-albuminuria or macro-albuminuria. In one embodiment, the human subject has early progressive renal decline or late progressive renal decline. In another embodiment, the human subject has type I diabetes (T1D) or type 2 diabetes (T2D). In one embodiment, the renal decline is early renal decline. In one embodiment, the renal decline is late renal decline.

In one embodiment, the method further comprises treating the human subject having or at risk of developing renal decline.

In another aspect, the disclosure provides a method for determining whether a human subject has or is at risk of developing end-stage renal disease (ESRD), comprising detecting the level of at least one ESRD marker in a biological sample from the human subject, wherein the human subject has or is at risk of developing ESRD if the level of the at least one ESRD marker correlates with a known standard for a human subject who has or is at risk of developing ESRD, or wherein the human subject does not have or is not at risk of developing ESRD if the level of the at least one ESRD marker correlates with a known standard for a human subject who does not have or is not at risk of developing ESRD.

In one embodiment, the method further comprises comparing the level of the at least one ESRD marker from the biological sample from the human subject to a non-ESRD control level of the at least one ESRD marker; and determining whether the level of the at least one ESRD marker from the biological sample is equal to or higher than the level of the at least one ESRD marker of a non-ESRD control, wherein a higher level of the at least one ESRD marker from the biological sample from the human subject relative to the level of the at least one ESRD marker from the non-ESRD control indicates that the human subject has or is at risk of developing ESRD.

In one embodiment, the method further comprises comparing the level of the at least one ESRD marker from the biological sample from the human subject to a normoalbuminuric control level of the at least one ESRD marker; and determining whether the level of the at least one ESRD marker from the biological sample is equal to or higher than the level of the at least one ESRD marker of a normoalbuminuric control, wherein a higher level of the at least one ESRD marker from the biological sample from the human subject relative to the level from the normoalbuminuric control indicates that the human subject has or is at risk of developing ESRD.

In one embodiment, the method further comprises contacting the biological sample from the human subject with a device for measuring the level of the at least one ESRD marker. In one embodiment, the device is useful for performing an immunoassay, a mass spectrometry analysis, a Slow Off-rate Modified Aptamer (SOMA)scan platform analysis or an OLINK Proximity Extension Assay based proteomic platform analysis. In another embodiment, the at least one ESRD marker includes at least one of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, and KIM1.

In one embodiment, the method further comprises measuring a urine albumin to creatinine ratio (ACR) of the human subject, and determining whether the ACR of the human subject indicates that the human subject has micro-albuminuria or macro-albuminuria.

In one embodiment, the method further comprises determining a baseline renal function of the human subject.

In one embodiment, the method further comprises measuring an estimated glomerular function rate (eGFR) slope of the human subject and determining a time to onset of ESRD for the human subject using the level of the at least one ESRD marker and/or the eGFR slope of the human subject. In one embodiment, an eGFR slope of at least <−15 ml/min/year indicates that the time to onset of ESRD for the human subject is 2-6 years. In another embodiment an eGFR slope of between <−15 ml/min/year and <−10 ml/min/year indicates that the time to onset of ESRD for the human subject is 6-10 years. In one embodiment, an eGFR slope of between <−10 ml/min/year and <−5 ml/min/year indicates that the time to onset of ESRD for the human subject is 10-20 years. In one embodiment, the time to reach onset of ESRD for the human subject is 2-6 years, 6-10 years, or 10-20 years. In one embodiment, the human subject has early progressive renal decline or late progressive renal decline. In one embodiment, the human subject has type I diabetes (T1D) or type 2 diabetes (T2D).

In one embodiment, the method further comprises treating the human subject having or at risk of developing ESRD. In one embodiment, baseline albuminuria and/or baseline GFR are determined.

In another aspect, the disclosure provides a method of monitoring the progression of renal decline or end-stage renal disease (ESRD) in a human subject, comprising contacting a biological sample from the human subject with a device for assaying the level of at least one renal decline marker or at least one ESRD marker selected from one or more of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22, measuring the amount of the at least one renal decline marker or the at least one ESRD marker in the biological sample as compared to a control sample, wherein an increased or a decreased level of the at least one renal decline marker or the at least one ESRD marker relative to the control sample indicates progression of renal decline or ESRD in the human subject. In one embodiment, the device is useful for performing an immunoassay, a mass spectrometry analysis, a Slow Off-rate Modified Aptamer (SOMA)scan platform analysis or an OLINK Proximity Extension Assay based proteomic platform analysis.

In yet another aspect, the disclosure provides a method of monitoring efficacy of a renal decline or an end-stage renal disease (ESRD) treatment regimen in a human subject, comprising obtaining a first biological sample from the human subject at a first time point; administering the treatment regimen to the human subject; obtaining a second biological sample from the human subject at a second time point; detecting at least one protein level selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22 in the first sample; and detecting the at least one protein level in the second sample.

In one embodiment, the method further comprises changing or repeating the treatment regimen when the at least one protein level for the first sample is the same as the at least one protein level for the second sample.

In one embodiment, the method further comprises discontinuing the treatment regimen when the at least one protein level of the second sample is the same as the level corresponding to a healthy individual. In one embodiment, the detecting is performed with a device for measuring the level of the at least one ESRD marker. In one embodiment, the device is useful for performing an immunoassay, a mass spectrometry analysis, a Slow Off-rate Modified Aptamer (SOMA) scan platform analysis or an OLINK Proximity Extension Assay based proteomic platform analysis. In one embodiment, the biological sample is selected from the group consisting of a blood sample, a plasma sample, a serum sample, a saliva sample and a urine sample.

In another aspect, the disclosure provides a method of determining the approximate risk of renal decline (RD) or end-stage renal disease (ESRD) in a human subject, comprising a) detecting, in a biological sample from the human subject, the level of at least two RD-associated proteins of a biomarker panel or at least two ESRD-associated proteins of a biomarker panel, wherein the biomarker panel comprises at least two proteins selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22; and b) determining the approximate risk of renal decline (RD) for the human subject, and/or the risk of end-stage renal disease (ESRD) of the human subject. In one embodiment, the determining comprises employing an algorithm to generate a renal decline (RD) risk score or end-stage renal disease (ESRD) risk score, wherein the algorithm performs operations comprising i) adjusting each RD-associated protein level or each ESRD-associated protein level by a predetermined coefficient to generate an adjusted value, and ii) adding or multiplying the adjusted value together, thereby generating the RD risk score or the ESRD risk score. In one embodiment, the level of the at least two least two RD-associated proteins or the at least two least two ESRD-associated proteins are assessed by an immunoassay, a Slow Off-rate Modified Aptamer (SOMA) scan platform, or an OLINK Proximity Extension Assay based proteomic platform. In one embodiment, the algorithm performs operations further comprising i) determining an albumin-to-creatinine ratio (ACR) for the human subject; ii) adjusting the ACR by a predetermined coefficient to generate an adjusted ACR value; and iii) adding or multiplying the adjusted values together, thereby generating the RD risk score or the ESRD risk score. In one embodiment, the algorithm performs operations further comprising i) determining a systolic blood pressure (SBP) for the human subject; ii) adjusting the SBP by a predetermined coefficient to a generate an adjusted SBP value; and iii) adding or multiplying the adjusted values together, thereby generating the RD risk score or the ESRD risk score. In one embodiment, the algorithm performs operations further comprising i) determining an estimated glomerular filtration rate (eGFR) for the human subject; ii) adjusting the eGFR by a predetermined coefficient to a generate an adjusted eGFR value; and iii) adding or multiplying the adjusted values together, thereby generating the RD risk score or the ESRD risk score.

In one embodiment, the method further comprises c) generating a report that provides the RD risk score and/or the ESRD risk score. In one embodiment, the biological sample is selected from the group consisting of a blood sample, a plasma sample, a serum sample, a saliva sample and a urine sample.

In yet another aspect, the disclosure provides a protein array comprising at least two biomarkers useful for diagnosing, predicting, and/or monitoring of renal decline or end-stage renal disease in a sample of a human subject, wherein the biomarkers are selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22, or fragments, or variants thereof.

In yet another aspect, the disclosure provides a test panel comprising the protein array of the disclosure.

In yet another aspect, the disclosure provides a kit or assay device comprising the test panel of the disclosure.

In one aspect, the disclosure provides a method for determining whether a human subject has renal decline, comprising detecting the level of at least one renal decline marker in a biological sample from the human subject, wherein the human subject has or is at risk of developing renal decline if the level of the at least one renal decline marker correlates with a known standard for a human subject who has or is at risk of developing renal decline, or wherein the human subject does not have or is not at risk of developing renal decline if the level of the at least one renal decline marker correlates with a known standard for a human subject who does not have or is not at risk of developing renal decline.

In another aspect, the disclosure provides a method for determining whether a human subject is at risk of developing renal decline, comprising detecting the level of at least one renal decline marker in a biological sample from the human subject, wherein the human subject has or is at risk of developing renal decline if the level of the at least one renal decline marker correlates with a known standard for a human subject who has or is at risk of developing renal decline, or wherein the human subject does not have or is not at risk of developing renal decline if the level of the at least one renal decline marker correlates with a known standard for a human subject who does not have or is not at risk of developing renal decline.

In one embodiment, the method further comprises comparing the level of the at least one renal decline marker from the biological sample from the human subject to a non-renal-decliner control level of the at least one renal decline marker; and determining whether the level of the at least one renal decline marker from the biological sample is equal to or higher than the level of the at least one renal decline marker of a non-renal-decliner control, wherein a higher level of the at least one renal decline marker from the biological sample from the human subject relative to the level of the at least one renal decline marker from the non-renal-decliner control indicates that the human subject has or is at risk of developing renal decline.

In one embodiment, the method further comprises comparing the level of the at least one renal decline marker from the biological sample from the human subject to a normoalbuminuric control level of the at least one renal decline marker; and determining whether the level of the at least one renal decline marker from the biological sample is equal to or higher than the level of the at least one renal decline marker of a normoalbuminuric control, wherein a higher level of the at least one renal decline marker from the biological sample from the human subject relative to the level of the at least one renal decline marker from the normoalbuminuric control indicates that the human subject has or is at risk of developing renal decline.

In another embodiment, the method further comprises contacting the biological sample from the human subject with a device for measuring the level of the at least one renal decline marker. In one embodiment, the device is capable of performing an immunoassay, a mass spectrometry analysis, a Slow Off-rate Modified Aptamer (SOMA)scan platform analysis or an OLINK Proximity Extension Assay based proteomic platform analysis. In one embodiment, the at least one renal decline marker includes at least one of TNF-R1, TNF-R2, CD27, LTBR, TNF-RSF6B, TNF-RSF10A, TNF-RSF4, EDA2R, RELT, CD160, IL-1RT1, DLL1, LAYN, MMP7, NBL1, PI3, WFDC2, EFNA4, EPHA2, GFR-alpha-1, and KIM1.

In one embodiment, the method further comprises measuring an estimated glomerular function rate (eGFR) slope of the human subject and determining whether the eGFR slope of the human subject indicates that the human subject has or is at risk of developing renal decline. In yet another embodiment, an eGFR slope of at least <−5 ml/min/year indicates that the human subject has or is at risk of developing renal decline. In one embodiment, an eGFR slope of at least <−10 ml/min/year indicates that the human subject has or is at risk of developing renal decline. In one embodiment, an eGFR slope of at least <−15 ml/min/year indicates that the human subject has or is at risk of developing renal decline. In one embodiment, the renal decline is (i) a very fast renal decline comprising an estimated time to reach onset of end-stage renal disease (ESRD) of 2-6 years, (ii) a fast renal decline comprising an estimated time to reach onset of end-stage renal disease (ESRD) of 6-10 years, or (iii) a moderate renal decline comprising an estimated time to reach onset of end-stage renal disease (ESRD) of 10-20 years.

In one embodiment, the method further comprises measuring a urine albumin to creatinine ratio (ACR) of the human subject, and determining whether the ACR of the human subject indicates that the human subject has micro-albuminuria or macro-albuminuria. In one embodiment, the human subject has early progressive renal decline or late progressive renal decline. In another embodiment, the human subject has type I diabetes (T1D) or type 2 diabetes (T2D). In one embodiment, the renal decline is early renal decline. In one embodiment, the renal decline is late renal decline.

In one embodiment, the method further comprises treating the human subject having or at risk of developing renal decline.

In another aspect, the disclosure provides a method for determining whether a human subject has end-stage renal disease (ESRD), comprising detecting the level of at least one ESRD marker in a biological sample from the human subject, wherein the human subject has or is at risk of developing ESRD if the level of the at least one ESRD marker correlates with a known standard for a human subject who has or is at risk of developing ESRD, or wherein the human subject does not have or is not at risk of developing ESRD if the level of the at least one ESRD marker correlates with a known standard for a human subject who does not have or is not at risk of developing ESRD.

In another aspect, the disclosure provides a method for determining whether a human subject is at risk of developing end-stage renal disease (ESRD), comprising detecting the level of at least one ESRD marker in a biological sample from the human subject, wherein the human subject has or is at risk of developing ESRD if the level of the at least one ESRD marker correlates with a known standard for a human subject who has or is at risk of developing ESRD, or wherein the human subject does not have or is not at risk of developing ESRD if the level of the at least one ESRD marker correlates with a known standard for a human subject who does not have or is not at risk of developing ESRD.

In one embodiment, the method further comprises comparing the level of the at least one ESRD marker from the biological sample from the human subject to a non-ESRD control level of the at least one ESRD marker; and determining whether the level of the at least one ESRD marker from the biological sample is equal to or higher than the level of the at least one ESRD marker of a non-ESRD control, wherein a higher level of the at least one ESRD marker from the biological sample from the human subject relative to the level of the at least one ESRD marker from the non-ESRD control indicates that the human subject has or is at risk of developing ESRD.

In one embodiment, the method further comprises comparing the level of the at least one ESRD marker from the biological sample from the human subject to a normoalbuminuric control level of the at least one ESRD marker; and determining whether the level of the at least one ESRD marker from the biological sample is equal to or higher than the level of the at least one ESRD marker of a normoalbuminuric control, wherein a higher level of the at least one ESRD marker from the biological sample from the human subject relative to the level from the normoalbuminuric control indicates that the human subject has or is at risk of developing ESRD.

In one embodiment, the method further comprises contacting the biological sample from the human subject with a device for measuring the level of the at least one ESRD marker. In one embodiment, the device is useful for performing an immunoassay, a mass spectrometry analysis, a Slow Off-rate Modified Aptamer (SOMA)scan platform analysis or an OLINK Proximity Extension Assay based proteomic platform analysis. In another embodiment, the at least one ESRD marker includes at least one of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, and KIM1.

In one embodiment, the method further comprises measuring a urine albumin to creatinine ratio (ACR) of the human subject, and determining whether the ACR of the human subject indicates that the human subject has micro-albuminuria or macro-albuminuria.

In one embodiment, the method further comprises determining a baseline renal function of the human subject.

In one embodiment, the method further comprises measuring an estimated glomerular function rate (eGFR) slope of the human subject and determining a time to onset of ESRD for the human subject using the level of the at least one ESRD marker and/or the eGFR slope of the human subject. In one embodiment, an eGFR slope of at least <−15 ml/min/year indicates that the time to onset of ESRD for the human subject is 2-6 years. In another embodiment an eGFR slope of between <−15 ml/min/year and <−10 ml/min/year indicates that the time to onset of ESRD for the human subject is 6-10 years. In one embodiment, an eGFR slope of between <−10 ml/min/year and <−5 ml/min/year indicates that the time to onset of ESRD for the human subject is 10-20 years. In one embodiment, the time to reach onset of ESRD for the human subject is 2-6 years, 6-10 years, or 10-20 years. In one embodiment, the human subject has early progressive renal decline or late progressive renal decline. In one embodiment, the human subject has type I diabetes (T1D) or type 2 diabetes (T2D). In one embodiment, the method further comprises treating the human subject having or at risk of developing ESRD. In one embodiment, baseline albuminuria and/or baseline GFR are determined.

In another aspect, the present disclosure provides a method of determining the approximate risk of renal decline in a human subject in a defined time period, the method comprising: a) obtaining a biological sample from the human subject; b) detecting the level of at least one ESRD protein in the biological sample, wherein the at least one ESRD protein is selected from the group consisting of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, KIM1, or combinations thereof; c) combining data on the level of the ESRD proteins with clinical data features of the human subject (such as eGFR, uACR, Clinical Chemistry laboratory measurements, vital signs, patient demographics) and d) determining the approximate risk of renal decline (RD) for the human subject as determined using a machine-learned or statistically modelled, prognostic risk-score algorithm (e.g., KidneyIntelX test platform).

### BRIEF DESCRIPTION OF THE DRAWINGS

FIGS. 1A and 1B are a graphical depiction of estimated glomerular filtration rate (eGFR_{cys}) trajectories in subjects (n=89) who developed new onset microalbuminuria (MA) and who were followed for 8-12 years. FIG. 1A shows subjects (n=65) without renal decline (i.e., non-decliners). FIG. 1B shows subjects (n=24) with progressive renal decline (i.e., decliners). The area between 0 and 2 years of follow-up indicates onset of MA (see “MA onset” in FIGS. 1A and 1B). The numbers of subjects with normoalbuminuria (NA), microalbuminuria (MA) and proteinuria (Prot) at most recent follow-up examination are shown. “E” indicates ESRD; upper shaded circular area (FIG. 1B) indicates early renal decline (RD^{early}); lower shaded circular area (FIG. 1B) indicates late renal decline (RD^{late}); eGFR_{cys} based on serum cystatin determinations.

FIG. 2 shows the histogram distribution of eGFR slopes in 364 patients in the Joslin ESRD cohort, with insert showing trajectory of renal decline in a subjects with the steepest slope (indicated by an arrow).

FIG. 3 shows distributions of fast renal decline (%) according to quartiles of serum WFDC2 and serum MMP-7.

FIG. 4A-FIG. 4C depict the profile of circulating AGP proteins and their associations with risk of progression to ESRD during 10-year follow-up. FIG. 4A shows among 127 AGP proteins, 42 were measured by the SOMAscan platform; in Cox regression analysis, the baseline plasma concentration of 6 AGP proteins were associated with risk of ESKD in each of the four cohorts. FIG. 4B graphically depicts Hazard Ratios (HRs) with 95% CIs for time to onset of ESKD according to baseline plasma levels of the 6 AGP proteins in the combined four cohorts (n=745). Estimates are per one-quartile increase (discrete variable) in plasma level of the AGP proteins after adjusting for clinical covariates important for the etiologic model, i.e., sex, duration of diabetes, systolic BP, baseline HbA1c, and eGFR, with variable stratification by study cohort. P values for comparison of quartile 4 (Q4) versus Q1 are shown (see Table 13). For distribution of values for the 6 AGP proteins, see FIG. 7A-7F. FIG. 4C shows binding preferences for Ephrin and Netrin ligands and receptors. Thin arrows indicate selective binding of one ligand with one receptor. Thick arrows represent multiple ligands binding to multiple receptors. Of the 14 AGP proteins available on the SOMAscan platform (asterisks), 6 were elevated (indicated with arrow) in subjects at risk of ESKD in the study cohorts. FLRT-2/3, fibronectin and leucine rich transmembrane protein 2 and 3; RGM-A, repulsive guidance molecule-A. **P<10-7, ***p<10-11, ****P<10-15.

FIG. 5 shows pictures of tissue expression of AGP proteins Immunofluorescence staining for EFNA4, EPHA2, and colocalization of the two AGP proteins in tissue from of kidney biopsy tissue in a nondiabetic, age-matched control (left column), a Pima subject with T2D (middle column) and low serum AGP proteins (EFNA4, 2150 relative fluorescent units [RFU]; EPHA2, 3453 RFU; and VvMes, 12.3%), and a Pima subject with T2D (right column) and high serum AGP proteins (EFNA4, 2333 RFU; EPHA2, 4462 RFU; and VvMes, 27.2%).

FIGS. 6A and 6B depict the effects of intensive glycemic control and losartan (Ln) treatment on serum levels of AGP proteins in two clinical trials. FIG. 6A shows comparison of serum EFNA4 and EPHA2 levels between subjects with standard (n=100) versus intensive glycemic control (n=100) in the ACCORD study. Standard (Std) glycemic control and intensive (Int) glycemic control measures are shown. FIG. 6B shows comparison of serum EFNA4 and EPHA2 levels between subjects with losartan treatment (second or fourth bar indicated as (+); n=37) versus placebo (first or third bar indicated as (−); n=47) in T2D Pima cohort. Data are shown as means±SEMs adjusting for sex, duration of diabetes, systolic BP, baseline HbA1c, and GFR. *P<0.05.

FIGS. 7A-7F depicts the distributions of levels of 6 AGP proteins in the 4 cohorts. The samples were diluted with three serial dilutions (40%, 1% and 0.005%) and measured by SOMAscan platform. According to the range of levels of proteins, levels with 40% dilution (EFNA4, EFNA5 and EPHA2) and 1% dilution (EPHB2, EPHB6 and UNC5C) in relative fluorescence unit (RFU) are shown. FIG. 7A depicts the distribution of EFNA4 level in the 4 cohorts; FIG. 7B depicts the distribution of EFNA5 level in the 4 cohorts; FIG. 7C depicts the distribution of EPHA2 level in the 4 cohorts; FIG. 7D depicts the distribution of EPHB2 level in the 4 cohorts; FIG. 7E depicts the distribution of EPHB6 level in the 4 cohorts; FIG. 7F depicts the distribution of UNC5C level in the 4 cohorts.

### DETAILED DESCRIPTION

### I. Definitions

Prior to setting forth the invention in detail, definitions of certain terms to be used herein are provided. Unless defined otherwise, all technical and scientific terms used herein have the same meaning as is commonly understood by one of skill in the art.

As used herein, the term “a disorder associated with chronic kidney disease” refers to a disease or condition associated with impaired kidney function which can cause kidney damage over time. Examples of disorders associated with chronic kidney disease include, but are not limited to, type 1 diabetes (T1D), type 2 diabetes (T2D), high blood pressure (HBP), glomerulonephritis, interstitial nephritis, polycystic kidney disease, prolonged obstruction of the urinary tract (e.g., from conditions such as enlarged prostate, kidney stones and some cancers), vesicoureteral reflux, and recurrent kidney infection. Chronic kidney disease and its stages (CKD 1-5) can usually be characterized or classified accordingly, such as based on the presence of either kidney damage (albuminuria) or impaired estimated glomerular filtration rate (eGFR<60 [ml/min/1.73 m²], with or without kidney damage).

As used herein, the term “comparable level” refers to a level of a biomarker that is substantially similar to the level of another, e.g., a control level. In one embodiment, two biomarkers have a comparable level if the level of the biomarker is within one standard deviation of the control biomarker level. In another embodiment, two biomarkers have a comparable level if the level of the biomarker is 20% or less of the level of the control biomarker level. In another embodiment, two biomarkers have a comparable level if the level of the biomarker is 15% or less of the level of the control biomarker level. In another embodiment, two biomarkers have a comparable level if the level of the biomarker is 10% or less of the level of the control biomarker level. In another embodiment, two biomarkers have a comparable level if the level of the biomarker is 5% or less of the level of the control biomarker level.

As used herein, the term “decliner biomarker” refers to a molecule that is associated with renal decline (or with increased risk of renal decline) in a subject. In one embodiment, an increase in the level of a decliner biomarker in a subject relative to a non-decliner level (or a normoalbuminuric control level, or a healthy control level, or a standard control level) indicates the subject has renal decline or is at an increased risk of developing renal decline. In one embodiment, a decliner biomarker is a biomarker that is associated with increased risk of renal decline in a subject having T1D or T2D.

The term “end-stage renal disease (ESRD) marker”, “end-stage renal disease (ESRD) biomarker”, “end-stage kidney disease (ESKD)” or “end-stage kidney disease (ESKD) biomarker” refer to a molecule, e.g., a peptide, a protein (or polypeptide), which is associated with or is used to distinguish a disease activity, i.e., end-stage renal disease, in a subject from whom a sample or tissue is obtained.

The term “ESRD-associated protein” or “end stage renal disease-associated protein” refers to a protein (or polypeptide), or fragment thereof, which is associated with or is used to distinguish a disease activity or a condition, i.e., ESRD, in a subject from whom a sample or tissue is obtained. In one embodiment, a ESRD-associated protein(s) may include any one or a combination (or combinations) of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, and KIM1.

As used herein, the term “ESRD progressor”, “progressor” or “rapid progressor” refers to a subject with ESRD, or having an increased risk of developing ESRD. In one embodiment, a progressor has a level of at least one (or at least two, or at least three, etc.) biomarker, e.g., protein biomarker, that is statistically significantly higher than a non-progressor control level (or a normoalbuminuric control level, or a healthy control level, or a standard control level), and, as such, has (or has an increased risk of developing) ESRD. In certain embodiments, a non-progressor is a non-diabetic human subject.

As used herein, the term “estimated Glomerular Filtration Rate” or “eGFR,” refers to a means for estimating kidney function. In one embodiment, eGFR may be determined based on a measurement of serum creatinine levels (eGFR_{cr}). In one embodiment, eGFR may be determined based on a measurement of serum cystatin C levels (eGFR_{cys}).

As used herein, the term “expression” when used in connection with detecting the expression of a biomarker of the present disclosure, can refer to detecting transcription of the gene encoding a biomarker protein, to detecting translation of the biomarker protein, and/or detecting the biomarker protein which results from metabolism of a larger protein. To detect expression of a biomarker refers to the act of actively determining whether a biomarker is expressed or not. To quantitate expression refers to the act of determining the level of the given biomarker, e.g., ng/ml. Detecting and/or quantitating expression can include determining whether the biomarker expression is upregulated as compared to a known standard level, downregulated as compared to a known standard level, or substantially unchanged as compared to a known standard level. Therefore, the step of quantitating and/or detecting expression does not require that expression of the biomarker actually is upregulated or downregulated, but rather, can also include detecting no expression of the biomarker or detecting that the expression of the biomarker has not changed or is not different (i.e., detecting no significant expression of the biomarker or no significant change in expression of the biomarker as compared to a control).

As used herein, the term “known standard level”, “reference level” or “control level” refers to an accepted or pre-determined level of a biomarker which is used to compare the biomarker level derived from the sample of a subject. In one embodiment, when compared to the known standard level of a certain biomarker, deviation from the known standard level generally indicates either an improvement or deterioration in a disease state. In one embodiment, when compared to the known standard level of a certain biomarker, deviation from the known standard level generally indicates an increased or decreased likelihood of disease progression in a subject. Alternatively, when compared to the known standard level of a certain biomarker, equivalence to the known standard level generally indicates confirmation of the disease activity, confirmation of a non-disease state, or, if the biomarker level of the subject is obtained following therapeutic treatment for the disease, failure of a therapy to improve a patient's disease state. In one embodiment, the known standard level of a biomarker indicates an unaffected, i.e., non-disease, state of a subject who is not characterized as having renal decline or ESRD, or who is not at risk of developing renal decline or ESRD.

The term “level” or “amount” of a biomarker, as used herein, refers to the measurable quantity of a biomarker, e.g., a peptide, a protein (or polypeptide). The amount may be either (a) an absolute amount as measured in molecules, moles or weight per unit volume or cells or (b) a relative amount, e.g., measured by densitometric analysis.

The term “marker” or “biomarker,” as used herein, refers generally to a molecule, e.g., a peptide, a protein (or polypeptide), the expression of which in or on a sample derived from a mammalian tissue or cell can be detected, for example, by standard methods in the art (as well as methods disclosed herein), and is predictive or denotes a condition of the subject from which it was obtained. Where the biomarker is a protein, modulation or alteration of expression may encompass modulation through different post translational modifications. A biomarker (e.g. a peptide or a protein biomarker) may be used to distinguish disease activity, including improvements in a disease condition and/or deterioration of a disease condition, based on the level of the biomarker. Accordingly, in one embodiment, a biomarker useful in the present disclosure, is any molecule (or combination of molecules) the expression of which is regulated (up or down) in a subject with, or at risk of developing, a disease condition, e.g., renal decline (RD) and/or end-stage renal disease (ESRD), when compared to a normal control, i.e., an unaffected (healthy) subject. In one embodiment, selected sets of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty or more of the biomarkers as disclosed herein can be used as end-points for rapid diagnostics or prognostics for determining whether a patient has, or is at risk of developing a disease condition, e.g., renal decline (RD) or end-stage renal disease (ESRD).

As used herein, the term “non-decliner” refers to a subject who is not characterized as having renal decline (or who has a reduced risk of renal decline). A non-decliner may be characterized as a subject having certain levels of specific biomarkers as disclosed herein. In one embodiment, a non-decliner is a subject having T1D or T2D, but who has a lower risk of developing renal decline based on lower or comparable levels of at least one biomarker (e.g., in comparison to a normoalbuminuric control level, or a healthy control level, or a standard control level, or a non-T1D control, or a non-T2D control). In one embodiment, a non-decliner is defined as a subject who has a level of at least one biomarker that is statistically significantly lower than a decliner control level or is comparable to a normoalbuminuric control level, or a healthy control level, or a standard control level, or a non-T1D control level, or a non-T2D control level.

As used herein, the term “non-progressor” refers to a subject who is not characterized as having ESRD (or who has a reduced risk of developing ESRD). A non-progressor may be characterized as a subject having certain levels of specific biomarkers as disclosed herein. In one embodiment, a non-progressor is a subject having T1D or T2D, but who has a lower risk of progressing to ESRD based on lower or comparable levels of at least one biomarker (e.g., in comparison to a normoalbuminuric control level, or a healthy control level, or a standard control level, a non-T1D control, or a non-T2D control). In one embodiment, a non-progressor is defined as a subject who has a level of at least one biomarker that is statistically significantly lower than a progressor control level or is comparable to a normoalbuminuric control level, or a healthy control level, or a standard control level, or a non-T1D control level, or a non-T2D control level.

As used herein, the term “progressor biomarker” refers to a biomarker that is associated ESRD (or with increased risk of developing ESRD) in a subject. In one embodiment, an increase in the level of a progressor biomarker in a subject relative to a non-progressor level (or a normoalbuminuric control level, or a healthy control level, or a standard control level) indicates the subject has ESRD or is at an increased risk of developing ESRD. In one embodiment, a progressor biomarker is a biomarker that is associated with increased risk of developing ESRD in a subject having T1D or T2D.

The term “RD-associated protein” or “renal decline-associated protein” refers to a peptide or a protein (or polypeptide), or fragment thereof, which is associated with or is used to distinguish a disease activity or a condition, i.e., renal decline, in a subject from whom a sample or tissue is obtained. In one embodiment, a RD-associated protein(s) may include any one or a combination (or combinations) of TNF-R1, TNF-R2, CD27, LTBR, TNF-RSF6B, TNF-RSF10A, TNF-RSF4, EDA2R, RELT, CD160, IL-1RT1, DLL1, LAYN, MMP7, NBL1, PI3, WFDC2, EFNA4, EPHA2, GFR-alpha-1, and KIM1.

As used herein, the term “renal decline” or “RD,” refers to a condition associated with impaired renal function and is defined as an estimated Glomerular Filtration Rate (eGFR) change of at least <−3 ml/min/year. In one embodiment, an eGFR change of between −5 ml/min/year and −3 ml/min/year indicates slow or moderate renal decline. In another embodiment, an eGFR change of between −80 ml/min/year and −5 ml/min/year indicates fast renal decline.

As used herein, the term “estimated Glomerular Filtration Rate” or “eGFR,” refers to a means for estimating kidney function. In one embodiment, eGFR may be determined based on a measurement of serum creatinine levels. In another embodiment, eGFR may be determined based on a measurement of serum cystatin C levels. eGFR can be determined using the CKD-EPI creatinine equation, as described, for example, in Levey et al. (Ann Intern Med 150(9): 604-61221 (2009)).

The term “renal decline marker” or “renal decline biomarker” refers to a molecule, e.g., a peptide, a protein (or polypeptide), which is associated with or is used to distinguish a disease activity, i.e., renal decline, in a subject from whom a sample or tissue is obtained.

As used herein, the term “renal decliner”, “decliner” or “rapid decliner” refers to a subject with renal decline, or having an increased risk for renal decline. In one embodiment, a decliner has a level of at least one (or at least two, etc.) biomarker that is statistically significantly higher than a non-decliner control level (or a normoalbuminuric control level, or a healthy control level, or a standard control level), and, as such, has (or an increased risk of developing) renal decline.

The term “sample” or “biological sample” as used herein refers to cells or tissue obtained from a subject. The source of the tissue or cell sample may be solid tissue (as from a fresh, frozen and/or preserved organ or tissue sample or biopsy or aspirate); whole blood or any blood constituents; or bodily fluids, such as serum, plasma, urine, saliva, sweat or synovial fluid. In one embodiment, the sample is a plasma sample obtained from a human subject. In another embodiment, the sample is a urine sample obtained from a human subject.

The term “subject” or “patient,” as used interchangeably herein, refers to either a human or non-human animal. In one embodiment, a subject is a human subject.

The term “therapeutically effective amount” refers to an amount which, when administered to a subject, achieves a desired effect on the subject. The exact amount will depend on the purpose of the treatment, and may be ascertainable by one skilled in the art using known techniques. As is known in the art, adjustments for systemic versus localized delivery, age, body weight, general health, sex, diet, time of administration, drug interaction and the severity of the condition may be necessary, and may be ascertainable with routine experimentation by those skilled in the art. For example, an effective amount of an agent described herein for administration to the living subject is an amount that prevents and/or treats RD or ESRD. For example, for an antagonist of any one of the biomarkers as disclosed herein, a therapeutically effective amount can be an amount that has been shown to provide an observable therapeutic benefit compared to baseline clinically observable signs and symptoms of renal decline and/or ESRD.

### II. Biomarkers for Determining Risk of RD and ESRD and Uses Thereof

The present disclosure is based, at least in part, on the discovery that certain biomarkers are associated with renal decline and/or progression to ESRD, for example, in subjects with T1D or T2D. In particular, the markers described herein are protein markers. As described in the Examples below, the disclosure provides biomarkers whose levels can be used to predict whether a subject has (or is at risk of developing) renal decline and/or has (or is at risk of developing) end stage renal disease. As described herein, biomarkers that were highly statistically associated with fast renal decline (an eGFR slope of between −80 ml/min/year and −5 ml/min/year) and high risk of progression to ESRD were identified in diabetic patients. These biomarkers were identified as predictors of whether a subject has (or is at risk of developing renal decline and/or has (or is at risk of developing) end stage renal disease (ESRD or ESKD).

Thus, in one embodiment, the disclosure relates to biomarkers (e.g., circulating plasma protein(s)) highly statistically associated with fast renal decline (an eGFR slope of between −80 ml/min/year and −5 ml/min/year) and/or high risk of progression to ESRD. In some embodiments, these biomarkers may be used to determine whether a subject, e.g., a T1D or T2D subject, has renal decline or has an increased risk for developing renal decline. In other embodiments, these biomarkers may also be used to determine whether a subject, e.g., a T1D or T2D subject, has ESRD or has an increased risk for developing ESRD.

The protein markers that can be used in the compositions and methods disclosed herein include the following, alone or in combination:

TNF-R1

TNF-R1 (also known as Tumor Necrosis Factor Receptor Superfamily Member 1A, Tumor Necrosis Factor Receptor 1A Isoform Beta, Tumor Necrosis Factor-Alpha Receptor, Tumor Necrosis Factor Binding Protein 1, Tumor Necrosis Factor Receptor Type 1, TNF-RI, TNFR1, TNF-R, CD120a Antigen, CD120a, P55, TNF-R55, P55-R, P60, TNFR60, TBP1, FPF). Findings suggest an important role of phosphorylation of the TNF-R1 by number of protein kinases, such as protein kinase C-δ, PI3K and TNF-R1 associated kinase (Ihnatko et al., Gen. Physiol. Biophy., 26, 159-167; 2007). The amino acid sequence of human TNF-R1 is as follows (UniProt Accession No. P19438 canonical sequence P19438-1):

(SEQ ID NO: 1)
MGLSTVPDLLLPLVLLELLVGIYPSGVIGLVPHLGDREKRDSVCP
QGKYIHPQNNSICCTKCHKGTYLYNDCPGPGQDTDCRECESGSFT
ASENHLRHCLSCSKCRKEMGQVEISSCTVDRDTVCGCRKNQYRHY
WSENLFQCFNCSLCLNGTVHLSCQEKQNTVCTCHAGFFLRENECV
SCSNCKKSLECTKLCLPQIENVKGTEDSGTTVLLPLVIFFGLCLL
SLLFIGLMYRYQRWKSKLYSIVCGKSTPEKEGELEGTTTKPLAPN
PSFSPTPGFTPTLGFSPVPSSTFTSSSTYTPGDCPNFAAPRREVA
PPYQGADPILATALASDPIPNPLQKWEDSAHKPQSLDTDDPATLY
AVVENVPPLRWKEFVRRLGLSDHEIDRLELONGRCLREAQYSMLA
TWRRRTPRREATLELLGRVLRDMDLLGCLEDIEEALCGPAALPPA
PSLLR

TNF-R2

TNF-R2 (also known as Tumor Necrosis Factor Receptor Superfamily Member 1B, Tumor Necrosis Factor Receptor Type II, Tumor Necrosis Factor Receptor 2, Tumor Necrosis Factor Binding Protein 2, Tumor Necrosis Factor Beta Receptor, P80 TNF-Alpha Receptor, TNF-RII, TNFR2, TNFR2, TNFBR, TBPII, CD120b Antigen, CD120b, P75, P75 TNF Receptor, TNF-R75, P75TNFR, TNFR80) is a member of the TNF-receptor superfamily. TNF-R2 is highly expressed and typically found in cells of the immune system. TNF-R2 phosphorylation plays a role in TNF-R2 signaling (Ihnatko et al., Gen. Physiol. Biophy., 26, 159-167; 2007). The human TNF-R2 amino acid sequence is as follows (UniProt Accession No. P20333 canonical sequence P20333-1):

(SEQ ID NO: 2)
MAPVAVWAALAVGLELWAAAHALPAQVAFTPYAPEPGSTCRLREY
YDQTAQMCCSKCSPGQHAKVFCTKTSDTVCDSCEDSTYTQLWNWV
PECLSCGSRCSSDQVETQACTREQNRICTCRPGWYCALSKQEGCR
LCAPLRKCRPGFGVARPGTETSDVVCKPCAPGTFSNTTSSTDICR
PHQICNVVAIPGNASMDAVCTSTSPTRSMAPGAVHLPQPVSTRSQ
HTQPTPEPSTAPSTSFLLPMGPSPPAEGSTGDFALPVGLIVGVTA
LGLLIIGVVNCVIMTQVKKKPLCLQREAKVPHLPADKARGTQGPE
QQHLLITAPSSSSSSLESSASALDRRAPTRNQPQAPGVEASGAGE
ARASTGSSDSSPGGHGTQVNVTCIVNVCSSSDHSSQCSSQASSTM
GDTDSSPSESPKDEQVPFSKEECAFRSQLETPETLLGSTEEKPLP

CD27

CD27 (also known as Tumor Necrosis Factor Receptor Superfamily Member 7, T-Cell Activation Antigen CD27, CD27L Receptor, CD27 Antigen, CD27 Molecule, TNFRSF7, TNF-RSF7, T14, T Cell Activation Antigen S152, LPFS2, S152, Tp55) is expressed on naïve CD4+ and CD8+ T cells. The cytoplasmic tail of CD27 contains motifs that bind TNFR-associated factors (TRAFs), and the subsequent ubiquitination of TRAFs activates both canonical and noncanonical nuclear factor kappa B (NF-KB) pathways as well as the c-Jun-N-terminal kinase (JNK)-signaling cascade. The amino acid sequence of CD27 can be found at, for example, UniProt Accession No. P26842. The human CD27 amino acid sequence is as follows:

(SEQ ID NO: 3)
MARPHPWWLCVLGTLVGLSATPAPKSCPERHYWAQGKLCCQMCEP
GTFLVKDCDQHRKAAQCDPCIPGVSFSPDHHTRPHCESCRHCNSG
LLVRNCTITANAECACRNGWQCRDKECTECDPLPNPSLTARSSQA
LSPHPQPTHLPYVSEMLEARTAGHMQTLADFRQLPARTLSTHWPP
QRSLCSSDFIRILVIFSGMFLVFTLAGALFLHQRRKYRSNKGESP
VEPAEPCHYSCPREEEGSTIPIQEDYRKPEPACSP

LTBR

LTBR (also known as Lymphotoxin Beta Receptor, Tumor Necrosis Factor Receptor Superfamily Member 3, Tumor Necrosis Factor Receptor 2-Related Protein, Tumor Necrosis Factor Receptor Type III, Tumor Necrosis Factor C Receptor, D125370, TNFRSF3, TNFCR, TNFR3, TNF-R3, Lymphotoxin B Receptor, LT-BETA-R, TNF-R-III, TNFR2-RP, TNF-RIII, TNFR-III, TNFR-RP) is a receptor for the heterotrimeric lymphotoxin containing LTA and LTB, and for TNFS14/LIGHT. LTBR promotes apoptosis via TRAF3 and TRAFS, and may play a role in the development of lymphoid organs (Bista et al., Signal Transduction; Volume 285, issue 17, P12971-12978, 2010). The amino acid sequence of human LTBR is as follows (UniProt Accession No. P36941 canonical sequence P36941-1):

(SEQ ID NO 4)
MLLPWATSAPGLAWGPLVLGLFGLLAASQPQAVPPYASENQTCRD
QEKEYYEPQHRICCSRCPPGTYVSAKCSRIRDTVCATCAENSYNE
HWNYLTICQLCRPCDPVMGLEEIAPCTSKRKTQCRCQPGMFCAAW
ALECTHCELLSDCPPGTEAELKDEVGKGNNHCVPCKAGHFQNTSS
PSARCQPHTRCENQGLVEAAPGTAQSDTTCKNPLEPLPPEMSGTM
LMLAVLLPLAFFLLLATVFSCIWKSHPSLCRKLGSLLKRRPQGEG
PNPVAGSWEPPKAHPYFPDLVQPLLPISGDVSPVSTGLPAAPVLE
AGVPQQQSPLDLTREPQLEPGEQSQVAHGTNGIHVTGGSMTITGN
IYIYNGPVLGGPPGPGDLPATPEPPYPIPEEGDPGPPGLSTPHQE
DGKAWHLAETEHCGATPSNRGPRNQFITHD

TNF-RSF6B

TNF-RSF6B (also known as TNF Receptor Superfamily Member 6b, Tumor Necrosis Factor Receptor Superfamily, Member 6b, Decoy, Tumor Necrosis Factor Receptor Superfamily Member 6B, TNFRSF6B, Decoy Receptor For Fas Ligand, DCR3, M68, TR6, Decoy Receptor 3 Variant 1, Decoy Receptor 3 Variant 2, Decoy Receptor 3, DJ583P15.1.1, M68E, DcR3) belongs to the tumor necrosis factor receptor family, but lacks transmembrane and cytoplasmic domains in its sequence. Serum TNFRSF6B level is markedly elevated in patients with chronic kidney disease (Tseng et al., Modern Pathology volume 26, pages 984-994, 2013; Chen et al., J Immunol Methods; 285:63-70, 2004). The amino acid sequence of human TNF-RSF6B is as follows:

(SEQ ID NO: 5)
MRALEGPGLSLLCLVLALPALLPVPAVRGVAETPTYPWRDAETGE
RLVCAQCPPGTFVQRPCRRDSPTTCGPCPPRHYTQFWNYLERCRY
CNVLCGEREEEARACHATHNRACRCRTGFFAHAGFCLEHASCPPG
AGVIAPGTPSQNTQCQPCPPGTFSASSSSSEQCQPHRNCTALGLA
LNVPGSSSHDTLCTSCTGFPLSTRVPGAEECERAVIDFVAFQDIS
IKRLQRLLQALEAPEGWGPTPRAGRAALQLKLRRRLTELLGAQDG
ALLVRLLQALRVARMPGLERSVRERFLPVH

FR-Alpha

FR-alpha (also known as Folate Receptor Alpha, Folate Receptor 1, Ovarian Tumor-Associated Antigen MOv18, Adult Folate-Binding Protein, Folate Receptor 1 (Adult), Folate Receptor, Adult, KB Cells FBP, FOLR, Folate Binding Protein, FBP, FRalpha, FRA, FOLR1) is a 38-40 Da glycosylphosphatidylinositol (GPI)-anchored protein that binds plasma folate (5-methyltetrahydrofolate) with high affinity (KD ˜1 nM) and transports it into the cell via endocytosis. FR-alpha has also been shown to be expressed on a number of epithelial tumors including ovarian, endometrial, lung adenocarcinoma, renal clear cell cancer, and triple negative breast cancer (Somers et al., Biomarker Insights: 9 29-37, 2014). The amino acid sequence of human FR-alpha is as follows (UniProt Accession No. P15328 canonical sequence P15328-1):

(SEQ ID NO: 6)
MAQRMTTQLLLLLVWVAVVGEAQTRIAWARTELLNVCMNAKHHKE
KPGPEDKLHEQCRPWRKNACCSTNTSQEAHKDVSYLYRFNWNHCG
EMAPACKRHFIQDTCLYECSPNLGPWIQQVDQSWRKERVLNVPLC
KEDCEQWWEDCRTSYTCKSNWHKGWNWTSGFNKCAVGAACQPFHF
YFPTPTVLCNEIWTHSYKVSNYSRGSGRCIQMWFDPAQGNPNEEV
ARFYAAAMSGAGPWAAWPFLLSLALMLLWLLS

INF-RSF10A

TNF-RSF10A (also known as TNFRSF10A TNF Receptor Superfamily Member 10a, Tumor Necrosis Factor Receptor Superfamily, Member 10a, TNF-Related Apoptosis-Inducing Ligand Receptor 1, Death Receptor 4, TRAIL Receptor 1, TRAIL-R1, TRAILR1, APO2, DR4, Tumor Necrosis Factor Receptor Superfamily Member 10a Variant 2, Cytotoxic TRAIL Receptor, CD261 Antigen, TRAILR-1, CD261) is a cell surface receptor that bind to TRAIL 3 and mediate the extrinsic pathway of apoptosis. The up-regulation of TNFRSF10A expression usually occurs at the transcriptional level via transcriptional factors AP-1, TP53, or NF-κB (Li et al., Gene Regulation, volume 290, issue 17, P11108-11118, 2015). The amino acid sequence of human RSF10A is as follows (UniProt Accession No. 000220):

(SEQ ID NO: 7)
MAPPPARVHLGAFLAVTPNPGSAASGTEAAAATPSKVWGSSAGRI
EPRGGGRGALPTSMGQHGPSARARAGRAPGPRPAREASPRLRVHK
TFKFVVVGVLLQVVPSSAATIKLHDQSIGTQQWEHSPLGELCPPG
SHRSEHPGACNRCTEGVGYTNASNNLFACLPCTACKSDEEERSPC
TTTRNTACQCKPGTFRNDNSAEMCRKCSRGCPRGMVKVKDCTPWS
DIECVHKESGNGHNIWVILVVTLVVPLLLVAVLIVCCCIGSGCGG
DPKCMDRVCFWRLGLLRGPGAEDNAHNEILSNADSLSTFVSEQQM
ESQEPADLTGVTVQSPGEAQCLLGPAEAEGSQRRRLLVPANGADP
TETLMLFFDKFANIVPFDSWDQLMRQLDLTKNEIDVVRAGTAGPG
DALYAMLMKWVNKTGRNASIHTLLDALERMEERHAREKIQDLLVD
SGKFIYLEDGTGSAVSLE

TNF-RSF4

TNF-RSF4 (also known as TNF Receptor Superfamily Member 4, TAX Transcriptionally-Activated Glycoprotein 1 Receptor, Tumor Necrosis Factor Receptor Superfamily Member 4, OX40L Receptor, ACT35 Antigen, CD134 Antigen, TXGP1L, Tax-Transcriptionally Activated Glycoprotein 1 Receptor, Tumor Necrosis Factor Receptor Superfamily, Member 4, Lymphoid Activation Antigene ACT35, OX40 Cell Surface Antigen, OX40 Homologue, ATC35 Antigen, OX40 Antigen, ACT35, CD134, IMD16, OX40) is on chromosome 4 and is proposed to signal upon ligation to its homotrimerized ligand OX40L. TNF-RSF4 is reported to have highly similar activity in T cell activation despite its unique cytoplasmic domain structure and signaling pathway (Schreiber et al., J Immunol, 189:3311-3318, 2012). The amino acid sequence of human TNF-RSF4 is as follows (UniProt Accession No. P43489):

(SEQ ID NO: 8)
MCVGARRLGRGPCAALLLLGLGLSTVTGLHCVGDTYPSNDRCCHE
CRPGNGMVSRCSRSQNTVCRPCGPGFYNDVVSSKPCKPCTWCNLR
SGSERKQLCTATQDTVCRCRAGTQPLDSYKPGVDCAPCPPGHFSP
GDNQACKPWTNCTLAGKHTLQPASNSSDAICEDRDPPATQPQETQ
GPPARPITVQPTEAWPRTSQGPSTRPVEVPGGRAVAAILGLGLVL
GLLGPLAILLALYLLRRDQRLPPDAHKPPGGGSFRTPIQEEQADA
HSTLAKI

TNF-RSF14

TNF-RSF14 (also known as TNF Receptor Superfamily Member 14, Tumor Necrosis Factor Receptor Superfamily, Member 14 (Herpesvirus Entry Mediator), Tumor Necrosis Factor Receptor Superfamily Member 14, Herpes Virus Entry Mediator A, Tumor Necrosis Factor Receptor-Like Gene2, Tumor Necrosis Factor Receptor-Like 2, TNFRSF14, Herpesvirus Entry Mediator, Herpesvirus Entry Mediator A, HVEA, HveA, HVEM, TR2, CD40-Like Protein, CD270 Antigen, CD270, LIGHTR, ATAR) is located on the short arm of chromosome 1p36 and encodes a type I trans membrane molecule that serves as a molecular switch by interacting with different ligands to regulate a series of immune responses. TNF-RSF14 deficiency promotes development of follicular lymphoma in vivo and establishes a favorable immune environment. The amino acid sequence of human TNF-RSF14 is as follows (UniProt Accession No. Q92956 canonical sequence Q92956-1):

(SEQ ID NO: 9)
MEPPGDWGPPPWRSTPKTDVLRLVLYLTFLGAPCYAPALPSCKED
EYPVGSECCPKCSPGYRVKEACGELTGTVCEPCPPGTYIAHLNGL
SKCLQCQMCDPAMGLRASRNCSRTENAVCGCSPGHFCIVQDGDHC
AACRAYATSSPGQRVQKGGTESQDTLCQNCPPGTFSPNGTLEECQ
HQTKCSWLVTKAGAGTSSSHWVWWFLSGSLVIVIVCSTVGLIICV
KRRKPRGDVVKVIVSVQRKRQEAEGEATVIEALQAPPDVTTVAVE
ETIPSFTGRSPNH

EDA2R

EDA2R (also known as Ectodysplasin A2 Receptor, Tumor Necrosis Factor Receptor Superfamily Member 27, X-Linked Ectodysplasin-A2 Receptor, EDA-A2 Receptor, TNFRSF27, XEDAR, Tumor Necrosis Factor Receptor Superfamily Member XEDAR, Ectodysplasin A2 Isoform Receptor, EDA-A2R, EDAA2R) encodes a transmembranal receptor that belongs to the tumor necrosis factor (TNF)-receptor superfamily EDA2R, as well as its paralog, EDAR, bind the ectodysplasin ligands EDAA2 and EDA-A1, respectively; which are two alternatively spliced forms of the EDA gene. EDA2R is also identified as a p53 target gene that mediates anoikis, suggesting EDA2R is a tumor suppressor that limits colorectal cancer progression (Brosh et al., FEBS Letters 584, 2473-2477, 2010). The amino acid sequence of human EDA2R is as follows (UniProt Accession No. Q9HAV5 canonical sequence Q9HAV5-1):

(SEQ ID NO: 10)
MDCQENEYWDQWGRCVTCQRCGPGQELSKDCGYGEGGDAYCTACP
PRRYKSSWGHHRCQSCITCAVINRVQKVNCTATSNAVCGDCLPRF
YRKTRIGGLQDQECIPCTKQTPTSEVQCAFQLSLVEADTPTVPPQ
EATLVALVSSLLVVFTLAFLGLFFLYCKQFFNRHCQRGGLLQFEA
DKTAKEESLFPVPPSKETSAESQVSENIFQTQPLNPILEDDCSST
SGFPTQESFTMASCTSESHSHWVHSPIECTELDLQKFSSSASYTG
AETLGGNTVESTGDRLELNVPFEVPSP

RELT

RELT (also known as RELT TNF Receptor, Receptor Expressed In Lymphoid Tissues, Tumor Necrosis Factor Receptor Superfamily Member 19L, TNF-RSF19L, RELT Tumor Necrosis Factor Receptor, TNFRSF19L, Tumor Necrosis Factor Receptor Superfamily, Member 19-Like, AI3C, TRLT) is a member of the tumor necrosis factor receptor superfamily (TNFRSF). Mutations in RELT cause autosomal recessive amelogenesis imperfect (Kim et al., Clin. Genet. 95, 375-383, 2019). The amino acid sequence of human RELT is as follows (UniProt Accession No. Q969Z4):

(SEQ ID NO: 11)
MKPSLLCRPLSCFLMLLPWPLATLTSTTLWQCPPGEEPDLDPGQG
TLCRPCPPGTFSAAWGSSPCQPHARCSLWRRLEAQVGMATRDTLC
GDCWPGWFGPWGVPRVPCQPCSWAPLGTHGCDEWGRRARRGVEVA
AGASSGGETRQPGNGTRAGGPEETAAQYAVIAIVPVFCLMGLLGI
LVCNLLKRKGYHCTAHKEVGPGPGGGGSGINPAYRTEDANEDTIG
VLVRLITEKKENAAALEELLKEYHSKQLVQTSHRPVSKLPPAPPN
VPHICPHRHHLHTVQGLASLSGPCCSRCSQKKWPEVLLSPEAVAA
TTPVPSLLPNPTRVPKAGAKAGRQGEITILSVGRFRVARIPEQRT
SSMVSEVKTITEAGPSWGDLPDSPQPGLPPEQQALLGSGGSRTKW
LKPPAENKAEENRYVVRLSESNLVI

CD160

CD160 (also known as CD160 Molecule, CD160 Antigen, Natural Killer Cell Receptor BY55, BY55, Natural Killer Cell Receptor, Immunoglobulin Superfamily Member, CD160 Transmembrane Isoform, CD160-Delta Ig, NK28, NK1) is a cell-surface antigen expressed on natural killer (NK) cells, CD8+ cells, a small subset of CD4+ cells, and all intraepithelial lymphocytes (IELs). A soluble form of CD160, shed from NK cells, has also been reported to inhibit cell-mediated cytotoxicity (Liu et al., Structure, Volume 27, Issue 8, Pages 1286-1295.e4, 2019). Human CD160 also binds to herpesvirus entry mediator (HVEM), a TNF family member, with much higher affinity than to MHC class I, and leads to suppressed T cell responses in vitro (Tu et al., J Exp Med, 212 (3): 415-429, 2015). The amino acid sequence of human CD160 is as follows (UniProt Accession No. 095971):

(SEQ ID NO: 12)
MLLEPGRGCCALAILLAIVDIQSGGCINITSSASQEGTRLNLICT
VWHKKEEAEGFVVFLCKDRSGDCSPETSLKQLRLKRDPGIDGVGE
ISSQLMFTISQVTPLHSGTYQCCARSQKSGIRLQGHFFSILFTET
GNYTVTGLKQRQHLEFSHNEGTLSSGFLQEKVWVMLVTSLVALQA
L

IL-1RT1

IL-1RT1 (also known as Interleukin 1 Receptor Type 1, CD121 Antigen-Like Family Member A, Interleukin-1 Receptor Type 1, Interleukin-1 Receptor Type I, Interleukin-1 Receptor Alpha, IL-1R-Alpha, IL-1RT-1, IL-1R-1, IL1RA, IL1R, P80, Interleukin 1 Receptor Alpha, Type I, Interleukin 1 Receptor, Type I, CD121a Antigen, D2S1473, CD121A, IL1RT1) is the receptor of IL-1α and IL-1β. The binding of IL-1α or IL-1β triggers the dimerization with IL-1R receptor accessory protein (IL-1RAP), resulting in activation of NF-κB (Buzzelli et al., Oncotarget, 6(2): 679-695, 2015). The amino acid sequence of human IL-1RT1 is as follows (UnitProt Accession No. P14778):

(SEQ ID NO: 13)
MKVLLRLICFIALLISSLEADKCKEREEKIILVSSANEIDVRPCP
LNPNEHKGTITWYKDDSKTPVSTEQASRIHQHKEKLWFVPAKVED
SGHYYCVVRNSSYCLRIKISAKFVENEPNLCYNAQAIFKQKLPVA
GDGGLVCPYMEFFKNENNELPKLQWYKDCKPLLLDNIHFSGVKDR
LIVMNVAEKHRGNYTCHASYTYLGKQYPITRVIEFITLEENKPTR
PVIVSPANETMEVDLGSQIQLICNVTGQLSDIAYWKWNGSVIDED
DPVLGEDYYSVENPANKRRSTLITVLNISEIESRFYKHPFTCFAK
NTHGIDAAYIQLIYPVTNFQKHMIGICVTLTVIIVCSVFIYKIFK
IDIVLWYRDSCYDFLPIKASDGKTYDAYILYPKTVGEGSTSDCDI
FVFKVLPEVLEKQCGYKLFIYGRDDYVGEDIVEVINENVKKSRRL
IIILVRETSGFSWLGGSSEEQIAMYNALVQDGIKVVLLELEKIQD
YEKMPESIKFIKQKHGAIRWSGDFTQGPQSAKTRFWKNVRYHMPV
QRRSPSSKHQLLSPATKEKLQREAHVPLG

DLL1

DLL1 (also known as Delta Like Canonical Notch Ligand 1, Drosophila Delta Homolog 1, Delta-Like Protein 1, H-Delta-1, Epididymis Secretory Sperm Binding Protein, Delta (Drosophila)-Like 1, Delta-Like 1 (Drosophila), DELTA1, Deltal, Delta, DL1) belongs to the Delta/Jagged family of transmembrane proteins. DLL1 is implicated in regulating cell fate determination, proliferation, stem cell self-renewal and apoptosis. DLL1 may also play a role in cell-to-cell communication (Hildebrand et al., Front Cell Infect Microbiol.; 9: 267, 2019). The amino acid sequence of human DLL1 as follows (UniProt Accession No. 000548 canonical sequence 000548-1):

(SEQ ID NO: 14)
MGSRCALALAVLSALLCQVWSSGVFELKLQEFVNKKGLLGNRNCC
RGGAGPPPCACRTFFRVCLKHYQASVSPEPPCTYGSAVTPVLGVD
SFSLPDGGGADSAFSNPIRFPFGFTWPGTFSLIIEALHTDSPDDL
ATENPERLISRLATQRHLTVGEEWSQDLHSSGRTDLKYSYRFVCD
EHYYGEGCSVFCRPRDDAFGHFTCGERGEKVCNPGWKGPYCTEPI
CLPGCDEQHGFCDKPGECKCRVGWQGRYCDECIRYPGCLHGTCQQ
PWQCNCQEGWGGLFCNQDLNYCTHHKPCKNGATCTNTGQGSYTCS
CRPGYTGATCELGIDECDPSPCKNGGSCTDLENSYSCTCPPGFYG
KICELSAMTCADGPCFNGGRCSDSPDGGYSCRCPVGYSGFNCEKK
IDYCSSSPCSNGAKCVDLGDAYLCRCQAGFSGRHCDDNVDDCASS
PCANGGTCRDGVNDFSCTCPPGYTGRNCSAPVSRCEHAPCHNGAT
CHERGHRYVCECARGYGGPNCQFLLPELPPGPAVVDLTEKLEGQG
GPFPWVAVCAGVILVLMLLLGCAAVVVCVRLRLQKHRPPADPCRG
ETETMNNLANCQREKDISVSIIGATQIKNTNKKADFHGDHSADKN
GFKARYPAVDYNLVQDLKGDDTAVRDAHSKRDTKCQPQGSSGEEK
GTPTTLRGGEASERKRPDSGCSTSKDTKYQSVYVISEEKDECVIA
TEV

LAYN

LAYN (also known as Layilin) is a C-type lectin domain-containing membrane glycoprotein having about 55 kDa. As a type I transmembrane protein, LAYN has the potential to mediate signals from extracellular matrix (ECM) to the cell cytoskeleton, and, based on LAYN's homology to E-selectin's ligand-binding region (Bono et al., Molecular Biology of the Cell, Vol. 12, No. 4, 2017). LAYN is selectively expressed on highly activated, clonally expanded, but phenotypically exhausted CD8+ T cells in human melanoma. Lineage-specific deletion of layilin on murine CD8+ T cells reduced their accumulation in tumors and increased tumor growth in vivo (Mahuron et al., J Exp Med 217 (9): e20192080, 2020). The amino acid sequence of human LAYN is as follows (UniProt Accession No. Q6UX15 canonical sequence Q6UX15-1):

(SEQ ID NO: 15)
MRPGTALQAVLLAVLLVGLRAATGRLLSASDLDLRGGQPVCRGGT
QRPCYKVIYFHDTSRRLNFEEAKEACRRDGGQLVSIESEDEQKLI
EKFIENLLPSDGDFWIGLRRREEKQSNSTACQDLYAWTDGSISQF
RNWYVDEPSCGSEVCVVMYHQPSAPAGIGGPYMFQWNDDRCNMKN
NFICKYSDEKPAVPSREAEGEETELTTPVLPEETQEEDAKKTFKE
SREAALNLAYILIPSIPLLLLLVVTTVVCWVWICRKRKREQPDPS
TKKQHTIWPSPHQGNSPDLEVYNVIRKQSEADLAETRPDLKNISF
RVCSGEATPDDMSCDYDNMAVNPSESGFVTLVSVESGFVTNDIYE
FSPDQMGRSKESGWVENEIYGY

MMP7

MMP7 (also known as Matrix Metallopeptidase 7, Matrilysin, Matrix Metalloproteinase 7 (Matrilysin, Uterine), Matrix Metalloproteinase-7, Uterine Metalloproteinase, Pump-1 Protease, Matrin, MPSL1, MMP-7, Matrix Metallopeptidase 7 (Matrilysin, Uterine), Uterine Matrilysin, EC 3.4.24.23, EC 3.4.24, PUMP-1, PUMP1) is a member of a family of zinc-dependent endopeptidases. MMP7 is expressed predominantly in epithelial cells of glandular tissue, unlike other MMPs, which are often stromally derived. MMP7 is overexpressed in PanINs and PDA and correlates with decreased survival and possibly tumor size, lymph node involvement, and distant metastasis. It is also suggested that MMP7 is involved in disease progression by dictating the invasive and metastatic capacity of PDA cells (Fukuda et al., Cancer Cell, 19, 441-455, 2011). The amino acid sequence of human MMP7 is as follows (UniProt Accession No. P09237):

(SEQ ID NO: 16)
MRLTVLCAVCLLPGSLALPLPQEAGGMSELQWEQAQDYLKRFYLY
DSETKNANSLEAKLKEMQKFFGLPITGMLNSRVIEIMQKPRCGVP
DVAEYSLFPNSPKWTSKVVTYRIVSYTRDLPHITVDRLVSKALNM
WGKEIPLHFRKVVWGTADIMIGFARGAHGDSYPFDGPGNTLAHAF
APGTGLGGDAHFDEDERWTDGSSLGINFLYAATHELGHSLGMGHS
SDPNAVMYPTYGNGDPQNFKLSQDDIKGIQKLYGKRSNSRKK

NBL1

NBL1 (also known as NBL1, DAN Family BMP Antagonist, Neuroblastoma Suppressor Of Tumorigenicity 1, Neuroblastoma Candidate Region, Suppression Of Tumorigenicity 1, Differential Screening-Selected Gene Aberrant In Neuroblastoma, Neuroblastoma 1, DAN Family BMP Antagonist, DAN Domain Family Member 1, DAND1, DAN, Neuroblastoma, Suppression Of Tumorigenicity 1, Zinc Finger Protein DAN, Protein N03, D1S1733E, NO3, NB) is a member of a BMP antagonist family NBL1 was originally proposed to be a tumor suppressor gene based on its transcriptional pattern, whereby it was significantly down-regulated in v-src-transformed rat fibroblasts. NBL1 acts as a potential BMP antagonist. NBL1 is able to interact with BMP2 in vitro, and GDF5. NBL1 also acts as a modulator that balances GDF9 and BMP signaling during ovarian folliculogenesis (Hung et al., Biology of Reproduction, Volume 86, Issue 5, 158, 1-9, 2012). The amino acid sequence of human NBL1 is as follows (UniProt Accession No. P41271 canonical sequence P41271-1):

(SEQ ID NO: 17)
MMLRVLVGAVLPAMLLAAPPPINKLALFPDKSAWCEAKNITQIVG
HSGCEAKSIQNRACLGQCFSYSVPNTFPQSTESLVHCDSCMPAQS
MWEIVTLECPGHEEVPRVDKLVEKILHCSCQACGKEPSHEGLSVY
VQGEDGPGSQPGTHPHPHPHPHPGGQTPEPEDPPGAPHTEEEGAE
D

WFDC2

WFDC2 (also known as WAP Four-Disulfide Core Domain 2, WAP Four-Disulfide Core Domain Protein 2, Major Epididymis-Specific Protein E4, Putative Protease Inhibitor WAPS, Epididymal Secretory Protein E4, Epididymal Protein 4, WAPS, HE4, Epididymis-Specific, Whey-Acidic Protein Type, Four-Disulfide Core, Epididymis Secretory Sperm Binding Protein, WAP Domain Containing Protein HE4-V4, DJ461P17.6, EDDM4) encodes the small secretory protein human epididymis protein 4 (HE4), is widely upregulated in ovarian cancer. WFDC2 has been identified as a tumor suppressor which inhibits the metastasis of prostate cancer in vitro and in vivo, and that WFDC2 binds to the extracellular domain of epidermal growth factor receptor (EGFR) (Xiong et al., Cell Death & Disease, volume 11, 537, 2020). The amino acid sequence of human WFDC2 is as follows (UniProt Accession No. Q14508 canonical sequence Q14508-1):

(SEQ ID NO: 18)
MPACRLGPLAAALLLSLLLFGFTLVSGTGAEKTGVCPELQADQNC
TQECVSDSECADNLKCCSAGCATFCSLPNDKEGSCPQVNINFPQL
GLCRDQCQVDSQCPGQMKCCRNGCGKVSCVTPNF

EFNA4

EFNA4 (also known as Ephrin A4, Ephrin-A4, EPH-Related Receptor Tyrosine Kinase Ligand 4, LERK-4, EPLG4, LERK4, Eph-Related Receptor Tyrosine Kinase Ligand 4, Ligand Of Eph-Related Kinase 4, EFL4) is the ligand of EPH family EFNA4 mainly expresses in the spleen, lymph nodes, ovary, small intestine, and colon of adults, as well as in the heart, lungs, liver, and kidneys of the fetus. EFNA4 is also highly expressed in hepatocellular carcinoma and correlated with poor prognosis (Lin et al., Molecular Therapy Nucleic Acids, online June 2021). The amino acid sequence of human EFNA4 is as follows (UniProt Accession No. P52798 canonical sequence P52798-1):

(SEQ ID NO: 19)
MRLLPLLRTVLWAAFLGSPLRGGSSLRHVVYWNSSNPRLLRGDAVVEL
GLNDYLDIVCPHYEGPGPPEGPETFALYMVDWPGYESCQAEGPRAYKR
WVCSLPFGHVQFSEKIQRFTPFSLGFEFLPGETYYYISVPTPESSGQC
LRLQVSVCCKERKSESAHPVGSPGESGTSGWRGGDTPSPLCLLLLLLL
LILRLLRIL

EPHA2

EPHA2 (also known as EPH Receptor A2, Tyrosine-Protein Kinase Receptor ECK, Ephrin Type-A Receptor 2, EC 2.7.10.1, ECK, Epithelial Cell Receptor Protein Tyrosine Kinase, Soluble EPHA2 Variant 1, Epithelial Cell Kinase, EC 2.7.10, CTRCT6, ARCC2, CTPP1, EphA2, CTPA) is a member of the largest family of receptor tyrosine kinases (RTKs)—the Eph family Eph receptors and their ligands, the ephrins, have been shown to play several key roles in embryonic development, including tissue boundary formation, neural crest cell migration, axon guidance, central nervous system patterning, bone remodeling and vascular organization. In addition, EphA2 plays an important role in lens, kidney, bone, mammary gland and ear development (Park et al., Genes, 4, 334-357; doi:10.3390/genes4030334, 2013). The amino acid sequence of human EPHA2 is as follows (UniProt Accession No. P29317 canonical sequence P29317-1):

(SEQ ID NO: 20)
MELQAARACFALLWGCALAAAAAAQGKEVVLLDFAAAGGELGWLTHPY
GKGWDLMQNIMNDMPIYMYSVCNVMSGDQDNWLRTNWVYRGEAERIFI
ELKFTVRDCNSFPGGASSCKETFNLYYAESDLDYGTNFQKRLFTKIDT
IAPDEITVSSDFEARHVKLNVEERSVGPLTRKGFYLAFQDIGACVALL
SVRVYYKKCPELLQGLAHFPETIAGSDAPSLATVAGTCVDHAVVPPGG
EEPRMHCAVDGEWLVPIGQCLCQAGYEKVEDACQACSPGFFKFEASES
PCLECPEHTLPSPEGATSCECEEGFFRAPQDPASMPCTRPPSAPHYLT
AVGMGAKVELRWTPPQDSGGREDIVYSVTCEQCWPESGECGPCEASVR
YSEPPHGLTRTSVTVSDLEPHMNYTFTVEARNGVSGLVTSRSFRTASV
SINQTEPPKVRLEGRSTTSLSVSWSIPPPQQSRVWKYEVTYRKKGDSN
SYNVRRTEGFSVTLDDLAPDTTYLVQVQALTQEGQGAGSKVHEFQTLS
PEGSGNLAVIGGVAVGVVLLLVLAGVGFFIHRRRKNQRARQSPEDVYF
SKSEQLKPLKTYVDPHTYEDPNQAVLKFTTEIHPSCVTRQKVIGAGEF
GEVYKGMLKTSSGKKEVPVAIKTLKAGYTEKQRVDFLGEAGIMGQFSH
HNIIRLEGVISKYKPMMIITEYMENGALDKFLREKDGEFSVLQLVGML
RGIAAGMKYLANMNYVHRDLAARNILVNSNLVCKVSDFGLSRVLEDDP
EATYTTSGGKIPIRWTAPEAISYRKFTSASDVWSFGIVMWEVMTYGER
PYWELSNHEVMKAINDGFRLPTPMDCPSAIYQLMMQCWQQERARRPKF
ADIVSILDKLIRAPDSLKTLADFDPRVSIRLPSTSGSEGVPFRTVSEW
LESIKMQQYTEHFMAAGYTAIEKVVQMTNDDIKRIGVRLPGHQKRIAY
SLLGLKDQVNTVGIPI

GFR-alpha-1

GFR-alpha-1 (also known as GDNF Family Receptor Alpha 1, TGF-Beta-Related Neurotrophic Factor Receptor 1, GDNF Family Receptor Alpha-1, GDNFR-Alpha-1, RET Ligand 1, GDNFRA, RETL1, TRNR1, Glial Cell Line-Derived Neurotrophic Factor Receptor Alpha, PI-Linked Cell-Surface Accessory Protein, GPI-Linked Anchor Protein, GDNF Receptor Alpha-1, GDNFR, RET1L) encodes a glycosylphosphatidylinositol (GPI)-linked cell surface receptor for both glial cell line-derived neurotrophic factor (GDNF) and neurturin (NTN), and mediates activation of the RET tyrosine kinase receptor. GFR-alpha-1 is potentially associated with for Hirschsprung disease (Konishi et al., J Neurosci., 34(39): 13127-13138, 2014). The amino acid sequence of human GFR-alpha-1 is as follows (UniProt Accession No. P56159 canonical sequence P56159-1):

(SEQ ID NO: 21)
MFLATLYFALPLLDLLLSAEVSGGDRLDCVKASDQCLKEQSCSTKYRT
LRQCVAGKETNFSLASGLEAKDECRSAMEALKQKSLYNCRCKRGMKKE
GNDLLEDSPYEPVNSRLSDIFRVVPFISDVFQQVEHIPKGNNCLDAAK
KNCLRIYWSMYQSLQACNLDDICKKYRSAYITPCTTSVSNDVCNRRKC
HKALRQFFDKVPAKHSYGMLFCSCRDIACTERRRQTIVPVCSYEEREK
PNCLNLQDSCKTNYICRSRLADFFTNCQPESRSVSSCLKENYADCLLA
YSGLIGTVMTPNYIDSSSLSVAPWCDCSNSGNDLEECLKFLNFFKDNT
CLKNAIQAFGNGSDVTVWQPAFPVQTTTATTTTALRVKNKPLGPAGSE
NEIPTHVLPPCANLQAQKLKSNVSGNTHLCISNGNYEKEGLGASSHIT
TKSMAAPPSCGLSPLLVLVVTALSTLLSLTETS

KIM1

KIM1 (also known as Hepatitis A Virus Cellular Receptor 1, Kidney Injury Molecule 1, T-Cell Immunoglobulin Mucin Family Member 1, T-Cell Immunoglobulin Mucin Receptor 1, T-Cell Membrane Protein 1, TIMD-1, KIM-1, TIM-1, TIMD1, TIM1, TIM, T-Cell Immunoglobulin And Mucin Domain-Containing Protein 1, T Cell Immunoglobin Domain And Mucin Domain Protein 1, CD365 Antigen, HAVCR-1, HAVcr-1, CD365, HAVCR) is a transmembrane protein that is upregulated in renal tubular cells after ischemic injury. KIM1 is not expressed in the normal kidney but is expressed in a variety of human kidney diseases, predominantly in the apical membrane of proximal tubular cells (e.g., Griffin et al, Am J Nephrol. 51(6): 473-479, 2020). The amino acid sequence of human KIM1 is as follows (UniProt Accession No. Q96D42):

(SEQ ID NO: 22)
MHPQVVILSLILHLADSVAGSVKVGGEAGPSVTLPCHYSGAVTSMCWN
RGSCSLFTCQNGIVWTNGTHVTYRKDTRYKLLGDLSRRDVSLTIENTA
VSDSGVYCCRVEHRGWFNDMKITVSLEIVPPKVTTTPIVTTVPTVTTV
RTSTTVPTTTTVPMTTVPTTTVPTTMSIPTTTTVLTTMTVSTTTSVPT
TTSIPTTTSVPVTTTVSTFVPPMPLPRQNHEPVATSPSSPQPAETHPT
TLQGAIRREPTSSPLYSYTTDGNDTVTESSDGLWNNNQTQLFLEHSLL
TANTTKGIYAGVCISVLVLLALLGVIIAKKYFFKKEVQQLSVSFSSLQ
IKALQNAVEKEVQAEDNIYIENSLYATD

PI3

PI3 (also known as Peptidase Inhibitor 3, Skin-Derived Antileukoproteinase, Protease Inhibitor 3, Skin-Derived (SKALP), WAP Four-Disulfide Core Domain Protein 14, Peptidase Inhibitor 3, Skin-Derived, Elastase-Specific Inhibitor, Protease Inhibitor WAP3, Trappin-2, Elafin, WFDC14, SKALP, PI-3, WAP3, ESI, WAP Four-Disulfide Core Domain 14, Pre-Elafin, Cementoin), is an epithelial protein that is secreted by keratinocytes in response to IL-1 and TNFα. PI3 is also shown to be protective against several lung and cardiovascular injuries, including hypoxia-induced pulmonary hypertension, viral myocarditis, and vascular injury in elafin-overexpressing transgenic mice (Li et al., Journal of Interferon & Cytokine Research Vol. 40, No. 6, 2020). The amino acid sequence of human PI3 is as follows (UniProt Accession No. P19957):

(SEQ ID NO: 23)
MRASSFLIVVVFLIAGTLVLEAAVTGVPVKGQDTVKGRVPFNGQDPVK
GQVSVKGQDKVKAQEPVKGPVSTKPGSCPIILIRCAMLNPPNRCLKDT
DCPGIKKCCEGSCGMACFVPQ

TNFRSF11A

TNFRSF11A (also known as TNF Receptor Superfamily Member 11a, Tumor Necrosis Factor Receptor Superfamily Member 11A, Tumor Necrosis Factor Receptor Superfamily, Member 11a, NFKB Activator, Loss Of Heterozygosity, 18, Chromosomal Region 1, Osteoclast Differentiation Factor Receptor, Receptor Activator Of NF-KB, Paget Disease Of Bone 2, ODFR, RANK, Receptor Activator Of Nuclear Factor-Kappa B, CD265 Antigen, LOH18CR1, TRANCER, CD265, OPTB7, OSTS, PDB2, FEO, OFE) is a member of the tumor necrosis factor receptor superfamily (TNFRSF). TNFRSF11A and its ligand are important regulators of the interaction between T cells and dendritic cells. Further, TNFRSF11A is also an essential mediator for osteoclast and lymph node development. The amino acid sequence of human TNFRSF11A is as follows (UniProt Accession No. Q9Y6Q6 canonical sequence Q9Y6Q6-1):

(SEQ ID NO: 24)
MAPRARRRRPLFALLLLCALLARLQVALQIAPPCTSEKHYEHLGRCCN
KCEPGKYMSSKCTTTSDSVCLPCGPDEYLDSWNEEDKCLLHKVCDTGK
ALVAVVAGNSTTPRRCACTAGYHWSQDCECCRRNTECAPGLGAQHPLQ
LNKDTVCKPCLAGYFSDAFSSTDKCRPWTNCTFLGKRVEHHGTEKSDA
VCSSSLPARKPPNEPHVYLPGLIILLLFASVALVAAIIFGVCYRKKGK
ALTANLWHWINEACGRLSGDKESSGDSCVSTHTANFGQQGACEGVLLL
TLEEKTFPEDMCYPDQGGVCQGTCVGGGPYAQGEDARMLSLVSKTEIE
EDSFRQMPTEDEYMDRPSQPTDQLLFLTEPGSKSTPPFSEPLEVGEND
SLSQCFTGTQSTVGSESCNCTEPLCRTDWTPMSSENYLQKEVDSGHCP
HWAASPSPNWADVCTGCRNPPGEDCEPLVGSPKRGPLPQCAYGMGLPP
EEEASRTEARDQPEDGADGRLPSSARAGAGSGSSPGGQSPASGNVTGN
SNSTFISSGQVMNFKGDIIVVYVSQTSQEGAAAAAEPMGRPVQEETLA
RRDSFAGNGPRFPDPCGGPEGLREPEKASRPVQEQGGAKA

CLM1

CLM1 (also known as CD300 Molecule Like Family Member F, Immune Receptor Expressed On Myeloid Cells 1, Immunoglobulin Superfamily Member 13, CMRF35-Like Molecule 1, NK Inhibitory Receptor, IREM-1, IgSF13, CLM-1, IREM1, NKIR, Immunoglobin Superfamily Member 13, Inhibitory Receptor IREM1, CD300f Antigen, CD300F, IGSF13, LMIR3) is a membrane receptor involved in the regulation of immune response. CLM1 is a cell surface glycoprotein with a single IgV-like extracellular domain. CLM1 has biased expression in spleen, bone marrow and 10 other tissues. Recent studies have found CLM1 is associated with major depressive disorder and decreased microglial metabolic fitness (Lago et al., Proc Natl Acad Sci USA.; 117(12): 6651-6662, 2020). The amino acid sequence of human CLM1 is as follows (UniProt Accession No. Q8TDQ1 canonical sequence Q8TDQ1-1):

(SEQ ID NO: 25)
MPLLTLYLLLFWLSGYSIVTQITGPTTVNGLERGSLTVQCVYRSGWET
YLKWWCRGAIWRDCKILVKTSGSEQEVKRDRVSIKDNQKNRTFTVTME
DLMKTDADTYWCGIEKTGNDLGVTVQVTIDPAPVTQEETSSSPTLTGH
HLDNRHKLLKLSVLLPLIFTILLLLLVAASLLAWRMMKYQQKAAGMSP
EQVLQPLEGDLCYADLTLQLAGTSPQKATTKLSSAQVDQVEVEYVTMA
SLPKEDISYASLTLGAEDQEPTYCNMGHLSSHLPGRGPEEPTEYSTIS
RP

TNFRSF12A

TNFRSF12A (also known as TNF Receptor Superfamily Member 12A, Fibroblast Growth Factor-Inducible Immediate-Early Response Protein 14, Tumor Necrosis Factor Receptor Superfamily Member 12A, FGF-Inducible 14, Tweak-Receptor, FN14, type I Transmembrane Protein Fn14, CD266 Antigen, TWEAKR, CD266) is the smallest member of the TNF superfamily of receptors that lacks the cytoplasmic death domain, and has been reported to be elevated in various types of cancer, including HCC. TNFRSF12A is ubiquitously expression in different tissues (Wang et al., Molecular Medicine Reports, Volume 15 Issue 3, 1172-1178, 2017). The amino acid sequence of human TNFRSF12A is as follows (UniProt Accession No. Q9NP84 canonical sequence Q9NP84-1):

(SEQ ID NO: 26)
MARGSLRRLLRLLVLGLWLALLRSVAGEQAPGTAPCSRGSSWSADLDK
CMDCASCRARPHSDFCLGCAAAPPAPFRLLWPILGGALSLTFVLGLLS
GFLVWRRCRRREKFTTPIEETGGEGCPAVALIQ

TRAIL-R2

TRAIL-R2 (also known as TNF Receptor Superfamily Member 10b, Tumor Necrosis Factor Receptor Superfamily, Member 10b, TNF-Related Apoptosis-Inducing Ligand Receptor 2, Death Receptor 5, TRAILR2, KILLER, TRICK2, ZTNFR9, DR5, P53-Regulated DNA Damage-Inducible Cell Death Receptor (Killer), Tumor Necrosis Factor Receptor-Like Protein ZTNFR9, Death Domain Containing Receptor For TRAIL/Apo-2L, Apoptosis Inducing Protein TRICK2A/2B, Apoptosis Inducing Receptor TRAIL-R2, Cytotoxic TRAIL Receptor-2, Fas-Like Protein, TRAIL Receptor 2, CD262 Antigen, KILLER/DR5, TRICK2A, TRICK2B, TRICKB, CD262) is a member of the TNF-receptor superfamily, and contains an intracellular death domain. TRAIL-R2 contribute to induction of apoptosis in a caspase-3-dependent manner in primary human B cells (Staniek et al., Front Immunol.; 10: 951, 2019). The amino acid sequence of human TRAIL-R2 is as follows (UniProt Accession No. 014763 canonical sequence 014763-1):

(SEQ ID NO: 27)
MEQRGQNAPAASGARKRHGPGPREARGARPGPRVPKTLVLVVAAVLLL
VSAESALITQQDLAPQQRAAPQQKRSSPSEGLCPPGHHISEDGRDCIS
CKYGQDYSTHWNDLLFCLRCTRCDSGEVELSPCTTTRNTVCQCEEGTF
REEDSPEMCRKCRTGCPRGMVKVGDCTPWSDIECVHKESGTKHSGEVP
AVEETVTSSPGTPASPCSLSGIIIGVTVAAVVLIVAVFVCKSLLWKKV
LPYLKGICSGGGGDPERVDRSSQRPGAEDNVLNEIVSILQPTQVPEQE
MEVQEPAEPTGVNMLSPGESEHLLEPAEAERSQRRRLLVPANEGDPTE
TLRQCFDDFADLVPFDSWEPLMRKLGLMDNEIKVAKAEAAGHRDTLYT
MLIKWVNKTGRDASVHTLLDALETLGERLAKQKIEDHLLSSGKFMYLE
GNADSAMS

RGMB

RGMB (also known as Repulsive Guidance Molecule BMP Co-Receptor B, DRG11-Responsive Axonal Guidance And Outgrowth Of Neurite, Repulsive Guidance Molecule Family Member B, RGM Domain Family, Member B, DRAGON, Repulsive Guidance Molecule B) is a repulsive guidance molecule, which is glycosylphosphatidylinositol (GPI)-anchored cell surface glycoprotein coreceptor for BMP/GDF morphogens and can potentiate signaling of at least BMP2 and BMP6. RGMB can physically bridge NEO1 and BMP2, suggesting a functional link between these two pathways, and contributes to the patterning of the developing nervous system (Malinauskas et al., Proc Natl Acad Sci USA., 117(27): 15620-15631, 2020). The amino acid sequence of human RGMB is as follows (UniProt Accession No. Q6NW40):

(SEQ ID NO: 28)
MGLRAAPSSAAAAAAEVEQRRSPGLCPPPLELLLLLLFSLGLLHAGDC
QQPAQCRIQKCTTDFVSLTSHLNSAVDGFDSEFCKALRAYAGCTQRTS
KACRGNLVYHSAVLGISDLMSQRNCSKDGPTSSTNPEVTHDPCNYHSH
AGAREHRRGDQNPPSYLFCGLFGDPHLRTFKDNFQTCKVEGAWPLIDN
NYLSVQVTNVPVVPGSSATATNKITIIFKAHHECTDQKVYQAVTDDLP
AAFVDGTTSGGDSDAKSLRIVERESGHYVEMHARYIGTTVFVRQVGRY
LTLAIRMPEDLAMSYEESQDLQLCVNGCPLSERIDDGQGQVSAILGHS
LPRTSLVQAWPGYTLETANTQCHEKMPVKDIYFQSCVFDLLTTGDANF
TAAAHSALEDVEALHPRKERWHIFPSSGNGTPRGGSDLSVSLGLTCLI
LIVFL

DKK4

DKK4 (also known as Dickkopf WNT Signaling Pathway Inhibitor 4, Dickkopf-Related Protein 4, Dickkopf-4, HDkk-4, Dickkopf (Xenopus Laevis) Homolog 4, Dickkopf Homolog 4 (Xenopus Laevis), DKK-4) is a member of the Wnt/β-catenin pathway, which is an important pathway involved in the development of multiple tissues, such as the regulation of bone formation. DKK4 may have an important role in osteogenesis (Sun et al., Biochemical and Biophysical Research Communications, Volume 516, Issue 1, Pages 171-176, 2019). The amino acid sequence of human DKK4 is as follows (UniProt Accession No. Q9UBT3):

(SEQ ID NO: 29)
MVAAVLLGLSWLCSPLGALVLDFNNIRSSADLHGARKGSQCLSDTDCN
TRKFCLQPRDEKPFCATCRGLRRRCQRDAMCCPGTLCVNDVCTTMEDA
TPILERQLDEQDGTHAEGTTGHPVQENQPKRKPSIKKSQGRKGQEGES
CLRTFDCGPGLCCARHFWTKICKPVLLEGQVCSRRGHKDTAQAPEIFQ
RCDCGPGLLCRSQLTSNRQHARLRVCQKIEKL

TFF3

TFF3 (also known as Trefoil Factor 3, Trefoil Factor 3 (Intestinal), Polypeptide P1.B, ITF, TFI, Intestinal Trefoil Factor, HP1.B, HITF, P1B) is a member of the trefoil family which is characterized by having at least one copy of the trefoil motif, a 40-amino acid domain that contains three conserved disulfides. TFF3 is disulfide-linked homo-dimers with each monomer possessing a single trefoil domain. Abnormally expressed TFF3 is involved in the progression of cancers, which accelerates the oncogenic characteristics of prostate cancer cells and diminishes sensitivity to radiation (Wu et al., J Cell Mol Med., 24(15): 8589-8602, 2020). The amino acid sequence of human TFF3 is as follows (UniProt Accession No. Q07654):

(SEQ ID NO: 30)
MKRVLSCVPEPTVVMAARALCMLGLVLALLSSSSAEEYVGLSANQCAV
PAKDRVDCGYPHVTPKECNNRGCCFDSRIPGVPWCFKPLQEAECTF

CRELD2

CRELD2 (also known as Cysteine Rich With EGF Like Domains 2, Cysteine-Rich With EGF-Like Domain Protein 2, Protein Disulfide Isomerase CRELD2, Cysteine-Rich With EGF-Like Domains 2, EC 5.3.4.1) is an ER stress-inducible gene. It is an about 50 kDa secretory glycoprotein that predominantly localizes to the ER and Golgi apparatus. Its promoter region, which is well conserved among various species, contains a typical ER stress response element (ERSE; CGTGG-N9-ATTGG) that is positively regulated by the ER stress master regulator ATF6. It has shown that CRELD2 is upregulated and secreted following ER retention of mutant cartilage extracellular matrix proteins in mouse knock-in models of chondrodysplasia resulting from mutations in matrilin-3 (Matn3) or type X collagen (Co110a1) (Kim et al., JCI Insight, 2(23): e92896, 2017). The amino acid sequence of human CRELD2 is follows (UniProt Accession No. Q6UXH1 canonical sequence Q6UXH1-1):

(SEQ ID NO: 31)
MRLPRRAALGLLPLLLLLPPAPEAAKKPTPCHRCRGLVDKFNQGMVDT
AKKNFGGGNTAWEEKTLSKYESSEIRLLEILEGLCESSDFECNQMLEA
QEEHLEAWWLQLKSEYPDLFEWFCVKTLKVCCSPGTYGPDCLACQGGS
QRPCSGNGHCSGDGSRQGDGSCRCHMGYQGPLCTDCMDGYFSSLRNET
HSICTACDESCKTCSGLTNRDCGECEVGWVLDEGACVDVDECAAEPPP
CSAAQFCKNANGSYTCEECDSSCVGCTGEGPGNCKECISGYAREHGQC
ADVDECSLAEKTCVRKNENCYNTPGSYVCVCPDGFEETEDACVPPAEA
EATEGESPTQLPSREDL

CADM3

CADM3 (also known as Cell Adhesion Molecule 3, Immunoglobulin Superfamily Member 4B, Synaptic Cell Adhesion Molecule 3, Brain Immunoglobulin Receptor, SynCAM3, IGSF4B, NECL1, TSLL1, Dendritic Cell Nectin-Like Protein 1 Short Isoform, Immunoglobulin Superfamily, Member 4B, Nectin-Like Protein 1, TSLC1-Like Protein 1, Nectin-Like 1, TSLC1-Like 1, SYNCAM3, Ned-1, IgSF4B, NECL-1, BIgR) is a calcium-independent cell-cell adhesion protein that can form homodimers or heterodimers with other nectin proteins. It is reported that CADM3 has tumor suppressing activity (Rebelo et al., Brain, Volume 144, Issue 4, 1197-1213, 2021). The amino acid sequence of human CADM3 is as follows (UniProt Accession No. Q8N126 canonical sequence Q8N126-1):

(SEQ ID NO: 32)
MGAPAASLLLLLLLFACCWAPGGANLSQDDSQPWTSDETVVAGGTVVL
KCQVKDHEDSSLQWSNPAQQTLYFGEKRALRDNRIQLVTSTPHELSIS
ISNVALADEGEYTCSIFTMPVRTAKSLVTVLGIPQKPIITGYKSSLRE
KDTATLNCQSSGSKPAARLTWRKGDQELHGEPTRIQEDPNGKTFTVSS
SVTFQVTREDDGASIVCSVNHESLKGADRSTSQRIEVLYTPTAMIRPD
PPHPREGQKLLLHCEGRGNPVPQQYLWEKEGSVPPLKMTQESALIFPF
LNKSDSGTYGCTATSNMGSYKAYYTLNVNDPSPVPSSSSTYHAIIGGI
VAFIVFLLLIMLIFLGHYLIRHKGTYLTHEAKGSDDAPDADTAIINAE
GGQSGGDDKKEYFI

ADAM22

ADAM22 (also known as ADAM Metallopeptidase Domain 22, Metalloproteinase-Like, Disintegrin-Like, And Cysteine-Rich Protein 2, Disintegrin And Metalloproteinase Domain-Containing Protein 22, A Disintegrin And Metalloproteinase Domain 22, Metalloproteinase-Disintegrin ADAM22-3, ADAM 22, MDC2, EIEE61) is a receptor on the surface of the postsynaptic neuron to regulate signal transmission through binding to leucine-rich, glioma inactivated gene 1 (LGI1), a neuronal protein and a specific ligand for ADAM22. Unlike other family members implicated in cancer, ADAM22 lacks a functional metalloproteinase domain and may mediate its pro-tumourigenic effects through interaction with other cell surface tyrosine kinase receptors using its EGF-like domain (Charmsaz et al., BMC Med.; 18: 349, 2020). The amino acid sequence of human ADAM22 is as follows (UniProt Accession No. Q9P0K1 canonical sequence Q9P0K1-1):

(SEQ ID NO: 33)
MQAAVAVSVPFLLLCVLGTCPPARCGQAGDASLMELEKRKENRFVERQ
SIVPLRLIYRSGGEDESRHDALDTRVRGDLGGPQLTHVDQASFQVDAF
GTSFILDVVLNHDLLSSEYIERHIEHGGKTVEVKGGEHCYYQGHIRGN
PDSFVALSTCHGLHGMFYDGNHTYLIEPEENDTTQEDFHFHSVYKSRL
FEFSLDDLPSEFQQVNITPSKFILKPRPKRSKRQLRRYPRNVEEETKY
IELMIVNDHLMFKKHRLSVVHTNTYAKSVVNMADLIYKDQLKTRIVLV
AMETWATDNKFAISENPLITLREFMKYRRDFIKEKSDAVHLFSGSQFE
SSRSGAAYIGGICSLLKGGGVNEFGKTDLMAVTLAQSLAHNIGIISDK
RKLASGECKCEDTWSGCIMGDTGYYLPKKFTQCNIEEYHDFLNSGGGA
CLFNKPSKLLDPPECGNGFIETGEECDCGTPAECVLEGAECCKKCTLT
QDSQCSDGLCCKKCKFQPMGTVCREAVNDCDIRETCSGNSSQCAPNIH
KMDGYSCDGVQGICFGGRCKTRDRQCKYIWGQKVTASDKYCYEKLNIE
GTEKGNCGKDKDTWIQCNKRDVLCGYLLCTNIGNIPRLGELDGEITST
LVVQQGRTLNCSGGHVKLEEDVDLGYVEDGTPCGPQMMCLEHRCLPVA
SFNFSTCLSSKEGTICSGNGVCSNELKCVCNRHWIGSDCNTYFPHNDD
AKTGITLSGNGVAGTNIIIGIIAGTILVLALILGITAWGYKNYREQRQ
LPQGDYVKKPGDGDSFYSDIPPGVSTNSASSSKKRSNGLSHSWSERIP
DTKHISDICENGRPRSNSWQGNLGGNKKKIRGKRFRPRSNSTETLSPA
KSPSSSTGSIASSRKYPYPMPPLPDEDKKVNRQSARLWETSI

In certain embodiments, the biomarkers are selected from any one of (or a combination of, or combinations of) TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In other embodiments, the biomarkers include at least two biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least three biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least four biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least five biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least six biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least seven biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least eight biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least nine biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least ten biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least eleven biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least twelve biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least thirteen biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least fourteen biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least fifteen biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least sixteen biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least seventeen biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least eighteen biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least nineteen biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least twenty biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least twenty-one biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least twenty-two biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least twenty-three biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least twenty-four biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least twenty-five biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least twenty-six biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least twenty-seven biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least twenty-eight biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least twenty-nine biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least thirty or more biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22. In another embodiment, the biomarkers include at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty one, at least twenty two, at least twenty three, at least twenty four, at least twenty five, at least twenty six, at least twenty seven, at least twenty eight, at least twenty nine, at least thirty or more biomarkers selected from the group consisting of the TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR alpha 1, KIM1, TNFRSF11A, CLM1, TNFRSF12A, TRAIL-R2, RGMB, DKK4, TFF3, CRELD2, CADM3, and ADAM22.

For subjects with diabetes, the risk of chronic kidney disease and ESKD remains relatively high despite improvements in glycemic control and advances in reno-protective therapies over the last 20 years for the prevention and treatment of DKD (Rosolowsky et al., J Am Soc Nephrol 22: 545-553 (2011); de Boer et al., JAMA 305: 2532-2539 (2011)). Findings from Joslin Kidney Study, a longitudinal study of more than 3000 subjects with diabetes, demonstrate that progressive renal decline is the major clinical manifestation of DKD that underlies progression to ESKD (Perkins et al., N Engl J Med 348: 2285-2293 (2003); Perkins et al., J Am Soc Nephrol 18: 1353-1361 (2007); Krolewski, Diabetes Care 38, 954-962 (2015); Krolewski et al., Kidney International 91: 1300-1311 (2017)).

The incidence of ESKD in diabetes patients continues to increase despite improvements in glycemic control and advances in reno-protective therapies, which are almost universally implemented.

In one embodiment, a subject having diabetes who is at risk of renal decline and/or is at risk of developing ESRD may be identified by determining the relative level of a biomarker, or a group of biomarkers, in a sample from the subject, wherein the biomarker, or group of biomarkers, is at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, at least twenty-seven, at least twenty-eight, at least twenty-nine, at least thirty or more biomarkers selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22.

In one embodiment, a subject having diabetes who is at risk of renal decline may be identified by determining the relative level of a biomarker, or a group of biomarkers, in a sample from the subject, wherein the biomarker, or group of biomarkers, is at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty or more biomarkers selected from the group consisting of TNF-R1, TNF-R2, CD27, LTBR, TNF-RSF6B, TNF-RSF10A, TNF-RSF4, EDA2R, RELT, CD160, IL-1RT1, DLL1, LAYN, MMP7, NBL1, PI3, WFDC2, EFNA4, EPHA2, GFR-alpha-1, and KIM1.

In one embodiment, a subject having diabetes who is at risk of developing ESRD may be identified by determining the relative level of a biomarker, or a group of biomarkers, in a sample from the subject, wherein the biomarker, or group of biomarkers, is at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, or more biomarkers selected from the group consisting of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, and KIM1. Determining whether a level of a biomarker in a biological sample derived from a “test” subject is different from the level of the biomarker present in a control (e.g., non-disease control) subject may be ascertained by comparing the level of the biomarker in the sample from the “test” subject with a suitable control of the same biomarker. The skilled person can select an appropriate control for the assay in question. For example, a suitable control may be a biological sample derived from a known subject, e.g., a normoalbuminuric control, or e.g., a non-decliner, a non-progressor, or a healthy control level, or a standard control level, or a non-T1D or a non-T2D control subject.

In one embodiment, in determining whether a subject has, or is at risk of developing, renal decline and has elevated levels of biomarkers associated with risk of renal decline, a statistically significant increase in the level of at least one biomarker in a sample from the subject relative to the suitable control is indicative that the subject has, or is at risk of developing, renal decline. Alternatively, if a suitable control is obtained from a subject known to have renal decline, levels of the at least one biomarker comparable to such a control are indicative of a subject having renal decline or at risk of developing renal decline.

In another embodiment, in determining whether a subject has, or is at risk of developing, ESRD and/or has elevated levels of biomarkers associated with progression to ESRD, a statistically significant increase in the level of at least one biomarker in a sample from the subject relative to the suitable control is indicative that the subject has, or is at risk of developing, ESRD. Alternatively, if a suitable control is obtained from a subject known to have ESRD, levels comparable to such a control are indicative of a risk of developing ESRD.

In one embodiment, in determining whether a subject is a non-decliner (i.e., a subject who has a reduced risk of developing renal decline) and has levels of biomarkers that are inconsistent with renal decline and/or developing renal decline, a comparable level of at least one biomarker in a sample from the subject relative to the suitable control, e.g., a non-decliner, a non-T1D, a non-T2D control, a normoalbuminuric control, a healthy control level, or a standard control level, is indicative that the subject has a reduced risk of renal decline (i.e., is a non-decliner). Alternatively, if a suitable control is obtained from a subject known to be a non-decliner, levels comparable to such a control are indicative of a reduced risk of renal decline (i.e., is a non-decliner).

In one embodiment, in determining whether a subject is a non-progressor (i.e., a subject who has a reduced risk of developing ESRD) and has levels of biomarkers that are inconsistent with progression to ESRD, a comparable level of at least one biomarker in a sample from the subject relative to the suitable control, e.g., a non-progressor, a non-T1D, a non-T2D control, a normoalbuminuric control, a healthy control level, or a standard control level, is indicative that the subject has a reduced risk of ESRD (i.e., a non-progressor). Alternatively, if a suitable control is obtained from a subject known to be a non-progressor, levels comparable to such a control are indicative of a reduced risk of progression to ESRD (i.e., a non-progressor).

Generally, a suitable control may also be a reference standard. A reference standard serves as a reference level for comparison, such that test samples can be compared to the reference standard in order to infer the renal decline status and/or ESRD status of a subject. A reference standard may be representative of the level of one or more protein biomarkers in a known subject, e.g., a subject known to be a normal subject (e.g., a healthy subject without T1D or T2D), or a subject known to have renal decline and/or ESRD. Likewise, a reference standard may be representative of the level of one or more protein biomarkers in a population of known subjects, e.g., a population of subjects known to be normal subjects, or a population of subjects known to have renal decline and/or ESRD or known to be at a risk of developing renal decline and/or ESRD. The reference standard may be obtained, for example, by pooling samples from a plurality of individuals and determining the level of a protein biomarker in the pooled samples, to thereby produce a standard over an averaged population. Such a reference standard represents an average level of a protein biomarker among a population of individuals. A reference standard may also be obtained, for example, by averaging the level of a protein biomarker determined to be present in individual samples obtained from a plurality of individuals. Such a standard is also representative of an average level of a protein biomarker among a population of individuals. A reference standard may also be a collection of values each representing the level of a protein biomarker in a known subject in a population of individuals. In certain embodiments, test samples may be compared against such a collection of values in order to infer the risk status of a subject. In certain embodiments, the reference standard is an absolute value. In such embodiments, test samples may be compared against the absolute value in order to infer the risk status of a subject. In one embodiment, a comparison between the level of at least one protein biomarker in a sample relative to a suitable control is made by executing a software classification algorithm. The skilled person can readily envision additional suitable controls that may be appropriate depending on the assay in question. The aforementioned suitable controls are exemplary, and are not intended to be limiting.

In certain aspects, the present disclosure features a method for identifying a subject who has, or is at risk of developing, renal decline, said method comprising determining the relative level of a decliner biomarker in a sample (e.g., a urine sample or a plasma sample) from the subject, wherein a higher level of the decliner biomarker in the sample relative to a non-decliner control level of the decliner biomarker (or a normoalbuminuric, or a healthy control level, or a standard control level, or non-T1D/non-T2D control level of the decliner biomarker) indicates that the subject has, or is at risk of developing, renal decline. In other aspects, the present invention features a method for identifying a subject who has a reduced risk for developing renal decline, the method comprising determining the relative level of a decliner biomarker in a sample (e.g., a urine sample or a plasma sample) from the subject, wherein a lower level of the decliner biomarker in the sample relative to a decliner control level of the decliner biomarker or a comparable level of the decliner biomarker to a normoalbuminuric (or non-T1D/non-T2D) control level of the decliner biomarker indicates that the subject does not have, or is not at risk of developing renal decline.

In certain aspects, the present invention features a method for identifying a subject who has, or is at risk of developing, ESRD, said method comprising determining the relative level of a progressor biomarker in a sample (e.g., a urine sample or a plasma sample) from the subject, wherein a higher level of the progressor biomarker in the sample relative to a non-progressor control level of the progressor biomarker (or a normoalbuminuric, or a healthy control level, or a standard control level, or non-T1D/non-T2D control level of the progressor biomarker) indicates that the subject has, or is at risk of developing, ESRD. In other aspects, the present invention features a method for identifying a subject who has a reduced risk for developing ESRD, the method comprising determining the relative level of a progressor biomarker in a sample (e.g., a urine sample or a plasma sample) from the subject, wherein a lower level of the progressor biomarker in the sample relative to a progressor control level of the progressor biomarker or a comparable level of the progressor biomarker to a normoalbuminuric (or non-T1D/non-T2D) control level of the progressor biomarker indicates that the subject does not have, or is not at risk of developing ESRD.

In one embodiment, the method comprises determining the relative level of at least two decliner biomarkers in a sample (e.g., a urine sample or a plasma sample) from the subject, and determining the relative level of at least two decliner biomarkers in a sample (e.g., a plasma sample) from a known non-decliner subject (control) (or from a known normoalbuminuric subject, or from a known a healthy control subject, or from a known non-T1D/non-T2D subject). In certain embodiments, the at least two decliner biomarkers are selected from the group consisting of TNF-R1, TNF-R2, CD27, LTBR, TNF-RSF6B, TNF-RSF10A, TNF-RSF4, EDA2R, RELT, CD160, IL-1RT1, DLL1, LAYN, MMP7, NBL1, PI3, WFDC2, EFNA4, EPHA2, GFR-alpha-1, and KIM1.

In one embodiment, the method comprises determining the relative level of at least two progressor biomarkers in a sample (e.g., a urine sample or a plasma sample) from the subject, and determining the relative level of at least two progressor biomarkers in a sample (e.g., a urine sample or a plasma sample) from a known non-progressor subject (control) (or from a known normoalbuminuric subject, or from a known a healthy control subject, or from a known non-T1D/non-T2D subject). In certain embodiments, the at least two progressor biomarkers are selected from the group consisting of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, and KIM1.

Table 5 (and Table 6) provides a list of protein biomarkers identified in the methods of the present disclosure.

Other protein biomarkers that may be used to determine risk of renal decline and/or risk of ESRD include any one or more of TNFRSF11A, CLM1, TNFRSF12A, TRAIL-R2, RGMB, DKK4, TFF3, CRELD2, CADM3, and ADAM22. In one embodiment, any one or more of TNFRSF11A, CLM1, TNFRSF12A, TRAIL-R2, RGMB, DKK4, TFF3, CRELD2, CADM3, and ADAM22 is used to predict progression to ESRD during 7-15 years of follow-up. In another embodiment, a protein biomarker that may be used to determine risk of renal decline and/or risk of ESRD includes CST3.

The protein biomarkers described herein can be used individually or in combination in methods to identify (e.g. diagnostic tests) a risk renal decline and/or a risk of developing ESRD in a subject. The methods also include monitoring renal decline and/or the course of progression to ESRD. Based on the risk of progression to ESRD in a subject, additional procedures may be indicated, including, for example, additional diagnostic tests or therapeutic procedures, or additional markers.

Other markers contemplated by the disclosure that may be used in combination the protein biomarkers disclosed herein for methods to identify (e.g. diagnostic tests) a risk renal decline or a risk of developing ESRD in a subject include, but are not limited to, ACR (albumin-to-creatinine ratio), systolic blood pressure (SBP) and eGFR.

Common tests for statistical significance also include, but are not limited to, t-test, ANOVA, Kniskal-Wallis, Wilcoxon, Mann-Whitney, and odds ratio. Protein biomarkers, alone or in combination, can be used to provide a measure of the relative risk that a subject is or is not at risk for progression to ESRD.

The present invention has identified particular biomarkers that are differentially present in subjects who have, or are at risk of developing, renal decline relative to non-decliners and/or have, or are at risk of developing, ESRD relative to non-progressors. The biomarkers disclosed herein are differentially present in biological samples derived from subjects who are decliners or non-decliners, or who are progressors or non-progressors, and thus are individually useful in facilitating the determination of a risk of developing renal decline and/or ESRD in a test subject. Such methods involve determining the level of the biomarker (or combination of biomarkers) in a sample derived from the subject. Determining the level of the biomarker(s) in a sample may include measuring, detecting, or assaying the level of the biomarker(s) in the sample using any suitable method, for example, the methods set forth herein. Determining the level of the biomarker(s) in a sample may also include examining the results of an assay that measured, detected, or assayed the level of the biomarker(s) in the sample. The method may also involve comparing the level of the biomarker(s) in a sample with a suitable control. A change in the level of the biomarker(s) relative to that in a normal subject as assessed using a suitable control is indicative of the risk of renal decline and/or the risk of progression to ESRD of the subject. A diagnostic amount of a biomarker that represents an amount of the biomarker above or below which a subject is classified as having a particular risk status can be used. For example, if the biomarker is downregulated in samples derived from the subject sample as compared to a control sample, a measured amount below the diagnostic cutoff provides an indication of risk of developing renal decline and/or ESRD. Alternatively, if the biomarker is upregulated in samples derived from the subject sample as compared to a control sample, a measured amount above the diagnostic cutoff provides an indication of risk of developing renal decline and/or ESRD.

In some embodiments, the biological sample is obtained from the human subject at a first time point and a second time point. In other embodiments, the second time point is obtained from the human subject about 6 months, about 12 months, about 18 months, about 24 months, about 3 years, about 4 years, about 5 years, about 10 years or about 15 years after the first time point. In certain other embodiments, the method further comprises comparing the level of the at least one marker in the biological sample obtained from the human subject at a first time point to the biological sample obtained from the human subject at a second time point. By determining levels at different time points, renal decline, e.g., risk of ESRD, (or improvement) can be determined.

As is well-understood in the art, adjusting the particular diagnostic cut-off used in an assay allows one to adjust the sensitivity and/or specificity of the diagnostic assay as desired. The particular diagnostic cut-off can be determined, for example, by measuring or detecting the amount of the biomarker(s) in a statistically significant number of samples from subjects with different risk statuses, and drawing the cut-off at the desired level of accuracy, sensitivity, and/or specificity. In certain embodiments, the diagnostic cut-off can be determined with the assistance of an algorithm, as described herein.

Optionally, the method may further comprise providing a diagnosis that the subject is or is not at risk of developing renal decline and/or ESRD based on the level of at least one protein biomarker in a sample (e.g., a biological sample from a human subject). Additionally or alternatively, the method may further comprise correlating a difference in the level or levels of at least one protein biomarker relative to a suitable control with a diagnosis of renal decline and/or ESRD.

While individual protein biomarkers are useful in identifying a subject who is at risk of developing renal decline and/or ESRD, as shown herein, a combination of protein biomarkers may also be used to provide a greater predictive value of risk of developing renal decline and/or ESRD. Specifically, the detection of a plurality (or combination) of protein biomarkers can increase the accuracy, sensitivity, and/or specificity of a diagnostic test. Accordingly, the present invention includes the individual biomarkers described herein, as well as biomarker combinations, and their use in methods, composition and systems described herein. In certain embodiments, the levels of at least two protein biomarkers in the sample are determined, wherein the at least two protein biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, at least thirty or more biomarkers) are selected from TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22.

The level of at least two protein biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight at least twenty-nine biomarkers, or at least thirty or more biomarkers as described herein) indicative of the risk of development of renal decline and/or ESRD may be used as a stand-alone diagnostic indicator of risk in a subject. Optionally, the methods may include the measurement of at least one additional marker (e.g., ACR (albumin-to-creatinine ratio), systolic blood pressure (SBP) and eGFR) or performance of a test to facilitate identifying a subject who is at risk of developing renal decline and/or ESRD. For example, in some embodiments, renal decline and/or ESRD may be diagnosed, in part, using the Glomerular Filtration Rate (GFR) test. Alternatively, in other embodiments, renal decline and/or ESRD can be determined, in part, by measuring estimated Glomerular Filtration Rate (eGFR) in the urine of a subject, wherein an estimated Glomerular Filtration Rate (eGFR) change of at least <−3 ml/min/year indicates renal decline or progression to ESRD.

In one embodiment, the disclosure features a method of diagnosing renal decline (RD) in a patient, said method comprising obtaining a sample (e.g., a urine sample or a plasma sample) from a human patient, detecting the level of at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers) selected from the group consisting of TNF-R1, TNF-R2, CD27, LTBR, TNF-RSF6B, TNF-RSF10A, TNF-RSF4, EDA2R, RELT, CD160, IL-1RT1, DLL1, LAYN, MMP7, NBL1, PI3, WFDC2, EFNA4, EPHA2, GFR-alpha-1, and KIM1 in the sample using, for example, the Slow Off-rate Modified Aptamer (SOMA)scan platform, the OLINK Proximity Extension Assay based proteomic platform, an immunoassay, an ELISA, a western blot, a microarray analysis, a mass spectrometry, a mass spectrometry matrix assisted laser desorption ionization-time-of-flight (MALDI-TOF), an inductively coupled plasma mass spectrometry (ICP-MS), a triggered-by-offset, multiplexed, accurate-mass, high-resolution, and absolute quantification (TOMAHAQ), a direct analysis in real time mass spectrometry (DART-MS), a secondary ion mass spectrometry (SIMS), a liquid chromatography (LC) fractionation, a Mesoscale platform, or electrochemiluminescence detection diagnose a patient with RD when a higher level of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers) is detected in comparison to, for example, a non-decliner control level (or a normoalbuminuric control level, or a non-T1D patient control level, or a non-T2D patient control level) of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers) in the sample.

In one embodiment, the disclosure features a method of diagnosing end-stage renal disease (ESRD) in a patient, said method comprising obtaining a sample (e.g., a urine sample, or a plasma sample) from a human patient, detecting the relative level of at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers) selected from the group consisting of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, and KIM1 in the sample using, for example, the Slow Off-rate Modified Aptamer (SOMA)scan platform, the OLINK Proximity Extension Assay based proteomic platform, an immunoassay, an ELISA, a western blot, a microarray analysis, a mass spectrometry, a mass spectrometry matrix assisted laser desorption ionization-time-of-flight (MALDI-TOF), an inductively coupled plasma mass spectrometry (ICP-MS), a triggered-by-offset, multiplexed, accurate-mass, high-resolution, and absolute quantification (TOMAHAQ), a direct analysis in real time mass spectrometry (DART-MS), a secondary ion mass spectrometry (SIMS), a liquid chromatography (LC) fractionation, a Mesoscale platform, or an electrochemiluminescence detection to diagnose a patient with ESKD when a higher level of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers) is detected in comparison to, for example, a non-progressor control level (or a normoalbuminuric control level, or a healthy control level, or a standard control level, or a non-T1D patient control level, or a non-T2D patient control level) of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers).

In one embodiment, the invention features a method of identifying a subject who is at risk of renal decline, said method comprising obtaining a sample (e.g., a urine sample or a plasma sample) from a human patient, detecting the relative level of at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers) selected from the group consisting of TNF-R1, TNF-R2, CD27, LTBR, TNF-RSF6B, TNF-RSF10A, TNF-RSF4, EDA2R, RELT, CD160, IL-1RT1, DLL1, LAYN, MMP7, NBL1, PI3, WFDC2, EFNA4, EPHA2, GFR-alpha-1, and KIM1 in the sample using, for example, the Slow Off-rate Modified Aptamer (SOMA)scan platform, the OLINK Proximity Extension Assay based proteomic platform, an immunoassay, an ELISA, a western blot, a microarray analysis, a mass spectrometry, a mass spectrometry matrix assisted laser desorption ionization-time-of-flight (MALDI-TOF), an inductively coupled plasma mass spectrometry (ICP-MS), a triggered-by-offset, multiplexed, accurate-mass, high-resolution, and absolute quantification (TOMAHAQ), a direct analysis in real time mass spectrometry (DART-MS), a secondary ion mass spectrometry (SIMS), a liquid chromatography (LC) fractionation, a Mesoscale platform, or an electrochemiluminescence detection, or by microarray analysis, and identifying the subject who is at risk of renal decline when a higher level of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers) is detected in comparison to, for example, a non-decliner control level (or a normoalbuminuric control level, or a healthy control level, or a standard control level, or a non-T1D patient control level, or a non-T2D patient control level) of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers) in the sample.

In another embodiment, the invention features a method of identifying a subject who is at risk of developing ESRD, said method comprising obtaining a sample (e.g., a urine or a plasma sample) from a human patient, detecting the relative level of at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers) selected from the group consisting of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, and KIM1 in the sample using, for example, the Slow Off-rate Modified Aptamer (SOMA)scan platform, the OLINK Proximity Extension Assay based proteomic platform, an immunoassay, an ELISA, a western blot, a microarray analysis, a mass spectrometry, a mass spectrometry matrix assisted laser desorption ionization-time-of-flight (MALDI-TOF), an inductively coupled plasma mass spectrometry (ICP-MS), a triggered-by-offset, multiplexed, accurate-mass, high-resolution, and absolute quantification (TOMAHAQ), a direct analysis in real time mass spectrometry (DART-MS), a secondary ion mass spectrometry (SIMS), a liquid chromatography (LC) fractionation, a Mesoscale platform, an electrochemiluminescence detection, or a microarray analysis, and identifying the subject who is at risk of developing ESKD when a higher level of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers) is detected in comparison to, for example, a non-progressor control level (or a normoalbuminuric control level, or a healthy control level, or a standard control level, or a non-T1D patient control level, or a non-T2D patient control level) of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or more biomarkers) in the sample.

In one embodiment, the invention features a method of identifying a subject who is a non-decliner, said method comprising obtaining a sample (e.g., a urine sample or a plasma sample) from a human patient, detecting the relative level of at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty or more biomarkers) selected from the group consisting of TNF-R1, TNF-R2, CD27, LTBR, TNF-RSF6B, TNF-RSF10A, TNF-RSF4, EDA2R, RELT, CD160, IL-1RT1, DLL1, LAYN, MMP7, NBL1, PI3, WFDC2, EFNA4, EPHA2, GFR-alpha-1, and KIM1 in the sample using, for example, the Slow Off-rate Modified Aptamer (SOMA)scan platform, the OLINK Proximity Extension Assay based proteomic platform, an immunoassay, an ELISA, a western blot, a microarray analysis, a mass spectrometry, a mass spectrometry matrix assisted laser desorption ionization-time-of-flight (MALDI-TOF), an inductively coupled plasma mass spectrometry (ICP-MS), a triggered-by-offset, multiplexed, accurate-mass, high-resolution, and absolute quantification (TOMAHAQ), a direct analysis in real time mass spectrometry (DART-MS), a secondary ion mass spectrometry (SIMS), a liquid chromatography (LC) fractionation, a Mesoscale platform, an electrochemiluminescence detection, or a microarray analysis, and identifying the subject who is a non-decliner when a comparable (or lower) level of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty or more biomarkers) is detected in comparison to, for example, a known non-decliner control level (or a normoalbuminuric control level, or a healthy control level, or a standard control level or a non-T1D patient control level, or a non-T2D patient control level) of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty or more biomarkers) in the sample.

In one embodiment, the invention features a method of identifying a subject who is a non-progressor, said method comprising obtaining a sample (e.g., a urine sample or a plasma sample) from a human patient, detecting the relative level of at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty or more biomarkers) selected from the group consisting of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, and KIM1 in the sample using, for example, the Slow Off-rate Modified Aptamer (SOMA)scan platform, the OLINK Proximity Extension Assay based proteomic platform, an immunoassay, an ELISA, a western blot, a microarray analysis, a mass spectrometry, a mass spectrometry matrix assisted laser desorption ionization-time-of-flight (MALDI-TOF), an inductively coupled plasma mass spectrometry (ICP-MS), a triggered-by-offset, multiplexed, accurate-mass, high-resolution, and absolute quantification (TOMAHAQ), a direct analysis in real time mass spectrometry (DART-MS), a secondary ion mass spectrometry (SIMS), a liquid chromatography (LC) fractionation, a Mesoscale platform, an electrochemiluminescence detection, or by microarray analysis, and identifying the subject who is a non-progressor when a comparable (or lower) level of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty or more biomarkers) is detected in comparison to, for example, a known non-progressor control level (or a normoalbuminuric control level, or a healthy control level, or a standard control level or a non-T1D patient control level, or a non-T2D patient control level) of the at least two biomarkers (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty or more biomarkers) in the sample.

Methods and compositions are based, at least in part, on the discovery that certain biomarkers are associated with renal decline and/or progression to ESRD, for example, in subjects with T1D or T2D. Examples of biomarkers that may be used in the methods and compositions as described herein are provided herein. As described herein, the term biomarker is intended to include the protein, as well as functional fragments thereof. A functional fragment would retain, for example, the ability ascribed to corresponding full length (or non-fragment) equivalent.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, or at least twenty-nine biomarkers, or at least thirty or more biomarkers) used in the methods and compositions is TNF-R1 (also known as Tumor Necrosis Factor Receptor Superfamily Member 1A, Tumor Necrosis Factor Receptor 1A Isoform Beta, Tumor Necrosis Factor-Alpha Receptor, Tumor Necrosis Factor Binding Protein 1, Tumor Necrosis Factor Receptor Type 1, TNF-RI, TNFR1, TNF-R, CD120a Antigen, CD120a, P55, TNF-R55, P55-R, P60, TNFR60, TBP1, FPF). The sequence of TNF-R1 (numerous isoforms are known) can be found at, for example, UniProt Accession No. P19438.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is TNF-R2 (also known as Tumor Necrosis Factor Receptor Superfamily Member 1B, Tumor Necrosis Factor Receptor Type II, Tumor Necrosis Factor Receptor 2, Tumor Necrosis Factor Binding Protein 2, Tumor Necrosis Factor Beta Receptor, P80 TNF-Alpha Receptor, TNF-RH, TNFR2, TNFR2, TNFBR, TBPII, CD120b Antigen, CD120b, P75, P75 TNF Receptor, TNF-R75, P75TNFR, TNFR80). The sequence of TNF-R2 can be found at, for example, UniProt Accession No. P20333.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight or all at least twenty-nine biomarkers) used in the methods and compositions is CD27 (also known as Tumor Necrosis Factor Receptor Superfamily Member 7, T-Cell Activation Antigen CD27, CD27L Receptor, CD27 Antigen, CD27 Molecule, TNFRSF7, TNF-RSF7, T14, T Cell Activation Antigen S152, LPFS2, S152, Tp55). The sequence of CD27 can be found at, for example, UniProt Accession No. P26842.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight or all at least twenty-nine biomarkers) used in the methods and compositions is LTBR (also known as Lymphotoxin Beta Receptor, Tumor Necrosis Factor Receptor Superfamily Member 3, Tumor Necrosis Factor Receptor 2-Related Protein, Tumor Necrosis Factor Receptor Type III, Tumor Necrosis Factor C Receptor, D125370, TNFRSF3, TNFCR, TNFR3, TNF-R3, Lymphotoxin B Receptor, LT-BETA-R, TNF-R-III, TNFR2-RP, TNF-RIII, TNFR-III, TNFR-RP). The sequence of LTBR can be found at, for example, UniProt Accession No. P36941.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is TNF-RSF6B (also known as TNF Receptor Superfamily Member 6b, Tumor Necrosis Factor Receptor Superfamily, Member 6b, Decoy, Tumor Necrosis Factor Receptor Superfamily Member 6B, TNFRSF6B, Decoy Receptor For Fas Ligand, DCR3, M68, TR6, Decoy Receptor 3 Variant 1, Decoy Receptor 3 Variant 2, Decoy Receptor 3, DJ583P15.1.1, M68E, DcR3). The sequence of TNF-RSF6B can be found at, for example, UniProt Accession No. 095407. In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is FR-alpha (also known as Folate Receptor Alpha, Folate Receptor 1, Ovarian Tumor-Associated Antigen MOv18, Adult Folate-Binding Protein, Folate Receptor 1 (Adult), Folate Receptor, Adult, KB Cells FBP, FOLR, Folate Binding Protein, FBP, FRalpha, FRA, FOLR1). The sequence of FR-alpha can be found at, for example, UniProt Accession No. P15328.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is TNF-RSF10A (also known as TNFRSF10A TNF Receptor Superfamily Member 10a, Tumor Necrosis Factor Receptor Superfamily, Member 10a, TNF-Related Apoptosis-Inducing Ligand Receptor 1, Death Receptor 4, TRAIL Receptor 1, TRAIL-R1, TRAILR1, APO2, DR4, Tumor Necrosis Factor Receptor Superfamily Member 10a Variant 2, Cytotoxic TRAIL Receptor, CD261 Antigen, TRAILR-1, CD261). The sequence of TNF-RSF10A can be found at, for example, UniProt Accession No. 000220.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is TNF-RSF4 (also known as TNF Receptor Superfamily Member 4, TAX Transcriptionally-Activated Glycoprotein 1 Receptor, Tumor Necrosis Factor Receptor Superfamily Member 4, OX40L Receptor, ACT35 Antigen, CD134 Antigen, TXGP1L, Tax-Transcriptionally Activated Glycoprotein 1 Receptor, Tumor Necrosis Factor Receptor Superfamily, Member 4, Lymphoid Activation Antigene ACT35, OX40 Cell Surface Antigen, OX40 Homologue, ATC35 Antigen, OX40 Antigen, ACT35, CD134, IMD16, OX40). The sequence of TNF-RSF4 can be found at, for example, UniProt Accession No. P43489.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is TNF-RSF14 (also known as TNF Receptor Superfamily Member 14, Tumor Necrosis Factor Receptor Superfamily, Member 14 (Herpesvirus Entry Mediator), Tumor Necrosis Factor Receptor Superfamily Member 14, Herpes Virus Entry Mediator A, Tumor Necrosis Factor Receptor-Like Gene2, Tumor Necrosis Factor Receptor-Like 2, TNFRSF14, Herpesvirus Entry Mediator, Herpesvirus Entry Mediator A, HVEA, HveA, HVEM, TR2, CD40-Like Protein, CD270 Antigen, CD270, LIGHTR, ATAR). The sequence of TNF-RSF14 can be found at, for example, UniProt Accession No. Q92956.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is EDA2R (also known as Ectodysplasin A2 Receptor, Tumor Necrosis Factor Receptor Superfamily Member 27, X-Linked Ectodysplasin-A2 Receptor, EDA-A2 Receptor, TNFRSF27, XEDAR, Tumor Necrosis Factor Receptor Superfamily Member XEDAR, Ectodysplasin A2 Isoform Receptor, EDA-A2R, EDAA2R). The sequence of EDA2R can be found at, for example, UniProt Accession No. Q9HAV5.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is RELT (also known as RELT TNF Receptor, Receptor Expressed In Lymphoid Tissues, Tumor Necrosis Factor Receptor Superfamily Member 19L, TNF-RSF19L, RELT Tumor Necrosis Factor Receptor, TNFRSF19L, Tumor Necrosis Factor Receptor Superfamily, Member 19-Like, AI3C, TRLT). The sequence of RELT can be found at, for example, UniProt Accession No. Q969Z4.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is CD160 (also known as CD160 Molecule, CD160 Antigen, Natural Killer Cell Receptor BY55, BY55, Natural Killer Cell Receptor, Immunoglobulin Superfamily Member, CD160 Transmembrane Isoform, CD160-Delta Ig, NK28, NK1). The sequence of CD160 can be found at, for example, UniProt Accession No. 095971.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is IL-1RT1 (also known as Interleukin 1 Receptor Type 1, CD121 Antigen-Like Family Member A, Interleukin-1 Receptor Type 1, Interleukin-1 Receptor Type I, Interleukin-1 Receptor Alpha, IL-1R-Alpha, IL-1RT-1, IL-1R-1, IL1RA, IL1R, P80, Interleukin 1 Receptor Alpha, Type I, Interleukin 1 Receptor, Type I, CD121a Antigen, D2S1473, CD121A, IL1RT1). The sequence of IL-1RT1 can be found at, for example, UniProt Accession No. P14778.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is DLL1 (also known as Delta Like Canonical Notch Ligand 1, Drosophila Delta Homolog 1, Delta-Like Protein 1, H-Delta-1, Epididymis Secretory Sperm Binding Protein, Delta (Drosophila)-Like 1, Delta-Like 1 (Drosophila), DELTA1, Delta1, Delta, DL1). The sequence of DLL1 can be found at, for example, UniProt Accession No. 000548.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is LAYN (also known as Layilin). The sequence of LAYN can be found at, for example, UniProt Accession No. Q6UX15.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is MMP7 (also known as Matrix Metallopeptidase 7, Matrilysin, Matrix Metalloproteinase 7 (Matrilysin, Uterine), Matrix Metalloproteinase-7, Uterine Metalloproteinase, Pump-1 Protease, Matrin, MPSL1, MMP-7, Matrix Metallopeptidase 7 (Matrilysin, Uterine), Uterine Matrilysin, EC 3.4.24.23, EC 3.4.24, PUMP-1, PUMP1). The sequence of MMP7 can be found at, for example, UniProt Accession No. P09237.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is NBL1 (also known as NBL1, DAN Family BMP Antagonist, Neuroblastoma Suppressor Of Tumorigenicity 1, Neuroblastoma Candidate Region, Suppression Of Tumorigenicity 1, Differential Screening-Selected Gene Aberrant In Neuroblastoma, Neuroblastoma 1, DAN Family BMP Antagonist, DAN Domain Family Member 1, DAND1, DAN, Neuroblastoma, Suppression Of Tumorigenicity 1, Zinc Finger Protein DAN, Protein N03, D1S1733E, NO3, NB). The sequence of NBL1 can be found at, for example, UniProt Accession No. P41271.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is WFDC2 (also known as WAP Four-Disulfide Core Domain 2, WAP Four-Disulfide Core Domain Protein 2, Major Epididymis-Specific Protein E4, Putative Protease Inhibitor WAPS, Epididymal Secretory Protein E4, Epididymal Protein 4, WAPS, HE4, Epididymis-Specific, Whey-Acidic Protein Type, Four-Disulfide Core, Epididymis Secretory Sperm Binding Protein, WAP Domain Containing Protein HE4-V4, DJ461P17.6, EDDM4). The sequence of WFDC2 can be found at, for example, UniProt Accession No. Q14508.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is EFNA4 (also known as Ephrin A4, Ephrin-A4, EPH-Related Receptor Tyrosine Kinase Ligand 4, LERK-4, EPLG4, LERK4, Eph-Related Receptor Tyrosine Kinase Ligand 4, Ligand Of Eph-Related Kinase 4, EFL4). The sequence of EFNA4 can be found at, for example, UniProt Accession No. P52798.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is EPHA2 (also known as EPH Receptor A2, Tyrosine-Protein Kinase Receptor ECK, Ephrin Type-A Receptor 2, EC 2.7.10.1, ECK, Epithelial Cell Receptor Protein Tyrosine Kinase, Soluble EPHA2 Variant 1, Epithelial Cell Kinase, EC 2.7.10, CTRCT6, ARCC2, CTPP1, EphA2, CTPA). The sequence of EPHA2 can be found at, for example, UniProt Accession No. P29317.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is GFR-alpha-1 (also known as GDNF Family Receptor Alpha 1, TGF-Beta-Related Neurotrophic Factor Receptor 1, GDNF Family Receptor Alpha-1, GDNFR-Alpha-1, RET Ligand 1, GDNFRA, RETL1, TRNR1, Glial Cell Line-Derived Neurotrophic Factor Receptor Alpha, PI-Linked Cell-Surface Accessory Protein, GPI-Linked Anchor Protein, GDNF Receptor Alpha-1, GDNFR, RET1L). The sequence of GFR-alpha-1 can be found at, for example, UniProt Accession No. P56159.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is KIM1 (also known as Hepatitis A Virus Cellular Receptor 1, Kidney Injury Molecule 1, T-Cell Immunoglobulin Mucin Family Member 1, T-Cell Immunoglobulin Mucin Receptor 1, T-Cell Membrane Protein 1, TIMD-1, KIM-1, TIM-1, TIMD1, TIM1, TIM, T-Cell Immunoglobulin And Mucin Domain-Containing Protein 1, T Cell Immunoglobin Domain And Mucin Domain Protein 1, CD365 Antigen, HAVCR-1, HAVcr-1, CD365, HAVCR). The sequence of KIM1 can be found at, for example, UniProt Accession No. Q96D42.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is PI3 (also known as Peptidase Inhibitor 3, Skin-Derived Antileukoproteinase, Protease Inhibitor 3, Skin-Derived (SKALP), WAP Four-Disulfide Core Domain Protein 14, Peptidase Inhibitor 3, Skin-Derived, Elastase-Specific Inhibitor, Protease Inhibitor WAP3, Trappin-2, Elafin, WFDC14, SKALP, PI-3, WAP3, ESI, WAP Four-Disulfide Core Domain 14, Pre-Elafin, Cementoin). The sequence of PI3 can be found at, for example, UniProt Accession No. P19957.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is TNFRSF11A (also known as TNF Receptor Superfamily Member 11a, Tumor Necrosis Factor Receptor Superfamily Member 11A, Tumor Necrosis Factor Receptor Superfamily, Member 11a, NFKB Activator, Loss Of Heterozygosity, 18, Chromosomal Region 1, Osteoclast Differentiation Factor Receptor, Receptor Activator Of NF-κB, Paget Disease Of Bone 2, ODFR, RANK, Receptor Activator Of Nuclear Factor-Kappa B, CD265 Antigen, LOH18CR1, TRANCER, CD265, OPTB7, OSTS, PDB2, PLO, OFE). The sequence of TNFRSF11A can be found at, for example, UniProt Accession No. Q9Y6Q6.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is CLM1 (also known as CD300 Molecule Like Family Member F, Immune Receptor Expressed On Myeloid Cells 1, Immunoglobulin Superfamily Member 13, CMRF35-Like Molecule 1, NK Inhibitory Receptor, IREM-1, IgSF13, CLM-1, IREM1, NKIR, Immunoglobin Superfamily Member 13, Inhibitory Receptor IREM1, CD300f Antigen, CD300F, IGSF13, LMIR3). The sequence of CLM1 can be found at, for example, UniProt Accession No. Q8TDQ1.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is TNFRSF12A (also known as TNF Receptor Superfamily Member 12A, Fibroblast Growth Factor-Inducible Immediate-Early Response Protein 14, Tumor Necrosis Factor Receptor Superfamily Member 12A, FGF-Inducible 14, Tweak-Receptor, FN14, type I Transmembrane Protein Fn14, CD266 Antigen, TWEAKR, CD266). The sequence of TNFRSF12A can be found at, for example, UniProt Accession No. Q9NP84.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is TRAIL-R2 (also known as TNF Receptor Superfamily Member 10b, Tumor Necrosis Factor Receptor Superfamily, Member 10b, TNF-Related Apoptosis-Inducing Ligand Receptor 2, Death Receptor 5, TRAILR2, KILLER, TRICK2, ZTNFR9, DR5, P53-Regulated DNA Damage-Inducible Cell Death Receptor (Killer), Tumor Necrosis Factor Receptor-Like Protein ZTNFR9, Death Domain Containing Receptor For TRAIL/Apo-2L, Apoptosis Inducing Protein TRICK2A/2B, Apoptosis Inducing Receptor TRAIL-R2, Cytotoxic TRAIL Receptor-2, Fas-Like Protein, TRAIL Receptor 2, CD262 Antigen, KILLER/DR5, TRICK2A, TRICK2B, TRICKB, CD262). The sequence of TRAIL-R2 can be found at, for example, UniProt Accession No. 014763.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is RGMB (also known as Repulsive Guidance Molecule BMP Co-Receptor B, DRG11-Responsive Axonal Guidance And Outgrowth Of Neurite, Repulsive Guidance Molecule Family Member B, RGM Domain Family, Member B, DRAGON, Repulsive Guidance Molecule B). The sequence of RGMB can be found at, for example, UniProt Accession No. Q6NW40.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is DKK4 (also known as Dickkopf WNT Signaling Pathway Inhibitor 4, Dickkopf-Related Protein 4, Dickkopf-4, HDkk-4, Dickkopf (Xenopus Laevis) Homolog 4, Dickkopf Homolog 4 (Xenopus Laevis), DKK-4). The sequence of DKK4 can be found at, for example, UniProt Accession No. Q9UBT3.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is TH-3 (also known as Trefoil Factor 3, Trefoil Factor 3 (Intestinal), Polypeptide P1.B, ITF, TFI, Intestinal Trefoil Factor, HP1.B, HITF, P1B). The sequence of TFF3 can be found at, for example, UniProt Accession No. Q07654.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is CRELD2 (also known as Cysteine Rich With EGF Like Domains 2, Cysteine-Rich With EGF-Like Domain Protein 2, Protein Disulfide Isomerase CRELD2, Cysteine-Rich With EGF-Like Domains 2, EC 5.3.4.1). The sequence of CRELD2 can be found at, for example, UniProt Accession No. Q6UXH1.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is CADM3 (also known as Cell Adhesion Molecule 3, Immunoglobulin Superfamily Member 4B, Synaptic Cell Adhesion Molecule 3, Brain Immunoglobulin Receptor, SynCAM3, IGSF4B, NECL1, TSLL1, Dendritic Cell Nectin-Like Protein 1 Short Isoform, Immunoglobulin Superfamily, Member 4B, Nectin-Like Protein 1, TSLC1-Like Protein 1, Nectin-Like 1, TSLC1-Like 1, SYNCAM3, Ned-1, IgSF4B, NECL-1, BIgR). The sequence of CADM3 can be found at, for example, UniProt Accession No. Q8N126.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is ADAM22 (also known as ADAM Metallopeptidase Domain 22, Metalloproteinase-Like, Disintegrin-Like, And Cysteine-Rich Protein 2, Disintegrin And Metalloproteinase Domain-Containing Protein 22, A Disintegrin And Metalloproteinase Domain 22, Metalloproteinase-Disintegrin ADAM22-3, ADAM 22, MDC2, EIEE61). The sequence of ADAM22 can be found at, for example, UniProt Accession No. Q9P0K1.

In one embodiment of the disclosure, the at least one biomarker (or one biomarker of the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) used in the methods and compositions is CST3 (also known as Cystatin C, Neuroendocrine Basic Polypeptide, Post-Gamma-Globulin, Gamma-Trace, Cystatin-C, Cystatin C (Amyloid Angiopathy And Cerebral Hemorrhage), Epididymis Secretory Protein Li 2, BA218C14.4 (Cystatin C), Cystatin 3, Cystatin-3, HEL-S-2, ARMD11). The sequence of CST3 can be found at, for example, UniProt Accession No. P01034.

Also disclosed herein are arrays (e.g., protein arrays) or compositions comprising any one or more of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22, for performing the methods described herein. Such arrays may include a support or a substrate for attaching any one or more of the biomarker(s) (or fragments thereof) described herein. Such supports and substrates are known in the art and include covalent and noncovalent interactions. For example, diffusion of applied proteins into a porous surface such a hydrogel allows noncovalent binding of unmodified protein within hydrogel structures. Covalent coupling methods provide a stable linkage and may be applied to a range of proteins. Biological capture methods utilizing a tag (e.g., hexahistidine/Ni-NTA or biotin/avidin) on a protein (e.g., a biomarker) and a partner reagent immobilized on the surface of the substrate provide a stable linkage and bind the protein (e.g., a biomarker) specifically and in reproducible orientation.

In one embodiment, the biomarkers described herein are coated or spotted onto the support or substrate such as chemically derivatized glass, or a glass plate coated with a protein binding agent such as, but not limited to, nitrocellulose.

In another embodiment the biomarkers are provided in the form of an array, such as a microarray. Protein microarrays are known in the art and reviewed for example by Hall et al. (2007) Mech Ageing Dev 128:161-167 and Stoevesandt et al (2009) Expert Rev Proteomics 6:145-157, the disclosures of which are incorporated herein by reference. Microarrays may be prepared by immobilizing purified antigens on a substrate such as a treated microscope slide using a contact spotter or a non-contact microarrayer. Microarrays may also be produced through in situ cell-free synthesis directly from corresponding DNA arrays. A microarray may be included in test panels for performing methods disclosed herein. The production of the microarrays is in certain circumstances performed with commercially available printing buffers designed to maintain the three-dimensional shape of the antigens. In one embodiment, the substrate for the microarray is a nitrocellulose-coated glass slide.

The assays are performed by methods known in the art in which the one or more biomarkers are contacted with a biological sample under conditions that allow the formation of an immunocomplex of a biomarker and an antibody, and detecting the immunocomplex. The presence and amount of the immunocomplex may be detected by methods known in the art, including label-based and label-free detection. For example, label-based detection methods include addition of a secondary antibody that is coupled to an indicator reagent comprising a signal generating compound. The secondary antibody may be an anti-human IgG antibody. Indicator reagents include chromogenic agents, catalysts such as enzyme conjugates, fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors and magnetic particles. Examples of enzyme conjugates include alkaline phosphatase, horseradish peroxidase and beta-galactosidase. Methods of label-free detection include surface plasmon resonance, carbon nanotubes and nanowires, and interferometry. Label-based and label-free detection methods are known in the art and disclosed, for example, by Hall et al. (2007) and by Ray et al. (2010) Proteomics 10:731-748. Detection may be accomplished by scanning methods known in the art and appropriate for the label used, and associated analytical software.

Biological Samples

The expression level of one or more biomarkers (i.e., protein biomarkers) may be determined in a biological sample derived from a subject. A sample derived from a subject is one that originates and is obtained from a subject. Such a sample may be further processed after it is obtained from the subject. For example, protein may be isolated from a sample. In one embodiment, the protein isolated from the sample is also a sample derived from a subject. A biological sample useful for determining the level of one or more proteins may be obtained from essentially any source, as protein expression has been reported in cells, tissues, and fluids throughout the body. However, in one aspect of the disclosure, levels of one or more proteins indicative of a subject having renal decline and/or ESRD, or a risk of having renal decline and/or developing ESRD, may be detected in a sample obtained from a subject non-invasively.

In certain embodiments, the biological sample used for determining the level of one or more protein biomarkers is a sample containing circulating protein biomarkers. Extracellular protein biomarkers freely circulate in a wide range of biological material, including bodily fluids, such as fluids from the circulatory system, e.g., a blood sample or a lymph sample, or from another bodily fluid such as cerebrospinal fluid (CSF), urine or saliva. Accordingly, in some embodiments, the biological sample used for determining the level of one or more protein biomarkers is a bodily fluid, for example, blood, fractions thereof, serum, plasma, urine, saliva, tears, sweat, semen, vaginal secretions, lymph, bronchial secretions, CSF, etc. In some embodiments, the sample is a sample that is obtained non-invasively. In one particular preferred embodiment, the sample is a urine sample. In another embodiment, the sample is a plasma sample.

In some embodiments, the biological sample used for determining the level of one or more protein biomarkers, e.g., a sample containing circulating protein biomarkers, may contain cells. In other embodiments, the biological sample may be free or substantially free of cells (e.g., a serum sample). In some embodiments, a sample containing circulating protein biomarkers, is a blood-derived sample. Exemplary blood-derived sample types include, e.g., a blood sample, a plasma sample, a serum sample, etc. In other embodiments, a sample containing circulating protein biomarkers is a lymph sample. Circulating protein biomarkers are also found in urine and saliva, and biological samples derived from these sources are likewise suitable for determining the level of one or more protein biomarkers.

Determining the Level of Biomarkers in a Sample

As described herein, biomarkers indicative of renal decline and/or ESRD and/or biomarkers indicative of an increased risk of renal decline and/or an increased risk of progression to ESRD are discovered. It is thus contemplated that biomarker levels can be assayed from a sample from a subject, such as a test subject (e.g., a subject who is suspected of having renal decline and/or ESRD, or a subject who is at increased risk of having renal decline and/or ESRD) in order to determine whether the test subject has renal decline and/or ESRD, or whether the test subject is at an increased risk of renal decline and/or an increased risk of progression to ESRD. In certain embodiments, biomarkers, e.g., protein biomarkers, were identified by comparing the levels of certain proteins (e.g., circulating proteins) in, for example, samples from subjects who developed renal decline and/or ESRD, or in samples from subjects with diabetes (T1D, T2D) who were at risk for renal decline and rapid progression to ESRD, and compared to levels of certain proteins (e.g., circulating proteins) in, for example, samples from subjects who did not develop renal decline and/or ESRD, or in samples from subjects with diabetes (T1D, T2D) who were determined to have stable renal function (i.e., were non-progressors), or in samples from healthy control subjects, or in samples of a standard control level or reference level. In other embodiments, biomarkers, e.g., protein biomarkers, were identified by comparing the levels of certain proteins (e.g., circulating proteins) in, for example, samples from subjects who developed renal decline and/or ESRD, or in samples from subjects with diabetes (T1D, T2D) who were at risk for renal decline and rapid progression to ESRD, and compared to known baseline concentration of proteins (e.g., circulating proteins or plasma proteins), known or measured, for example, by a proteomics platform (e.g., SOMAscan platform, and/or OLINK platform). A number of differentially present protein biomarkers were identified in this manner, and were determined to be indicative of a subject having renal decline and/or ESRD, at indicative of an increased risk of renal decline and/or progression to ESRD, which include, but are not limited to, TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22.

The protein biomarkers identified herein can be used to determine whether a subject, for example a subject with T1D or T2D, has or is at risk of developing renal decline and/or ESRD, and whose risk of developing renal decline and/or ESRD was previously unknown. This may be accomplished by determining the level of one or more of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, CRELD2, CADM3, and ADAM22, or combinations thereof, in a biological sample derived from the subject. A difference in the level of one or more of these biomarkers as compared to that in a biological sample derived from a normal subject (i.e., a subject known to not have renal decline and/or ESRD; or a normoalbuminuric control level, or a healthy control level, or a standard control level) may be predictive regarding whether the subject has a risk of developing renal decline and/or ESRD.

The level of one or more biomarkers in a biological sample may be determined by any suitable method. Any reliable method for measuring or detecting the level or amount of protein in a sample may be used. Accordingly, practicing the methods disclosed herein may utilize routine techniques in the field of molecular biology. Basic texts disclosing the general methods of use in this disclosure include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

Quantification of Proteins

The present disclosure relates to a method (e.g., in vitro method) of measuring or detecting the amount of certain protein levels found in a cell, tissue, or sample (e.g., a plasma sample) of a subject, as a means to detect the presence, to assess the risk of developing, diagnosing, prognosing, and/or monitoring the progression of and/or monitoring the efficacy of a treatment for renal decline and/or ESRD. Thus, the first steps of practicing the methods of this disclosure (e.g., in vitro methods of using certain identified biomarkers for diagnosis, prognosis, and/or monitoring of renal decline and/or ESRD) are to obtain a cell, tissue or sample (e.g. a urine sample or a plasma sample) from a test subject and extract protein from the sample.

A. Preparation of Samples

In some embodiments, a biological sample (e.g., a cell, a tissue or plasma sample) is obtained from a subject to be tested or monitored for renal decline and/or ESRD as described herein. Biological samples of the same type should be taken from both a test subject (e.g., a subject suspected to have renal decline and/or ESRD and/or a subject at a risk of developing renal decline and/or ESRD) and a control subject (e.g., a subject not suffering from renal decline and/or ESRD; e.g., a sample from a normoalbuminuric control subject, or from a healthy control subject, or of a known/standard control level)). Collection of a biological sample from a subject, such as a test subject, may be performed in accordance with the standard protocol hospitals or clinics generally follow. An appropriate amount of biological sample (e.g., a cell, a tissue or plasma sample) is collected and may be stored according to standard procedures prior to further preparation.

The analysis of certain protein biomarkers, as described herein, found in biological sample of a subject (e.g., test subject) according to the method disclosed herein may be performed in certain embodiments, using, e.g., a cell, a tissue, a urine sample or a plasma sample. The methods for preparing biological samples for protein extraction are well known among those of skill in the art. For example, a cell population or a tissue sample of a subject (e.g., test subject) should be first treated to disrupt cellular membrane so as to release protein contained within the cells.

For the purpose of detecting the presence of certain protein biomarkers disclosed herein or assessing whether a test subject has or is at risk of developing renal decline and/or ESRD, a biological sample may be collected from the subject and the level of certain protein biomarkers disclosed herein may be measured and then compared to the normal level of these same certain protein biomarkers (e.g., compared to the level of the certain protein biomarkers disclosed herein in same type of biological sample in the subject before the onset of renal decline and/or ESRD, and/or compared to the level of the certain protein biomarkers disclosed herein in same type of biological sample from a healthy control subject (e.g., a subject who does not have T1D or T2D), and/or compared to a known control standard of baseline levels of the certain protein biomarkers disclosed herein). If an increase in the level of the certain protein biomarkers disclosed herein is observed when compared to the normal level of the certain protein biomarkers disclosed herein, the test subject is deemed to have renal decline and/or ESRD or have an increased risk of developing renal decline and/or ESRD. For the purpose of monitoring disease progression or assessing therapeutic effectiveness in renal decline and/or ESRD patients, biological sample from a test subject may be taken at different time points, such that the level of the certain protein biomarkers disclosed herein can be measured to provide information indicating the state of disease. For instance, when the level of the certain protein biomarkers disclosed herein from a test subject shows a general trend of decrease or stabilization over time, the test subject is deemed to be improving or stabilizing in the severity of renal decline and/or ESRD or the therapy the patient has been receiving is deemed effective. A lack of decrease or stabilization in the level of the certain protein biomarkers disclosed herein from a test subject or a continuing trend of increase in the level of the certain protein biomarkers disclosed herein from a test subject would indicate a worsening of the condition and ineffectiveness of the therapy given to the patient. Generally, a comparatively higher level of the certain protein biomarkers disclosed herein seen in a test subject indicates that the test subject has renal decline and/or ESRD and/or that the test subject's renal decline and/or ESRD condition is worsening or that renal decline and/or ESRD is progressing.

B. Preparing Samples for Protein Biomarker Detection

Cell, tissue or blood samples (e.g., a plasma sample) from a subject is suitable for the present disclosure and may be obtained using well-known methods and as described herein. In certain embodiments of the disclosure, a plasma sample is a preferred sample type.

C. Determining the Level of Protein Biomarkers

A protein of any particular identity, such as a protein biomarker(s) as disclosed herein, can be detected using a variety of immunological assays. In some embodiments, a sandwich assay can be performed by capturing the protein (also referred to herein as a polypeptide) from a test sample with an antibody (or antibodies) having specific binding affinity for the protein (or polypeptide). The protein can subsequently be detected using, e.g., a labeled antibody having specific binding affinity for the protein biomarker. One common method of detection is the use of autoradiography by using a radiolabeled detection agent (e.g., a radiolabeled anti-biomarker specific antibody) that is labeled with radioisotopes (e.g., ³H, ¹²⁵I, ³⁵S, ¹⁴C, or ³²P, ^{99m}Tc, or the like). The choice of radioactive isotope depends on research preferences due to ease of synthesis, stability, and half-lives of the selected isotopes. Other labels that can be used for labeling of detection agents (e.g., for labeling of anti-biomarker specific antibody) include compounds (e.g., biotin and digoxigenin), which bind to anti-ligands or antibodies labeled with fluorophores, chemiluminescent agents, fluorophores, and enzymes (e.g., HRP). Such immunological assays can be carried out using microfluidic devices such as microarray protein chips. A protein of interest (e.g., a protein biomarker as disclosed herein) can also be detected by gel electrophoresis (such as 2-dimensional gel electrophoresis) and western blot analysis using specific antibodies (e.g., anti-biomarker specific antibodies). In some embodiments, standard ELISA techniques can be used to detect a given protein (e.g., a protein biomarker as disclosed herein), using an appropriate antibody (or antibodies), e.g., an anti-biomarker specific antibody. In other embodiments, standard western blot analysis techniques can be used to detect a given protein (e.g., a protein biomarker as disclosed herein), using the appropriate antibodies. Alternatively, standard immunohistochemical (IHC) techniques can be used to detect a given protein biomarker, using an appropriate antibody (or antibodies), e.g., an anti-biomarker specific antibody. Both monoclonal and polyclonal antibodies (including an antibody fragment with desired binding specificity) can be used for specific detection of the protein biomarker. Such antibodies and their binding fragments with specific binding affinity to a particular protein (e.g., a protein biomarker as disclosed herein) can be generated by known techniques.

In some embodiments, a protein biomarker as disclosed herein can be detected (e.g., can be detected in a detection assay) with an antibody that binds to the protein biomarker, such as an anti-biomarker specific antibody, or an antigen-binding fragment thereof. In certain embodiments, an anti-biomarker specific antibody is used as a detection agent, such as a detection antibody that binds to a protein biomarker as disclosed herein and detects the protein biomarker (e.g., from a biological sample), such as detects the protein biomarker in a detection assay (e.g., in western blot analysis, immunohistochemistry analysis, autoradiography analysis, and/or ELISA). In certain embodiments, an anti-biomarker specific antibody is used as a capture agent that binds to the protein biomarker and detects the protein biomarker (e.g., from a biological sample), such as detects the protein biomarker in a detection assay (e.g., in western blot analysis, immunohistochemistry analysis, autoradiography analysis, and/or ELISA). In some embodiments, an anti-biomarker specific antibody, or an antigen-binding fragment thereof is labeled for ease of detection. In some embodiments, anti-biomarker specific antibody, or an antigen-binding fragment thereof is radiolabeled (e.g., labeled with a radioisotope, such as labeled with ³H, ¹²⁵I, ³⁵S, ¹⁴C, or ³²P, ^{99m}Tc, or the like), enzymatically labelled (e.g., labeled with an enzyme, such as with horseradish peroxidase (HRP)), fluorescent labeled (e.g., labeled with a fluorophore), labeled with a chemiluminescent agent and/or labeled with a compound (e.g., with biotin and digoxigenin).

Other methods may also be employed for measuring or detecting the level of protein biomarker as disclosed herein in practicing the present disclosure. For instance, a variety of methods have been developed based on the mass spectrometry technology to rapidly and accurately quantify target proteins even in a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res 2009 February; 8(2):787-797.

In other embodiments, the expression of a protein biomarker as disclosed herein is evaluated by assessing the protein biomarker as disclosed herein. In some embodiments, an anti-biomarker specific antibody, or fragment thereof, can be used to assess the protein biomarker. Such methods may involve using IHC, western blot analyses, ELISA, immunoprecipitation, autoradiography, or an antibody array. In particular embodiments, the protein biomarker is assessed using IHC. The use of IHC may allow for quantitation and characterization of the protein biomarker. IHC may also allow an immunoreactive score for the sample in which the expression of the protein biomarker is to be determined. The term “immunoreactive score” (IRS) refers to a number that is calculated based on a scale reflecting the percentage of positive cells (on a scale of 1-4, where 0=0%, 1=<10%, 2=10%-50%, 3=50%-80%, and 4=>80%) multiplied by the intensity of staining (on a scale of 1-3, where 1=weak, 2=moderate, and 3=strong). IRS may range from 0-12.

In certain other embodiments, the SOMAscan—Aptamer-based proteomic platform may be used to determine levels of the protein biomarker as disclosed herein. This platform technology is based on the recognition that unique single-stranded sequences of DNA and RNA, referred to as aptamers, are capable of recognizing folded protein epitopes with high affinity and specificity. This property was further advanced with the use of the SOMAscan platform to assay concentrations of proteins (uses one aptamer per protein). This platform features high throughput capabilities (over 1000 proteins in one sample), with reproducibility and sensitivity.

In certain other embodiments, the OLINK-Proximity Extension Assay based proteomic platform may be used to determine levels of the protein biomarker as disclosed herein. The OLINK Proximity Extension Assay is a molecular technique that merges an antibody-based immunoassay with the powerful properties of PCR and quantitative real-time PCR (qPCR), resulting in a multi-plexable and highly specific method (e.g., uses two antibodies per protein) numerous protein biomarkers can be quantified simultaneously using only 1 μL of plasma/serum. These assays were thoroughly validated and grouped as panels designed to focus on specific diseases or biological processes and were optimized for the expected dynamic range of the target protein concentrations in clinical samples.

In some embodiments, the machine-learned, prognostic risk-score assay is KIDNEYINTELX™. To this end, a random forest model can be trained, and performance (e.g., area under the curve (AUC), positive and negative predictive values (PPV/NPV), and net reclassification index (NRI)) can be compared to a clinical model and KDIGO categories for predicting a composite outcome of estimated glomerular filtration rate (eGFR) decline of ≥5 ml/min/year, >40% sustained decline, or kidney failure within 5 years. In some embodiments, an observational cohort study of patients with prevalent diabetic kidney disease (DKD)/banked plasma from two HER-linked biobanks can be used. KIDNEYINTELX™ can provide improved prediction of kidney outcomes over KDIGO (Kidney Disease: Improving Global Outcomes) guidelines and clinical models in individuals with early stages of DKD.

The decliner protein biomarkers described herein can be measured in a proprietary, analytically validated multiplex format using the Mesoscale platform (MesoScale Diagnostics, Gaithersburg, Maryland, USA), which employs electrochemiluminescence detection methods combined with patterned arrays to allow for multiplexing of assays. Each sample can be run in duplicate, along with quality control samples with known low, moderate, and high concentrations of each biomarker on each plate. Assay precision can be assessed using a panel of reference samples that span the measurement range. Levey-Jennings plots can be employed and Westguard rules can be followed the for re-run of samples. The laboratory personnel performing the biomarker assays may be blinded to all clinical information.

### III. Predictive Methods and Compositions Relating to Determining Risk of RD and ESRD

Provided herein are methods, compositions and systems for detecting one or more protein biomarkers (e.g., TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22) in a sample from a subject with, or suspected of having, renal decline and/or ESRD. In certain embodiments, such methods, compositions and systems are used to determine the approximate risk of renal decline (RD) or end-stage renal disease (ESRD) for a subject. In particular embodiments, the compositions and systems are composed of a sample from a subject with, or suspected of having renal decline (RD), and a panel composed of at least two (or at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty or more) RD-associated proteins. In other embodiments, the compositions and systems are composed of a sample from a subject with, or suspected of having end-stage renal disease (ESRD), and a panel composed of at least two (or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, at least twenty-nine biomarkers, or at least thirty or more biomarkers) ESRD-associated proteins.

Provided herein are methods, compositions, and systems (e.g., panels or kits) for determining and/or predicting a subject's risk of having and/or developing renal decline and/or ESRD based on measuring or detecting the levels of biomarkers (e.g., a combination of biomarkers) in one or more of the panels disclosed herein.

In some embodiments, the levels at least two RD-associated proteins are useful for determining the approximate risk of renal decline (RD) in a subject. In some embodiments, the levels at least three RD-associated proteins are useful for determining the approximate risk of renal decline (RD) in a subject. In some embodiments, the levels at least four RD-associated proteins are useful for determining the approximate risk of renal decline (RD) in a subject. In some embodiments, the levels at least five RD-associated proteins are useful for determining the approximate risk of renal decline (RD) in a subject. In some embodiments, the levels at least six RD-associated proteins are useful for determining the approximate risk of renal decline (RD) in a subject. In some embodiments, the levels at least seven RD-associated proteins are useful for determining the approximate risk of renal decline (RD) in a subject. In some embodiments, the levels at least eight RD-associated proteins are useful for determining the approximate risk of renal decline (RD) in a subject. In some embodiments, the levels at least nine RD-associated proteins are useful for determining the approximate risk of renal decline (RD) in a subject. In some embodiments, the levels at least ten RD-associated proteins are useful for determining the approximate risk of renal decline (RD) in a subject. In some embodiments, the levels at least eleven RD-associated proteins are useful for determining the approximate risk of renal decline (RD) in a subject. In some embodiments, the levels at least twelve RD-associated proteins are useful for determining the approximate risk of renal decline (RD) in a subject. In some embodiments, the levels at least thirteen RD-associated proteins are useful for determining the approximate risk of renal decline (RD) in a subject. In some embodiments, the levels at least fourteen RD-associated proteins are useful for determining the approximate risk of renal decline (RD) in a subject. In some embodiments, the levels at least fifteen RD-associated proteins are useful for determining the approximate risk of renal decline (RD) in a subject. In some embodiments, the levels at least sixteen RD-associated proteins are useful for determining the approximate risk of renal decline (RD) in a subject. In some embodiments, the levels at least seventeen RD-associated proteins are useful for determining the approximate risk of renal decline (RD) in a subject. In some embodiments, the levels at least eighteen RD-associated proteins are useful for determining the approximate risk of renal decline (RD) in a subject. In some embodiments, the levels at least nineteen RD-associated proteins are useful for determining the approximate risk of renal decline (RD) in a subject. In some embodiments, the levels at least twenty RD-associated proteins are useful for determining the approximate risk of renal decline (RD) in a subject. In some embodiments, the levels at least twenty-one RD-associated proteins are useful for determining the approximate risk of renal decline (RD) in a subject. In some embodiments, the levels at least twenty-two RD-associated proteins are useful for determining the approximate risk of renal decline (RD) in a subject. In some embodiments, the levels at least twenty-three RD-associated proteins are useful for determining the approximate risk of renal decline (RD) in a subject. In some embodiments, the levels at least twenty-four RD-associated proteins are useful for determining the approximate risk of renal decline (RD) in a subject. In some embodiments, the levels at least twenty-five RD-associated proteins are useful for determining the approximate risk of renal decline (RD) in a subject. In some embodiments, the levels at least twenty-six RD-associated proteins are useful for determining the approximate risk of renal decline (RD) in a subject. In some embodiments, the levels at least twenty-seven RD-associated proteins are useful for determining the approximate risk of renal decline (RD) in a subject. In some embodiments, the levels at least twenty-eight RD-associated proteins are useful for determining the approximate risk of renal decline (RD) in a subject. In some embodiments, the levels at least twenty-nine RD-associated proteins are useful for determining the approximate risk of renal decline (RD) in a subject. In some embodiments, the levels at least thirty or more RD-associated proteins are useful for determining the approximate risk of renal decline (RD) in a subject. Described below are exemplary, non-limiting, methods and systems that are employed using the biomarker panels on samples from subjects with, or suspected of having, renal decline.

In some embodiments, the levels at least two ESRD-associated proteins are useful for determining the approximate risk of end stage renal disease (ESRD) in a subject. In some embodiments, the levels at least three ESRD-associated proteins are useful for determining the approximate risk of end stage renal disease (ESRD) in a subject. In some embodiments, the levels at least four ESRD-associated proteins are useful for determining the approximate risk of end stage renal disease (ESRD) in a subject. In some embodiments, the levels at least five ESRD-associated proteins are useful for determining the approximate risk of end stage renal disease (ESRD) in a subject. In some embodiments, the levels at least six ESRD-associated proteins are useful for determining the approximate risk of end stage renal disease (ESRD) in a subject. In some embodiments, the levels at least seven ESRD-associated proteins are useful for determining the approximate risk of end stage renal disease (ESRD) in a subject. In some embodiments, the levels at least eight ESRD-associated proteins are useful for determining the approximate risk of end stage renal disease (ESRD) in a subject. In some embodiments, the levels at least nine ESRD-associated proteins are useful for determining the approximate risk of end stage renal disease (ESRD) in a subject. In some embodiments, the levels at least ten ESRD-associated proteins are useful for determining the approximate risk of end stage renal disease (ESRD) in a subject. In some embodiments, the levels at least eleven ESRD-associated proteins are useful for determining the approximate risk of end stage renal disease (ESRD) in a subject. In some embodiments, the levels at least twelve ESRD-associated proteins are useful for determining the approximate risk of end stage renal disease (ESRD) in a subject. In some embodiments, the levels at least thirteen ESRD-associated proteins are useful for determining the approximate risk of end stage renal disease (ESRD) in a subject. In some embodiments, the levels at least fourteen ESRD-associated proteins are useful for determining the approximate risk of end stage renal disease (ESRD) in a subject. In some embodiments, the levels at least fifteen ESRD-associated proteins are useful for determining the approximate risk of end stage renal disease (ESRD) in a subject. In some embodiments, the levels at least sixteen ESRD-associated proteins are useful for determining the approximate risk of end stage renal disease (ESRD) in a subject. In some embodiments, the levels at least seventeen ESRD-associated proteins are useful for determining the approximate risk of end stage renal disease (ESRD) in a subject. In some embodiments, the levels at least eighteen ESRD-associated proteins are useful for determining the approximate risk of end stage renal disease (ESRD) in a subject. In some embodiments, the levels at least nineteen ESRD-associated proteins are useful for determining the approximate risk of end stage renal disease (ESRD) in a subject. In some embodiments, the levels at least twenty ESRD-associated proteins are useful for determining the approximate risk of end stage renal disease (ESRD) in a subject. In some embodiments, the levels at least twenty-one ESRD-associated proteins are useful for determining the approximate risk of end stage renal disease (ESRD) in a subject. In some embodiments, the levels at least twenty-two ESRD-associated proteins are useful for determining the approximate risk of end stage renal disease (ESRD) in a subject. In some embodiments, the levels at least twenty-three ESRD-associated proteins are useful for determining the approximate risk of end stage renal disease (ESRD) in a subject. In some embodiments, the levels at least twenty-four ESRD-associated proteins are useful for determining the approximate risk of end stage renal disease (ESRD) in a subject. In some embodiments, the levels at least twenty-five ESRD-associated proteins are useful for determining the approximate risk of end stage renal disease (ESRD) in a subject. In some embodiments, the levels at least twenty-six ESRD-associated proteins are useful for determining the approximate risk of end stage renal disease (ESRD) in a subject. In some embodiments, the levels at least twenty-seven ESRD-associated proteins are useful for determining the approximate risk of end stage renal disease (ESRD) in a subject. In some embodiments, the levels at least twenty-eight ESRD-associated proteins are useful for determining the approximate risk of end stage renal disease (ESRD) in a subject. In some embodiments, the levels at least twenty-nine ESRD-associated proteins are useful for determining the approximate risk of end stage renal disease (ESRD) in a subject. In some embodiments, the levels at least thirty or more ESRD-associated proteins are useful for determining the approximate risk of end stage renal disease (ESRD) in a subject. Described below are exemplary, non-limiting, methods and systems that are employed using the biomarker panels on samples from subjects with, or suspected of having, renal decline.

The levels of the RD-associated proteins are measured and may be used in an algorithm to determine an approximate risk score for RD. In some embodiments, the approximate risk score algorithm includes the serum (or plasma) level for each protein (or nucleic acid) biomarker adjusted by a pre-determined coefficient to generate an adjusted RD-associated protein value for each biomarker, where the adjusted RD-associated protein value for each of the biomarkers measured are added (or multiplied) together to generate the approximate risk score for RD. In other embodiments, the approximate risk score algorithm includes determining an albumin-to-creatinine ratio (ACR) for the subject adjusted by a pre-determined coefficient to generate an adjusted ACR value, where the adjusted ACR value is added (or multiplied) together with the adjusted RD-associated protein value for each of the biomarkers measured to generate the approximate risk score for RD. In another embodiment, the approximate risk score algorithm includes determining a systolic blood pressure (SBP) for the subject adjusted by a pre-determined coefficient to generate an adjusted SBP value, where the adjusted SBP value is added (or multiplied) together with the adjusted RD-associated protein value for each of the biomarkers measured (and optionally with the adjusted ACR value) to generate the approximate risk score for RD. In another embodiment, the approximate risk score algorithm includes determining an estimated glomerular filtration rate (eGFR) for the subject adjusted by a pre-determined coefficient to generate an adjusted eGFR value, where the adjusted eGFR value is added (or multiplied) together with the adjusted RD-associated protein value for each of the biomarkers measured (and optionally with the adjusted ACR value and/or with the adjusted SBP value) to generate the approximate risk score for RD.

The levels of the ESRD-associated proteins are measured and may be used in an algorithm to determine an approximate risk score for ESRD. In some embodiments, the approximate risk score algorithm includes the serum (or plasma) level for each protein (or nucleic acid) biomarker adjusted by a pre-determined coefficient to generate an adjusted ESRD-associated protein value for each biomarker, where the adjusted ESRD-associated protein value for each of the biomarkers measured are added (or multiplied) together to generate the approximate risk score for ESRD. In other embodiments, the approximate risk score algorithm includes determining an albumin-to-creatinine ratio (ACR) for the subject adjusted by a pre-determined coefficient to generate an adjusted ACR value, where the adjusted ACR value is added (or multiplied) together with the adjusted ESRD-associated protein value for each of the biomarkers measured to generate the approximate risk score for ESRD. In another embodiment, the approximate risk score algorithm includes determining a systolic blood pressure (SBP) for the subject adjusted by a pre-determined coefficient to generate an adjusted SBP value, where the adjusted SBP value is added (or multiplied) together with the adjusted ESRD-associated protein value for each of the biomarkers measured (and optionally with the adjusted ACR value) to generate the approximate risk score for ESRD. In another embodiment, the approximate risk score algorithm includes determining an estimated glomerular filtration rate (eGFR) for the subject adjusted by a pre-determined coefficient to generate an adjusted eGFR value, where the adjusted eGFR value is added (or multiplied) together with the adjusted ESRD-associated protein value for each of the biomarkers measured (and optionally with the adjusted ACR value and/or with the adjusted SBP value) to generate the approximate risk score for ESRD.

In some embodiments, the approximate risk score may be used by health care personnel to facilitate deciding on treatment of patients suspected of having renal decline and/or ESRD or for ruling patients out as in need of treatment, monitoring, or inclusion in or exclusion from) a clinical trial.

In certain embodiments, the protein or proteins that are detected as part of a panel described above include at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty-one, or at least twenty-two, or at least twenty-three, or at least twenty-four, or at least twenty-five, or at least twenty-six, or at least twenty-seven, or at least twenty-eight, or at least twenty-nine, or at least thirty or more biomarkers selected from any one of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22.

### VI. Methods of Treatment or Prevention

Methods and compositions for treating or preventing renal decline and/or ESRD in a subject in need thereof are also featured in the disclosure. In one embodiment, the present disclosure provides methods of treating a subject having renal decline and/or ESKD, a subject suspected of having renal decline and/or ESKD, or a subject who is at a risk of developing renal decline and/or ESKD. In certain embodiments, methods of treatment disclosed herein improves kidney function (also referred to herein as “renal function”) in such subjects identified as being at risk of ESRD.

In one embodiment, the present disclosure provides methods of treating a subject with, or suspected of having, renal decline, e.g., a subject having elevated levels of one or more of the RD-associated protein biomarkers selected from the group consisting of TNF-R1, TNF-R2, CD27, LTBR, TNF-RSF6B, TNF-RSF10A, TNF-RSF4, EDA2R, RELT, CD160, IL-1RT1, DLL1, LAYN, MMP7, NBL1, PI3, WFDC2, EFNA4, EPHA2, GFR-alpha-1, and KIM1. In other embodiments, a subject having a disorder associated with chronic kidney disease (e.g., T1D or T2D) may be treated using the methods described herein without having been identified by the predictive methods of the invention. Accordingly, in one embodiment, the disclosure provides a method of treating a subject who has renal decline, or who has been identified as being at risk for developing renal decline, comprising determining the relative level of a RD-associated protein in a sample from the subject, wherein a higher level of the RD-associated protein in the sample relative to a non-decliner control level of the RD-associated protein (or relative to a normoalbuminuric control level of the RD-associated protein, or relative to a healthy control level of the RD-associated protein, or relative to a standard control level of the RD-associated protein) indicates that the subject has or is at risk of developing renal decline, and administering a therapeutically effective amount of the RD-associated protein and/or an RD-associated protein antagonist to the subject, such that renal decline in the subject is treated or prevented.

In another embodiment, the present disclosure provides methods of treating a subject with, or suspected of having, ESRD, e.g., a subject having elevated levels of one or more of the ESRD-associated protein biomarkers selected from the group consisting of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, and KIM1. In other embodiments, a subject having a disorder associated with chronic kidney disease (e.g., T1D or T2D) may be treated using the methods described herein without having been identified by the predictive methods of the invention. Accordingly, in one embodiment, the invention relates to a method of treating a subject who has ESRD, or who has been identified as being at risk for developing ESRD, comprising determining the relative level of a ESRD-associated protein in a sample from the subject, wherein a higher level of the ESRD-associated protein in the sample relative to a non-progressor control level of the ESRD-associated protein (or relative to a normoalbuminuric control level of the ESRD-associated protein, or relative to a healthy control level of the ESRD-associated protein, or relative to a standard control level of the ESRD-associated protein) indicates that the subject has or is at risk of developing ESRD, and administering a therapeutically effective amount of the ESRD-associated protein and/or an ESRD-associated protein antagonist to the subject, such that ESRD in the subject is treated or prevented.

Therapeutic agents useful in the invention may include, but are not limited to, an antagonist of TNF-R1, an antagonist of TNF-R2, an antagonist of CD27, an antagonist of LTBR, an antagonist of TNF-RSF6B, an antagonist of FR-alpha, an antagonist of TNF-RSF10A, an antagonist of TNF-RSF4, an antagonist of TNF-RSF14, an antagonist of EDA2R, an antagonist of RELT, an antagonist of CD160, an antagonist of IL-1RT1, an antagonist of DLL1, an antagonist of LAYN, an antagonist of MMP7, an antagonist of NBL1, an antagonist of PI3, an antagonist of WFDC2, an antagonist of EFNA4, an antagonist of EPHA2, an antagonist of GFR-alpha-1, an antagonist of CST3, an antagonist of KIM1, an antagonist of TNFRSF11A, an antagonist of CLM1, an antagonist of TNFRSF12A, an antagonist of TRAIL-R2, an antagonist of RGMB, and an antagonist of DKK4, an antagonist of TFF3, an antagonist of CRELD2, and an antagonist of CADM3, and an antagonist of ADAM22.

In one embodiment, the subject is a rapid decliner. In another embodiment, the subject is a slow decliner. In one embodiment, the subject is an early renal decliner (RD^{early}). In another embodiment, the subject is a late renal decliner (RD^{late}). In one embodiment, the subject is a rapid progressor. In another embodiment, the subject is a slow progressor. In further embodiments, the subject has diabetes (e.g., T1D or T2D) or high blood pressure.

In other embodiments, the subject is a non-diabetic patient. In some embodiments, the non-diabetic patient has a fasting blood glucose level of less than 100 mg/dL (5.6 mmol/L). In other embodiments, the non-diabetic patient has a random blood glucose level (i.e., blood glucose level tested without a fasting requirement) of less than 140 mg/dL (7.8 mmol/L). In some embodiments, the non-diabetic patient has an HbA1C % (i.e., glycated hemoglobin test or A1C test) of less than 5.7% A1C. In other embodiments, the non-diabetic patient has an oral glucose tolerance level of less than 140 mg/dL (7.8 mmol/L). In preferred embodiments, the blood sample is a plasma sample.

The methods of the invention also include, in certain embodiments, administering an additional agent to the subject, for example an anti-fibrosis agent. Exemplary agents include, but are not limited to angiotensin-converting enzyme inhibitors (ACEI) and angiotensin II receptor type 1 blockers (ARB), renin inhibitors (aliskiren, enalkiren, zalkiren), mineralocorticoid receptor blockers (spironolacton, eplerenone), vasopeptidase inhibitors (e.g. AVE7688, omapatrilat).

Further, nucleic acid molecules (e.g., mRNA and/or miRNA and/or siRNA nucleic acid molecules) useful in the therapeutic methods described herein may be synthetic. The term “synthetic” means the nucleic acid molecule is isolated and not identical in sequence (the entire sequence) and/or chemical structure to a naturally-occurring nucleic acid molecule, such as an endogenous precursor miRNA molecule. While in some embodiments, nucleic acids of the invention do not have an entire sequence that is identical to a sequence of a naturally-occurring nucleic acid, such molecules may encompass all or part of a naturally-occurring sequence. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA. The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together.

It is understood that a “synthetic nucleic acid” of the invention means that the nucleic acid does not have a chemical structure or sequence of a naturally occurring nucleic acid. Consequently, it will be understood that the term “synthetic miRNA” refers to a “synthetic nucleic acid” that functions in a cell or under physiological conditions as a naturally occurring miRNA.

A nucleic acid may be made by any technique known to one of ordinary skill in the art, such as for example, chemical synthesis, enzymatic production or biological production.

Nucleic acid synthesis is performed according to standard methods. See, for example, Itakura and Riggs (1980). Additionally, U.S. Pat. Nos. 4,704,362, 5,221,619, and 5,583,013 each describe various methods of preparing synthetic nucleic acids. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR (see for example, U.S. Pat. Nos. 4,683,202 and 4,682,195, each incorporated herein by reference), or the synthesis of an oligonucleotide described in U.S. Pat. No. 5,645,897, incorporated herein by reference.

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors, plasmids, cosmids, and other vehicles for delivery a nucleic acid to a cell, which may be the target cell or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

In certain embodiments, the nucleic acid molecules of the present invention are not synthetic. In some embodiments, the nucleic acid molecule has a chemical structure of a naturally occurring nucleic acid and a sequence of a naturally occurring nucleic acid, such as the exact and entire sequence of a single stranded primary miRNA (see Lee 2002), a single-stranded precursor miRNA, or a single-stranded mature miRNA. In addition to the use of recombinant technology, such non-synthetic nucleic acids may be generated chemically, such as by employing technology used for creating oligonucleotides.

The present invention involves in some embodiments delivering a nucleic acid into a cell. This may be related to a therapeutic or diagnostic application.

The polynucleotide may be incorporated within a variety of macromolecular assemblies or compositions. Such complexes for delivery may include a variety of liposomes, nanoparticles, and micelles, formulated for delivery to a patient. The complexes may include one or more fusogenic or lipophilic molecules to initiate cellular membrane penetration. Such molecules are described, for example, in U.S. Pat. Nos. 7,404,969 and 7,202,227, which are hereby incorporated by reference in their entireties.

The composition or formulation may employ a plurality of therapeutic polynucleotides, each independently as described herein. For example, the composition or formulation may employ from 1 to 5 miRNA inhibitors and/or miRNA mimetics.

The polynucleotides of the invention may be formulated as a variety of pharmaceutical compositions. Pharmaceutical compositions will be prepared in a form appropriate for the intended application. Generally, this will entail preparing compositions that are essentially free of pyrogens, as well as other impurities that could be harmful to humans or animals. Exemplary delivery/formulation systems include colloidal dispersion systems, macromolecule complexes, nanocapsules, microspheres, beads, and lipid-based systems including oil-in-water emulsions, micelles, mixed micelles, and liposomes. Commercially available fat emulsions that are suitable for delivering the nucleic acids of the invention to cardiac and skeletal muscle tissues include Intralipid, Liposyn, Liposyn II, Liposyn III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. Nos. 5,981,505; 6,217,900; 6,383,512; 5,783,565; 7,202,227; 6,379,965; 6,127,170; 5,837,533; 6,747,014; and WO03/093449, which are hereby incorporated by reference in their entireties.

The pharmaceutical compositions and formulations may employ appropriate salts and buffers to render delivery vehicles stable and allow for uptake by target cells. Aqueous compositions of the present invention comprise an effective amount of the delivery vehicle comprising the inhibitor polynucleotides or miRNA polynucleotide sequences (e.g. liposomes or other complexes), dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” may include one or more solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Supplementary active ingredients also can be incorporated into the compositions.

Administration or delivery of the pharmaceutical compositions according to the present invention may be via any route so long as the target tissue is available via that route. For example, administration may be by intradermal, subcutaneous, intramuscular, intraperitoneal or intravenous injection, or by direct injection into target tissue (e.g., cardiac tissue). Pharmaceutical compositions comprising miRNA inhibitors or expression constructs comprising miRNA sequences may also be administered by catheter systems or systems that isolate coronary circulation for delivering therapeutic agents to the heart. Various catheter systems for delivering therapeutic agents to the heart and coronary vasculature are known in the art. Some non-limiting examples of catheter-based delivery methods or coronary isolation methods suitable for use in the present invention are disclosed in U.S. Pat. Nos. 6,416,510; 6,716,196; 6,953,466, WO 2005/082440, WO 2006/089340, U.S. Patent Publication No. 2007/0203445, U.S. Patent Publication No. 2006/0148742, and U.S. Patent Publication No. 2007/0060907, which are all hereby incorporated by reference in their entireties.

The compositions or formulations may also be administered parenterally or intraperitoneally. By way of illustration, solutions of the conjugates as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use or catheter delivery include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agent(s), for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by their use in the compositions of agents delaying absorption, for example, aluminum monostearate and gelatin.

Sterile injectable solutions may be prepared by incorporating the conjugates in an appropriate amount into a solvent along with any other ingredients (for example as enumerated above) as desired. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The invention is further illustrated by the following examples, which should not be construed as limiting. The entire contents of all references, patents and published patent applications cited throughout this application are hereby incorporated by reference in their entirety.

### EXAMPLES

Described herein are in vitro studies to evaluate biomarkers useful diagnosing, prognosing, and identifying subjects with, or suspected of having, renal decline and/or ESRD (ESKD). The following examples are included for purpose of illustration only and are not intended to be limiting.

### Research Design and Methods

The following research design and methods relate to the study described in Examples 1 to 4.

### Study Subjects

Clinical Characteristics of Diabetic Subjects in the Chronic Renal Insufficiency Cohort (CRIC) Study

The CRIC study is a multicenter prospective study of subjects with chronic kidney disease (CKD) in the U.S. The primary goals of the study are to identify risk factors for progression of CKD and cardiovascular disease among adults aged 21 to 74 years at baseline with mild to moderate CKD. From 2003 to 2008 there were 3,939 ethnically/racially diverse subjects enrolled through 7 clinics into the study, among them 1,908 had diabetes with 80% or more T2D. Participants were followed until the development of ESRD, death, loss to follow-up or Mar. 31, 2013, whichever came first. Participants returned annually for in-person follow-up visits/examinations. The CRIC participants included in this study include all patients with diabetes who had CKD stage 1-3 at baseline, and had at least 2 years follow-up to determine eGFR slope. This group of subjects is referred to hereinafter as the T2D CRIC cohort. Subjects with CKD stage 4 at baseline will be excluded due to different determinants of ESRD progression.

This cohort is estimated to include between 1500 and 1600 subjects. Previous CRIC publications were used to estimate the clinical characteristics of this cohort (8-10). Table 1 shows clinical characteristics of this T2D CRIC cohort according to three ACR categories at baseline: Normoalbuminuria, Microalbuminuria and Macroalbuminuria. Subjects in these categories had similar clinical characteristics; however, the subjects differed with regard to proportion of fast decliners, ESRD events and deaths. Plasma specimens from all subjects from the T2D CRIC cohort are available for this project together with relevant clinical data.

TABLE 1
Characteristics of the T2D CRIC cohort according
to ACR categories at baseline. Dates estimated
using several previous publications (21-23).

| | Normo- | | Micro- | | Macro- | |
|---|---|---|---|---|---|---|
| | Albuminuria | | Albuminuria | | Albuminuria | |
| Characteristics | n = 505 | | n = 475 | | N = 645 | |
| Caucasian (%) | 45 | | 44 | | 38 | |
| At baseline | | | | | | |
| Age (ys) | 61 | | 62 | | 60 | |
| HbA1c | 7.2 | | 7.6 | | 7.9 | |
| Insulin Rx (%) | 55 | | 53 | | 59 | |
| ACR (mg/g) | <30 | | 30-299 | | >300 | |
| eGFRcr (ml/min) | 51 | | 52 | | 45 | |
| ACE&ARB (%) | 80 | | 79 | | 78 | |
| 5-10 ys of Follow-up | | | | | | |
| # of serum creatine | 7 | (3, 11) | 6 | (3, 11) | 5 | (3, 11) |
| eGFR slope (ml/min/y) | −1.0 | | −1.4 | | −4.1 | |
| Loss eGFR ≥5 | 35 | (7%) | 76 | (19%) | 284 | (44%) |
| ml/min/y (n/%) | | | | | | |
| Cases of ESRD (n/%) | 16 | (3%) | 78 | (16%) | 256 | (39%) |
| Deaths unrelated to | 52 | (10%) | 79 | (17%) | 160 | (25%) |
| ESRD | | | | | | |

Serum creatine-based eGFR slope estimated with ordinary least squares assuming linear regression. Estimated means or proportions are shown.

### Clinical Characteristics of the JKS T2D Cohort

The Joslin Clinic is a major center for treatment of subjects with diabetes. A large proportion of subjects come to the clinic within the first 5 years after diabetes diagnosis and remain under clinic care for a long period of time. Participants in the Joslin Kidney Study (JKS) were recruited from subjects attending Joslin Clinic between 2003 and 2009. Residents of New England with T2D diagnosed after age 30 and age 30-64 at study enrollment were eligible for the study. Subjects were ineligible if they were on dialysis, had renal transplant or had CKD stage 4.

Between 2003 and 2009, 1440 subjects were enrolled, 710 with normoalbuminuria, 471 with albuminuria and CKD stage 1-2 and 255 with albuminuria and CKD stage 3. The subjects were examined during routine visits to the Clinic as baseline examination and biannually afterwards with specimens of blood and urine taken for laboratory determinations and thereafter held in storage at −80° C. Patients with less frequent clinic visits or those who stopped attending the clinic were examined at their homes. All patients were queried against rosters of the United States Renal Data System (USRDS) and the National Death Index (NDI) as of the end of 2015.

Table 2 shows clinical characteristics of the JKS T2D Cohort. In comparison the T2D CRIC cohort (see above), the JKS T2D cohort (see above) had more Caucasians, the cohort was younger, the majority of subjects had normal renal function and lower ACR. Using available determinations of serum creatinine, eGFR slopes were estimated in the whole cohort except for 80 subjects who died or were lost during the first 2 years of follow-up. In total there are 283 fast decliners, and 120 cases of ESRD. The proportion of fast decliners in ESRD cases was the highest among subjects with albuminuria and CKD stage 3. In this cohort there were only 74 deaths unrelated to ESRD. By updating the status of this cohort as of 2018, estimates of eGFR in 700 subjects will be improved and 30 additional ESRD cases and 20 deaths will be identified.

TABLE 2
Characteristics of the Joslin Kidney Study Cohort with
T2D subjects according to ascertainment criteria.

| | Normo- | | Albuminuria | | Albuminuria | |
|---|---|---|---|---|---|---|
| | Albuminuria | | CKD 1 and 2 | | CKD 3 | |
| Characteristics | n = 710 | | n = 475 | | N = 255 | |
| Caucasian (%) | 79% | | 75% | | 76% | |
| At baseline | | | | | | |
| Age (ys) | 57 | (51; 62) | 57 | (50; 61) | 60 | (56; 64) |
| Duration of DM (ys) | 11 | (7; 15) | 10 | (5; 15) | 16 | (11; 22) |
| Insulin Rx (%) | 60% | | 53% | | 60% | |
| ACR (mg/g) | 7 | (5; 11) | 53 | (26; 159) | 195 | (49; 1089) |
| eGFRcr (ml/min) | 95 | (82; 105) | 97 | (82; 106) | 47 | (39; 54) |
| ACE&ARB (%) | 56% | | 79% | | 82% | |
| 6-12 ys of Follow-up | | | | | | |
| # of serum creatine | 12 | (6; 15) | 14 | (8; 19) | 19 | (15; 26) |
| eGFR slope (ml/min/y)* | −1.6 | (−2.4; −0.3) | −2.8 | (−5.4; −0.9) | −4.1 | (−6.3; −0.7) |
| Loss eGFR ≥5 | 43 | (6%) | 107 | (23%) | 133 | (52%) |
| ml/min/y (n/%) | | | | | | |
| Cases of ESRD (n/%) | 14 | (2%) | 24 | (5%) | 82 | (32%) |
| Deaths unrelated to | 22 | (3.1%) | 29 | (6.0%) | 23 | (11.3%) |
| ESRD | | | | | | |

*Serum creatine-based eGFR slope estimated with ordinary least squires assuming linear regression. Data for continuous variables are medians (25^{th}, 75^{th} percentiles.)

In summary, although the compositions of the JKS T2D cohort and the T2D CRIC cohort are different, it is desirable to have such differences to validate the diagnostic and prognostic algorithms.

### SOMAscan—Aptamer-Based Proteomic Platform

Unique single-stranded sequences of DNA and RNA, referred to as aptamers, are capable of recognizing folded protein epitopes with high affinity and specificity. This property was further advanced with the use of the Slow Off-rate Modified Aptamer (SOMAmer) utilized on the SOMAscan platform to assay concentrations of proteins (uses only one aptamer per protein). This platform features high throughput capabilities (over 1000 proteins in one sample), with reproducibility and sensitivity.

### OLINK—Proximity Extension Assay Based Proteomic Platform

The OLINK Proximity Extension Assay is a molecular technique that successfully merges an antibody-based immunoassay with the powerful properties of PCR and quantitative real-time PCR (qPCR), resulting in a multi-plexable and highly specific method (e.g., uses two antibodies per protein) where up to 92 protein biomarkers can be quantified simultaneously using only 1 μL of plasma/serum (11,12). These assays were thoroughly validated and grouped as panels designed to focus on specific diseases or biological processes and were optimized for the expected dynamic range of the target protein concentrations in clinical samples. Eleven panels exist which can measure concentration of 1000 proteins.

### Statistical Analysis

In the analysis, two outcomes are considered.

I. Renal Decline with Two Levels

An eGFR slope of between −80 ml/min/year and −5 ml/min/year (i.e., a fast decliner) and an eGFR slope >=−5 ml/min/year (i.e., a slow decliner), where serum creatinine-based eGFR slope for a given subject is estimated using ordinary least squares assuming linear regression with at least 3 serum creatinine values available and measured at least 1 year apart.

II. Time to Onset of ESRD

The endpoint for this outcome is defined as a composite of three events, 1) initiation of dialysis, 2) renal transplant or 3) death due to major renal cause. Time to the event, i.e. time to ESRD, is defined as time from entering the study to the first event (one of the events listed above) or censoring (e.g. deaths unrelated to ESRD).

Initial Data Analysis:

In the initial analysis of data from the CRIC (described herein) and the JKS studies (described herein), measures of central tendency (means, medians) and variability (standard deviations, ranges) for continuous variables will be measured. Frequency distributions will be provided for categorical data. This preliminary analysis step provides insight into data, distributions of the variables considered, and allows for the determination of additional invalid values not detected earlier during data validation. Logistic regression and Cox regression models will be used, respectively, to evaluate associations of clinical characteristics and serum proteins from the Kidney Biomarker Panel 1 with two outcomes: 1) renal decline (fast versus slow), and 2) time to onset of ESRD in CRIC and in JKS studies.

Primary Data Analysis:

First, the CRIC study data will be used as a training dataset to develop models predictive of probabilities of two qualitative outcomes, specifically 1) binary outcome (fast/slow decliner) with a “success” defined as eGFR slope <−5 ml/min/year, and 2) progression to ESRD within 5 years of follow-up. Thereafter, to externally validate the predictive model developed using the CRIC study data, the JKS data will be used. Finally, the aforementioned predictive models will be used to specify algorithm/decision rule(s) for both outcomes. The process of developing predictive models using the CRIC study data and JKS data, followed by formulating the decision rule(s) consists of several steps: (Step S1) defining a qualitative outcome with risk categories of interest, (Step S2) selecting a class of models predictive of probability of the risk category, (Step S3) model selection, (Step S4) assessing model performance, (Step S5) validating the model, (Step S6) specifying decision rule(s) by selecting cut-off point for the probability of the risk category. These steps are outlined in detail below.

Both qualitative outcomes/endpoints with risk categories of interest (Step S1) were defined above. Classes of predictive models considered in Step S2 are specified separately for each outcome, as provided below.

Logistic Regression Model for the Fast Decliner Outcome Defined as eGFR Slope <−5 Ml/Min/Year

To develop a diagnostic model predictive of probability of being fast decliner, logistic regression models (Step S2) of the form will be fitted:

$$log ( \frac{{p}_{i}}{1 - {p}_{i}} ) = {β}_{0} + {x}_{i}^{′} β , \qquad M1$$

where pᵢ is a conditional probability of eGFR slope being lower than −5 ml/min/year, i.e. being fast decliner, given that subject i is described by a vector of predictors xᵢ obtained during selection of an optimal model. To increase flexibility of model M1, vector xᵢ may also include clinical characteristics by circulating proteins interaction terms. For the full list of predictors, including all circulating proteins, see Tables 2 and 3. The β₀ and β are an intercept and a vector of the fixed effects associated with predictors, xᵢ, respectively. An estimate of pᵢ is a natural choice for a diagnostic score for becoming a fast decliner. An estimate of pᵢ can be obtained from the model M1 by applying logistic transformation

$$\frac{exp ( {η}_{i} )}{1 + exp ( {η}_{i} )}$$

to a linear predictor, nᵢ defined as nᵢ=β₀+xᵢ′β. Binary outcome for model M1, will be created by categorization of the subject-specific eGFR slopes obtained from the individual linear regression model fitted to eGFR values. As an alternative to logistic regression, other more flexible models, such as generalized additive models (GAM), generalized nonlinear regression models (GNLM), and multivariate adaptive regression splines (MARS) that can be seen as a generalization of a logistic model M1 may be implemented.

Cox Regression Model to Predict Probability of the Progression to ESRD within 5 Years of Follow-Up

To develop a prognostic model to predict the probability of progression to ESRD within 5 years of follow-up, Cox regression models (Step S2) will be fitted for time to progression to ESRD of the following form:

h(t|xᵢ)=h₀(t)exp(β₀+xᵢ′β), M2:

where h(t|xᵢ) is a hazard of developing ESRD at time t, given that subject t is described by a vector of predictors xᵢ obtained during selection of an optimal model. It should be noted that vector xᵢ may also include clinical characteristics by circulating proteins interaction terms. For the full list of predictors, including circulating proteins, see Tables 2 and 3. The h₀(t) is a baseline hazard. The β₀ and β are an intercept and a vector of the fixed effects associated with predictors, xᵢ, respectively. To estimate probability, pᵢ, of progression to ESRD during 5 years of follow up from Cox model, we use the corrected group prognosis method advocated by Ghali et al, 2001 (13) will be used. In model M2, time to ESRD event is right censored by death due to non-renal causes and by discontinuation from the study. In an alternate approach, non-renal deaths will be considered together with ESRD event as part of a composite endpoint. This will allow for the assessment of how the developed predictive model is sensitive to changes in the definition of the endpoint of interest.

Model Selection (Step S3)

To select optimal statistical models of the form M1 and M2, several approaches will be used, including forward stepwise selection and a hybrid approach involving both forward and backward selection. In the context of data with a relatively modest number of events relative to the number of predictors, including circulating proteins and their interactions with circulating proteins' levels, such approaches may lead to over-fitting. For this reason, a model subset selection technique will be employed based on shrinkage (a.k.a., regularization). The key feature of such techniques is that the objective function (for example, likelihood function for model M1 and partial likelihood for M2) is regularized by adding a penalty function to it. Optimization of such penalized objective function causes the estimated regression coefficients to be shrunken towards (or be entirely reduced to) zero. It is noted that a priori knowledge pertaining to the candidate circulating proteins or clinical characteristics can be used in these techniques by applying smaller penalties to the corresponding regression coefficients, which subsequently result in smaller degrees of shrinkage. Methods based on shrinkage are especially attractive for model selection in the context of data disclosed herein because such methods alleviate the problem of over-fitting of the model, which in turn typically improves the model's predictive performance. There are two primary regularization techniques: 1) ridge regression (with penalty based on L2 norm), and 2) LASSO (least absolute shrinkage and selection operator; with penalty based on L1 norm applied to beta coefficients). A desired feature of LASSO in the model selection process is that it allows shrinking of the estimates all the way to zero. Unfortunately, in cases where there is a group of highly correlated predictors, LASSO tends to select one predictor and ignore the others in the group. For this reason, an elastic net algorithm, which constructs penalty functions based on both L1 and L2 norms and overcomes the limitations of the LASSO algorithm, may be used. A focal part of these algorithms is the selection of regularization/tuning parameters to achieve good prediction accuracy. To complete this task, a cross-validation technique will be employed. Model selection will be performed using glmnet (Friedman et al, (14) and coxnet (Simon et al, (15) R packages for selecting models M1 and M2, respectively.

Assessment of Prediction Models (Step S4)

To assess performance of prediction models, i.e. logistic regression (M1) and Cox regression (M2), obtained by using procedures described in the Model Selection subsection (Step S3), several metrics will be considered. Predictive performance of the models will be assessed overall, followed by assessing of model calibration and discrimination. Overall performance will be assessed using Brier's score for the logistic model M1. To account for censoring in Cox regression model (model M2), Graf et al (16) will be used as well as use weights created based on probability of censoring to calculate Brier's score. A required assumption is that the censoring mechanism is independent of survival and the subject's history. Calibration of the model measures how accurately model's predictions match overall observed event rates. For logistic regression model, the observed outcome will be plotted by decile of predictions, which effectively is an illustration of the Hosmer-Lemeshow goodness-of-fit test. For the Cox regression model the distribution of Cox-Snell residuals will be investigated in a plot of cumulative hazard vs. residuals.

The discriminative ability of the logistic model, i.e. the ability of the model to separate individuals who develop fast renal decline from those who do not, will be assessed using the c statistic, reclassification tables, net reclassification improvement (NRI), and integrated discrimination improvement (IDI). It should be noted that some alternative definitions of c-statistics for survival model have been proposed, which lead to time-dependent performance curves (17,18).

Validation of Prediction Models (Step S5)

Logistic model (M1) and Cox regression model (M2) developed in Step S3 will be validated employing 10-fold cross-validation technique using CRIC study data. To assess generalizability of models M1 and M2, the JKS data will be used for validation.

Developing Decision Rule for Classifying Subjects into Fast/Slow Decliners (Step S6)

The process of developing decision rule for classifying into fast/slow decliners is similar for the logistic regression (M1) and Cox regression (M2) models. For example, for the logistic regression model, a threshold (cutoff-point), c₀, is established such that 0<c₂<1, for pᵢ, where pᵢ is a conditional probability of eGFR slope being lower than −5 ml/min/year, i.e. being fast decliner. For a given threshold, the classification rule states that, if the estimate of pᵢ, for a given subject is greater than c₀, then the subject i, is classified as a fast decliner, otherwise the subject is classified as a slow decliner. The choice of c₀=0.5, implies that both type of errors, namely false-positive and false-negative classifications are considered to be equally important. Given that the false-negative errors are considered to be more important, and based on expert opinion, a cut-off value of c₀ at 0.3 is assumed. To more formally select optimal cut-off value, the decision curve analysis proposed by Vickers (19) will be used. To assess the clinical usefulness of the proposed classification rule, sensitivity, specificity, accuracy and net benefit will be calculated.

Power and Precision Estimation

To estimate precision of estimates obtained through the proposed decision rule and assess the adequacy of available sample size, a simulation-based power analysis estimating the upper and lower half-sizes of a 95% confidence interval about an odds ratio estimate from the logistic model (M1) was performed. For calculations, lognormal distribution of a putative diagnostic score and underlying effect size (odds ratio) to be uniformly distributed per any one quartile of the score was assumed. As indicated in Table 3, the confidence interval half-sizes with >80% probability to be as narrow or narrower, depending on the sample size and the magnitude of the odds ratio. Further, as indicated in Table 3, the available number of patients is sufficient to attain the precision of the odds ratio estimates approximately 5% or less of the effect size, sufficient for good quality event prognostics.

TABLE 3
Confidence interval precision according
to sample size and odds ratio magnitude.

| Sample size | Odds ratio | Lower CI half-size | Upper CI half-size |
|---|---|---|---|
| 600 | 1.5 | 0.090 | 0.098 |
| 600 | 2 | 0.098 | 0.106 |
| 600 | 2.5 | 0.111 | 0.123 |
| 600 | 3 | 0.128 | 0.142 |
| 600 | 4 | 0.170 | 0.191 |
| 1200 | 1.5 | 0.066 | 0.070 |
| 1200 | 2 | 0.073 | 0.078 |
| 1200 | 2.5 | 0.083 | 0.089 |
| 1200 | 3 | 0.096 | 0.103 |
| 1200 | 4 | 0.128 | 0.139 |

### Example 1: Progressive Renal Decline Leads to ESRD

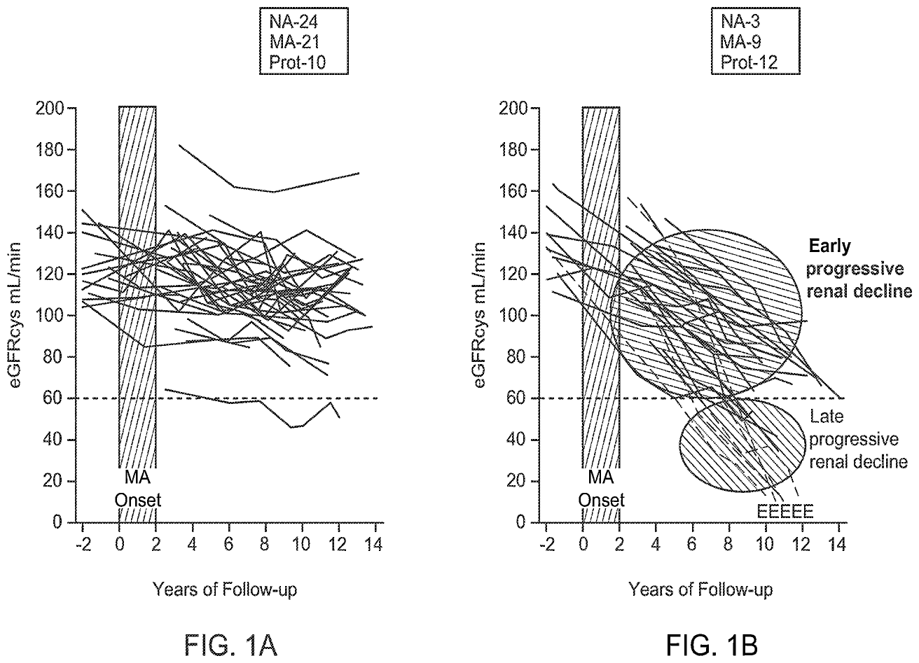

To generate empirical data about trajectories of the decline of renal function in subjects who developed ESRD, the Joslin T1D ESRD Cohort was assembled. This cohort comprises 364 residents of Eastern Massachusetts with T1D (85% Caucasian) who were under the care of the Joslin Clinic in the 1980's and 1990's and who developed ESRD between 1991 and 2011. From the Joslin Clinical Laboratory archives, numerous years of serum creatinine determinations (on average 9±6 years) before onset of ESRD were obtained. These determinations were used to trace eGFR trajectories preceding onset of ESRD (FIG. 1). Most of the first available determinations were in CKD stages 1-2. FIG. 1A shows subjects (n=65) without renal decline (i.e., horizontal eGFR trajectories in non-decliners). FIG. 1B shows subjects (n=24) with progressive renal decline (i.e., eGFR trajectories in decliners). Among decliners, a significant number of them had very fast decline that resulted in progression from normal eGFR to ESRD within 12 years of follow-up. Note that subjects with NA or MA at the last exam had renal decline.

While most eGFR trajectories appeared linear by inspection, validation was performed statistically by fitting both linear and spline models to each patient's data. Using this approach, linear renal decline was observed in the vast majority of patients (i.e. 87%). The remaining 13% of trajectories were sufficiently non-linear to have clinically consequential impacts on anticipated time to ESRD; the trajectories of 6% of the patients accelerated and 7% decelerated.

In summary, a linear inexorable progression to ESRD was the predominate pattern of eGFR decline in this large cohort of T1D. The findings are in agreement with findings in FIG. 1B.

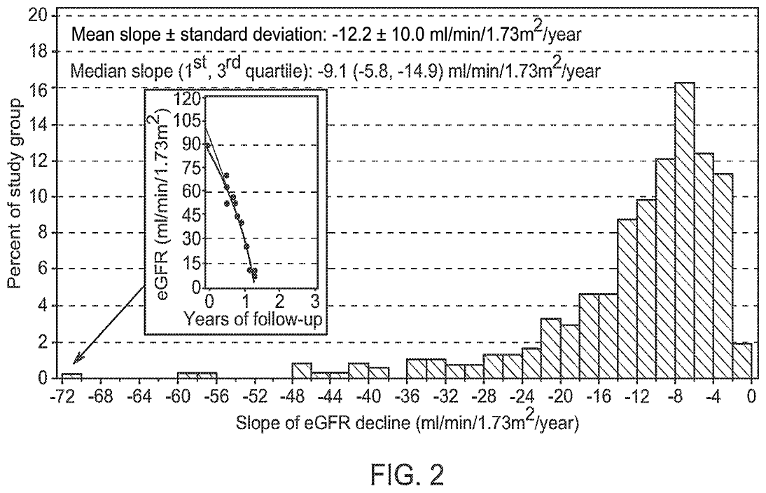

To characterize the distribution of rates of eGFR decline in the Joslin Cohort, the linear component of each trajectory was extracted as a simple slope. Analysis of the slopes indicated that the distribution of the eGFR slopes was highly skewed with the tail to the left—toward steeply negative slopes (FIG. 2). Almost 80% of the cohort had eGFR slope <−5 ml/min/year. The KIDGO guidelines classify this as rapid progression (20). For purposes of this disclosure, an eGFR slope of between −80 ml/min/year and −5 ml/min/year is considered fast renal decline. An alternative method of representing slopes is to express them as the estimated time needed to reach ESRD from a starting point such as an eGFR of 100 ml/min (Table 4). The ranges in the four categories of slopes, when translated into ranges of time to ESRD, would yield: 2-6 yrs, 6-10 yrs, 10-20 yrs and 20-45 yrs. Fast decliners would develop ESRD in 2 to 20 years.

TABLE 4

| | Categories of progressive renal decline | | | |
|---|---|---|---|---|
| | very fast | fast | moderate | slow |
| eGFR slope | <−15 | <−10 | <−5 | ≥−5 |
| (ml/min/1.73 m²/year) | | | | |
| Time to onset of ESRD | 2-6 | 6-10 | 10-20 | 20-45 |
| in years | | | | |

In summary, in T1D, renal function declined over time. In the majority of decliners, renal decline was progressive and linear but variable among subjects. Subjects with normal renal function and with eGFR slope <−5 ml/min/year had very high risk of developing ESRD within 20 years. This interval varied according to baseline eGFR, eGFR slope and competing mortality. Similar findings were found in subjects with T2D (data not shown).

### Example 2: Multi-Cohort Approach to Identify Candidate Biomarkers

Several small cohorts of subjects with well-defined kidney outcomes were used to identify candidate biomarkers. The clinical characteristics of the cohorts are provided in Table 5.

TABLE 5
Clinical characteristics of T1D Joslin patients included in T1D
Joslin Discovery and Replication Cohorts and Validation PIMA
Cohort with T2D. Values are shown as Medians and 1st & 3rd Qs.

| | Discovery Cohort | | Replication Cohort | | Validation Cohort | |
|---|---|---|---|---|---|---|
| | with proteinuria | | with microalbuminuria | | Of T2D PIMA | |
| | (N = 203) | | (N = 148) | | (N = 80) | |
| At baseline exam | | | | | | |
| Ages (years) | 33 | (25, 40) | 35 | (27, 43) | 45 | (40, 52) |
| Duration of DM (years) | 18 | (11, 27) | 20 | (13, 30) | 16 | (11, 20) |
| HbA_{1c} (DCCT, %) | 9.1 | (8.2, 10.5) | 9.6 | (9.1, 11.9) | 10.0 | (8.6, 11.5) |
| Albumin in urine (mg/g) | 753 | (370, 1373) | 54 | (22, 126) | 12 | (3, 118) |
| eGFR (ml/min/1.73 m²) | 100 | (73, 114) | 113 | (99, 123) | 150 | (115, 190) |
| During 7-15 ys follow-up | | | | | | |
| Decliners <−5 ml/min/year | 116 | (57%) | 57 | (39%) | 51 | (64%) |

| | | | | | |
|---|---|---|---|---|---|
| Cases of ESRDs n (%) | 92 | (45%) | NA | 42 | (53%) |

The relatively large number of proteins assayed the OLINK platform and the relatively small number of subjects in each cohort results in statistical methods that may be considered conservative/inefficient for identifying accurate multi-marker diagnostic or prognostic algorithms using each cohort individually. Accordingly, a multi-cohort approach allowed for the identification of a number of candidate proteins that were replicated in each cohort for use in developing a small custom-made proteomics panel. Subsequently, this small proteomics panel will be used in large independent studies for the development of accurate diagnostic and prognostic algorithms. The cohorts described in this example were used in Example 3.

### Example 3: OLINK—Proximity Extension Assay Based Proteomic Platform Analysis

Plasma specimens obtained at baseline from subjects included in the Joslin T1D Discovery Cohort (n=203) described in Example 2 were examined using the OLINK proteomics platform to determine concentration of 1000 circulating proteins contained on all 11 OLINK panels. As a result of this examination 78 proteins strongly (p<10⁻⁴) associated with risk of fast renal decline (an eGFR slope of between −80 ml/min/year and −5 ml/min/year) were identified using logistic analyses. The same proteins were associated with fast progression to ESRD using Cox regression analysis. The proteins associated with progression to ESRD were present on 5 OLINK panels containing 460 proteins. Only these 5 panels were used in the Replication and Validation Cohorts.

In analyzing the data from all 3 cohorts (see Table 4; Example 2), surprisingly of the 78 proteins strongly associated with fast renal decline in the Discovery Cohort, 52 of these proteins were also associated with fast renal decline in the Replication cohort. These 52 proteins were identified as candidate biomarkers for further study. Forty-four of these candidate proteins were also validated in the PIMA T2D cohort (i.e., the PIMA Indian cohort (with normal renal function)). Further analysis of the 52 proteins is described in Example 4.

In conclusion, plasma protein profiles associated with risk of fast renal decline and fast progression to ESRD were strikingly similar among the three cohorts despite significant differences in ACR (medians were 753, 54, 12), GFR (medians 100, 113, 150) and types of diabetes.

### Example 4: Development of the Kidney Biomarker Panel 1

To further validate the 52 candidate proteins identified in the previous example in large cohorts, this set of candidate proteins was reduced to the 21 most informative to build the custom-made OLINK-based biomarker panel. To select these 21 proteins, several approaches were used. First, several cluster analyses were performed to group the 52 candidate proteins into 12 clusters. The highly inter-correlated proteins in each cluster were ranked further by statistical procedures (backward elimination, forward selection) to identify a representative of each cluster with highest association to risk of ESRD. Once the 12 cluster representatives were selected, this set of proteins was enriched up to 21 by additional candidate proteins representing the most promising (least redundant) proteins to be used in developing multi-marker diagnostic and prognostic algorithms A list of proteins included in the Kidney Biomarker Panel 1 is shown in Table 6 below. Table 6 also shows the significance of the association between baseline concentration of the selected proteins and risk of fast renal decline in each of the three study cohorts.

TABLE 6
List of informative proteins of the Kidney Biomarker Panel 1

| | Joslin Proteinuria | Joslin Microalb | Pima |
|---|---|---|---|
| | Cohort | Cohort | Cohort |
| Protein Name | −log₁₀ P | −log₁₀ P | −log₁₀ P |
| TNF-RSF1A | *5.40 | 2.42 | 5.48 |
| TNF-RSF1B | *5.32 | 3.04 | 6.25 |
| TNF-RSF3 | 7.25 | 1.68 | 5.18 |
| TNF-RSF6B | 7.11 | 2.07 | n.s. |
| TNF-RSF7 | 7.16 | 2.85 | 6.37 |
| TNF-RSF10A | 6.73 | 2.33 | 2.92 |
| TNF-RSF4 | 5.17 | 2.07 | 7.17 |
| TNF-RSF19L | *6.91 | 1.52 | 4.54 |
| TNF-RSF27 | *7.45 | 1.39 | 4.12 |
| CD160 | 5.90 | 2.26 | 7.01 |
| IL-1RT1 | *6.62 | 3.26 | n.s. |
| DLL1 | *10.08 | 3.54 | 7.29 |
| LAYN | *8.39 | 3.54 | 8.59 |
| PI3 | 8.27 | 3.16 | 2.88 |
| NBL1 | *4.25 | 1.54 | 1.82 |
| WFDC2 | 10.11 | 3.54 | 5.76 |
| EFNA4 | *6.62 | 1.89 | 6.70 |
| EPHA2 | *9.33 | 2.07 | 6.19 |
| GFR-alpha-1 | 6.01 | 2.85 | 5.29 |
| KIM1 | 13.53 | 6.41 | 7.77 |

The outcome of interest is fast renal decline defined as an eGFR slope of between −80 ml/min/year and −5 ml/min/year. Univariate logistic regression was used to compute p values.
*Proteins associated with progression to ESRD in subjects with CKD stage 3 (SOMAscan results).

Interestingly, the proteins identified in Table 6 may be generally classified into the following protein groups: inflammatory proteins (i.e., TNF-RSF1A, TNF-RSF1B, TNF-RSF3, TNF-RSF6B, TNF-RSF7, TNF-RSF10A, TNF-RSF4, TNF-RSF19L, TNF-RSF27, CD160, and IL-1RT1), pro-fibrotic proteins (i.e., DLL1, LAYN, PI3, NBL1, WFDC2), axon guidance pathway (AGP) proteins (i.e., EFNA4, EPHA2, GFR-alpha-1) and KIM1, which represent biological pathways involved in the etiology of the development of ESRD and the most promising (least redundant) proteins to be used in building the multi-marker diagnostic and prognostic algorithms

The data in Table 7 shows backward elimination (B, p-for-stay <0.1) and forward (F, p-for-entry <0.1) selection of informative proteins to develop diagnostic algorithm to identify T1D subjects with fast renal decline in the Discovery and Replication Cohorts combined. Using Model 1, 11 informative proteins were selected for diagnostic model from among all 21 proteins examined Using Model 2, 11 informative proteins were selected, however, differed slightly from Model 1, when baseline eGFR was considered. Using Model 3, 8 informative proteins were selected, similar as in Model 2, when baseline eGFR and ACR was included. Based on the foregoing, 8-11 proteins may contribute information for the development of a multi-marker diagnostic algorithm to identify subjects with fast renal decline among T1D subjects. It is expected that a similar set of proteins, possibly not identical to the aforementioned set of proteins, may provide information for the development of a prognostic algorithm to estimate the probability of developing ESRD during 5 years of observation.

TABLE 7
Proteins selected by backward elimination (B, p- for-stay <0.1)
and forward (F, p- for-entry <0.1) selection for diagnostic
algorithm to identify T1D subjects with fast renal decline.
T1D Combined Cohorts (n = 341)

| Protein Name | Model 1 | Model 2 | Model 3 |
|---|---|---|---|
| TNF-RSF1A | BF | BF | |
| TNF-RSF1B | | | |
| TNF-RSF3 | BF | BF | BF |
| TNF-RSF6B | BF | BF | BF |
| TNF-RSF7 | | | |
| TNF-RSF10A | BF | | |
| TNF-RSF4 | | | |
| TNF-RSF19L | BF | BF | BF |
| TNF-RSF27 | | | |
| CD160 | B | B | B |
| IL-1RT1 | BF | BF | |
| DLL1 | | | |
| LAYN | | B | B |
| PI3 | BF | BF | BF |
| NBL1 | BF | BF | |
| WFDC2 | | | |
| EFNA4 | | | |
| EPHA2 | BF | BF | BF |
| GFR-alpha-1 | | | |
| KIM1 | BF | BF | BF |
| eGFR* | | BF | BF |
| ACR* | | | BF |

The outcome is fast renal decline defined as an eGFR slope of between −80 ml/min/year and −5 ml/min/year. Logistic regression with was employed. The effects of proteins on the outcomes are estimated by per-quartile change.
*Inclusion of clinical covariates. eGFR at baseline. ACR—albumin to creatinine ratio in urine at baseline.

The Kidney Biomarker Panel 1 is a custom-made proteomic platform (OLINK) which allows for the measurement of absolute concentrations of the 21 informative proteins with very high precision and will be used to measure concentration of the informative proteins in about 5000 plasma/serum samples from several T1D cohorts. The results of these measurements will be used to develop a multi-marker diagnostic algorithm for the identification of subjects with T1D with fast renal decline, as well as a multi-marker prognostic algorithm to estimate the probability of development of ESRD in T1D subjects during 10 years of follow-up.

Further, preliminary data showed that the proteins included in the Kidney Biomarker Panel 1 are also important in T2D (data not shown).

### Example 5: Analysis of 1600 Subjects with T2D to Identify the Most Relevant Proteins from the Kidney Biomarker Panel 1

An exploratory study of 1600 subjects with T2D participating in the CRIC study (i.e., a discovery panel) will be used for the development diagnostic and prognostic algorithms to diagnose subjects with fast renal decline and to predict time of onset of ESRD. The subjects of this study had moderate renal impairment at entry and were followed for 5-10 years.

Measurements of the concentration of the 21 proteins using the Kidney Biomarker Panel 1 in baseline plasma specimens will be obtained from the CRIC study and analyzed using relevant epidemiological and statistical methods to evaluate associations of these proteins with: 1) fast renal decline and 2) time to onset of ESRD. More specifically, aliquots in amount of 20 μL of baseline plasma specimens from the T2D CRIC (n=1600) cohort, will be analyzed (OLINK laboratory) for measurements of the 21 informative proteins identified above and the data will be subjected to internal quality control (OLINK laboratory). Relevant clinical data from the T2D CRIC cohort will be obtained and analyzed using statistical analyses as described herein.

The data and statistical analyses will be used to develop a multi-marker diagnostic algorithm to diagnose subjects with fast renal decline (an eGFR slope of between −80 ml/min/year and −5 ml/min/year) and to develop a multi-marker prognostic algorithm to predict, with, e.g., an initial eGFR, the probability of developing ESRD during 5 years of observation, taking into account certain clinical characteristics (for example, including, some or all of the following, age, sex, ACR, eGFR, race) and concentrations of relevant proteins from the Kidney Biomarker Panel 1.

### Example 6: Validation of Results Obtained from the CRIC Study Using 1400 Subjects with T2D Participating from the Joslin Kidney Study (JKS)

The JKS cohort will be used to validate findings obtained in the CRIC study (Example 5). This validation panel will include a large cohort of T2D subjects who participated in the Joslin Kidney Study (JKS) and were followed for 9-15 years.

Data will be updated for the JKS T2D cohort to estimate eGFR slopes and ascertain onset of ESRD as of 2018. The JKS T2D cohort was updated Jun. 30, 2015. Since that update, 2100 additional plasma and urine specimens were collected and stored. Measuring serum creatinine and ACR in urine, clinical phenotyping regarding eGFR slopes will be improved in about 700 subjects. In addition, all subjects will be queried against rosters of the United States Renal Data System (USRDS) and the National Death Index (NDI) for the period 2015 through 2018. An additional 30 ESRD cases and 20 deaths will be identified.

Analyses will be performed to determine the plasma concentrations of the 21 proteins using the biomarker panel described herein in baseline plasma specimens from the JKS T2D cohort. More specifically, aliquots in amount of 20 μL of baseline plasma specimens from the T2D JKS (n=1400) cohort, will be analyzed (OLINK laboratory) for measurements of the 21 informative proteins identified above and the data will be subjected to internal quality control (OLINK laboratory). Relevant clinical data from the T2D JKS cohort will be obtained and analyzed using statistical analyses as described herein. External validation of the models described above will be performed using data obtained in the JKS T2D cohort.

### Example 7: Evaluation of Circulating WFDC2 and MMP-7 as Potential Indicators of Early Progressive Renal Decline

Study patients with type 2 diabetes (T2D) were enrolled in the Joslin Kidney Study. At enrollment, the patients (1,181 patients) had a normal baseline estimated glomerular filtration rate (eGFR)≥60 ml/min/1.73 m² (681 with normoalbuminuria and 500 with albuminuria) and at least a 6-year follow-up. Patients were followed for 6-12 years to ascertain fast renal decline defined as eGFR slope <−5 ml/min/1.73 m²/year.

The primary outcome was fast renal decline during 6-12 years of follow-up, defined as annual eGFR loss faster than or equal to −5 ml/min/1.73 m²/year. To estimate the slope of eGFR decline, the linear component of every patient's eGFR trajectory was extracted using patient-specific linear regression models.

During 6-12 years of follow-up, 152 (12.9%) patients developed fast renal decline defined as eGFR slope ≤−5 ml/min/1.73 m²/year (referred to in this study as decliners) and 33 (26%) of them developed ESKD. Characteristics of decliners versus non-decliners are shown in Table 8. At baseline decliners had higher systolic BP, were treated more frequently with angiotensin-converting enzyme inhibitor (ACE-I) and angiotensin II receptor blockers (ARB), had higher HbA1c and higher ACR and they had elevated levels of three markers; plasma TNF-R1 and KIM-1 and ratio of EGF/MCP-1 in urine. The two profibrotic markers, WFDC2 and MMP-7, measured in serum in this study were elevated in decliners and the differences between groups were highly statistically significant (Table 8).

TABLE 8
Comparison of clinical characteristics and biomarker levels in patients with type 2 diabetes
and eGFR ≥60 ml/min/1.73 m² who did and did not develop fast renal decline (n = 1,181).

| | All Patients | | Decliner | | Non-decliner | | P |
|---|---|---|---|---|---|---|---|
| | (N = 1,181) | | (N = 152) | | (N = 1,029) | | value |
| Characteristics at baseline | | | | | | | |
| Age, yr | 57 | (50; 62) | 57 | (51; 62) | 57 | (50; 61) | 0.176 |
| Men, % | 58 | | 64 | | 57 | | 0.096 |
| Duration of DM, yr | 10 | (6; 15) | 11 | (8; 17) | 10 | (6; 15) | 0.024 |
| BMI, kg/m² | 31 | (27; 36) | 32 | (29; 39) | 31 | (27; 36) | 0.012 |
| SBP, mmHg | 131 | (120; 142) | 139 | (127; 150) | 130 | (120; 141) | <.0001 |
| DBP, mmHg | 77 | (70; 83) | 79 | (71; 86) | 76 | (70; 83) | 0.010 |
| ACE-I or ARB Rx, % | 65 | | 78 | | 63 | | 0.0003 |
| Insulin Rx, % | 57 | | 57 | | 57 | | 0.965 |
| HbA_{1c}, % | 7.6 | (6.9; 8.6) | 8.1 | (7.0; 9.2) | 7.5 | (6.9; 8.5) | 0.0003 |
| eGFR, ml/min/1.73 m² | 96 | (83; 105) | 95 | (83; 102) | 96 | (83; 106) | 0.150 |
| Baseline ACR, mg/g | 13 | (6; 42) | 53 | (14; 415) | 11 | (6; 31) | <.0001 |
| Biomarkers at baseline | | | | | | | |
| plasma TNFR-1, pg/ml | 1250 | (1048, 16-3) | 1551 | (1186, 2022) | 1220 | (1030, 1535) | <.0001 |
| plasma KIM-1, pg/ml | 10 | (5; 17) | 16 | (10; 36) | 10 | (5; 16) | <.0001 |
| Urinary EGF/MCP-1 | 39 | (24; 76) | 25 | (14; 40) | 42 | (27; 83) | <.0001 |
| ratio | | | | | | | |
| Serum WFDC2, ng/ml | 6.7 | (5.6; 8.4) | 7.4 | (6.7; 10.7) | 6.7 | (5.4; 8.1) | <.0001 |
| Serum MMP-7, pg/ml | 3.9 | (3.0; 5.1) | 5.4 | (3.9; 7.1) | 3.8 | (2.9; 4.8) | <.0001 |
| During follow-up | | | | | | | |
| eGFR slope | −1.6 | (−3.3; −0.5) | −6.8 | (−9.5; −5.7) | −1.2 | (−2.4; −0.4) | <.0001 |
| (ml/min/1.73 m²/yr) | | | | | | | |
| Incidence of ESRD, n (%) | 33 | (2.7%) | 33 | (26%) | 0 | (0%) | <.0001 |
| Mortality, m (%) | 13 | (1%) | 4 | (3%) | 9 | (1%) | 0.053 |

Decliner is defined in this example as eGFR slope ≤−5 ml/min/1.73 m²/year calculated over a median 7-year follow-up. DM, diabetes mellitus. BMI, body mass index. SBP, systolic blood pressure. DBP, diastolic blood pressure. ACE-I, angiotensinconverting enzyme inhibitor. ARB, angiotensin II receptor blocker. Rx, treatment. eGFR, estimated glomerular filtration. ACR, albumin creatinine ratio. ESKD, end-stage kidney disease.

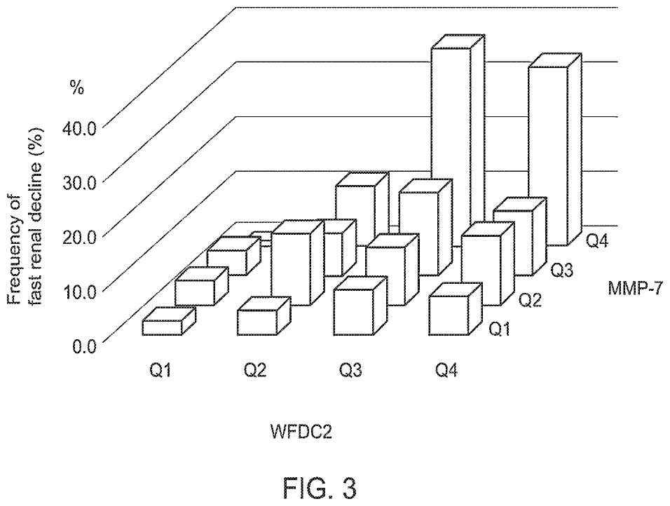

Levels of the profibrotic proteins were only weakly correlated with the other markers and moderately with TNF-R1 and between themselves (Table 9). Table 9 shows Spearman rank correlation coefficients matrix of clinical characteristics and biomarkers. The observed proportion of fast renal decline according to baseline levels of quartiles of the two profibrotic proteins is shown in FIG. 3. Individuals with low baseline values of WFDC2 (Q1) and low values of MMP-7 (Q1) had a very low risk of fast renal decline during 6-12 years of follow-up. In contrast patients with high baseline levels of both profibrotic proteins (WFDC2 and MMP-7) had risk for fast renal decline increasing in a multiplicative way. Overall these two proteins showed largely independent effects.

The data demonstrate that the elevation of circulating WFDC2 and MMP-7 strongly contribute to early progressive renal decline in type 2 diabetes.

TABLE 9
Spearman rank correlation coefficients matrix of clinical characteristics and biomarkers

| | Systolic | | | Urinary | Urinary | Plasma | Plasma | Serum | Serum |
|---|---|---|---|---|---|---|---|---|---|
| | BP | HbA_{1c} | eGFR | ACR | EGF/MCP-1 | KIM-1 | TNF-R1 | WFDC2 | MMP-7 |
| eGFP slope | −0.16*** | −0.11** | −0.02 | −0.27*** | 0.24*** | −0.20*** | −0.22*** | −0.20*** | −0.27*** |
| Systolic BP | | 0.08** | −0.05 | 0.15*** | −0.10** | 0.06* | 0.11** | 0.05 | 0.09** |
| HbA_{1c} | | | 0.12*** | 0.14*** | −0.12*** | 0.14*** | 0.07* | 0.01 | 0.002 |
| eGFP | | | | 0.03 | 0.21*** | −0.13*** | −0.45*** | −0.47*** | −0.34*** |
| Urinary ACR | | | | | −0.28*** | 0.31*** | 0.32*** | 0.23*** | 0.35*** |
| Urinary EGF/MCP-1 | | | | | | −0.23*** | −0.32*** | −0.32*** | −0.36*** |
| Plasma KIM-1 | | | | | | | 0.27*** | 0.22*** | 0.31*** |
| Plasma TNF-R1 | | | | | | | | 0.58*** | 0.47*** |
| Serum WFDC2 | | | | | | | | | 0.58*** |

P-values
*<.05,
**<.01,
***<0.0001

### Example 8: Development of Algorithms to Classify Subjects with Fast Renal Decline and High Probability of Progression to ESRD

Models developed and described herein will be used to develop algorithms for identifying subjects with fast renal decline and high probability of progression to ESRD. Based on data described herein, a combination, or combinations, of certain biomarkers (e.g., at least 2 biomarkers, at least 3 biomarkers, at least 4 biomarkers, at least 5 biomarkers, at least 6 biomarkers, at least 7 biomarkers, at least 8 biomarkers, at least 9 biomarkers, at least 10 biomarkers, at least 11 biomarkers, at least 12 biomarkers, at least 13 biomarkers, at least 14 biomarkers, at least 15 biomarkers, at least 16 biomarkers, at least 17 biomarkers, at least 18 biomarkers, at least 19 biomarkers, at least 20 biomarkers, at least 21 biomarkers, at least 22 biomarkers, at least 23 biomarkers, at least 24 biomarkers, at least 25 biomarkers, at least 26 biomarkers, at least 27 biomarkers, at least 28 biomarkers, at least 29 biomarkers, etc.) strongly and independently associated with renal decline contribute to the development of diagnostic and prognostic algorithms. While it is possible that certain biomarkers may provide some redundant information due to high correlation with other proteins identified by the methods and compositions described herein, the joint distribution of these proteins, weighted by their independent strength of association, will be used to construct a diagnostic score that will be able to classify subjects into groups of slow and fast renal decline. To predict the time and likelihood of developing ESRD, for example, within the next five years, the diagnostic score constructed to identify subjects with fast renal decline will be re-calculated to accommodate for predictive purposes. The time to ESRD onset depends not only on the rate of renal decline (i.e., the slope), but may also on the baseline renal function (the intercept), and possible deviations from the linear model of the decline, as well as certain clinical characteristics, such as sex, age, ACR and others, as well as the risk of mortality prior to ESRD (considered herein as a competing risk), lowering one's chances of surviving until kidney failure. In such a complex scenario, the contributions of each of the proteins from the Kidney Biomarker Panel 1 will be assessed to best predict the time of onset of ESRD. Finally, the diagnostic and prognostic algorithms may take the form of a computer-based calculator.

### Example 9: Comprehensive Search for Axon Guidance Pathway (AGP) Proteins Associated with Risk of ESKD in Diabetes

This example describes the identification of a specific set of circulating axon guidance pathway (AGP) proteins that is associated with risk of ESKD (ESRD) and with severity of the kidney structural lesions observed in kidney biopsy specimens.

### Methods

In order to identify markers that could be generalized to subjects with diabetes, profiles of axon guidance pathway (AGP) proteins in four independent cohorts with type 1 diabetes (T1D) and type 2 diabetes (T2D), who were at various stages of DKD, and were followed for 7-15 years to ascertain progression to ESKD were studied. A subgroup of 105 subjects in the Pima T2D validation cohort had research kidney biopsies in close proximity to the baseline examination. This subgroup was used for studies on the relationships between kidney structural lesions, kidney expression of genes encoding the AGP proteins, and circulating levels of AGP proteins. Subjects for the study cohorts were selected from the Joslin Kidney Study (JKS) and the Pima Indian Kidney Study.

### JKS (Joslin Kidney Study)

JKS is a longitudinal, observational study that investigates the determinants and natural history of kidney function decline in T1D and T2D. Approximately 2000 subjects with T1D and 1500 subjects with T2D were recruited into JKS from the 20,000 subjects attending the Joslin Clinic between 1991 and 2009. The protocols for recruitment, examination, and determination of clinical characteristics for these subjects were previously reported (21-26).

Discovery and Replication Cohorts with Late DKD

The T1D discovery (n=239) and the T2D replication (n=136) cohorts were selected from JKS participants, with proteinuria and impaired kidney function (eGFR of 20-60 ml/min per 1.73 m²) at baseline, who enrolled in the study between 1991 and 2009. These subjects were followed for 7-15 years to assess changes in kidney function and ascertain onset of ESKD. Subjects in these cohorts were previously used in our study on the role of inflammatory circulating proteins in progression to ESKD (32).

Validation Cohort with Early DKD

The T1D validation cohort (n=243) included JKS participants with proteinuria and an eGFR of 60-140 ml/min per 1.73 m² at baseline, who were enrolled into the study between 1991 and 2009. These subjects were followed for 7-15 years to assess changes in kidney function and ascertain onset of ESKD. Subjects in this cohort were previously used in a study on the role of circulating TNF-related proteins in the development of early progressive renal decline (33).

### Pima Indian Kidney Study

The T2D validation cohort was selected from the subjects participating in the Pima Indian Kidney Study (27-29). For the T2D validation cohort, 154 of these subjects with a baseline GFR of >60 ml/min were selected. Research kidney biopsies were performed in 105 of these subjects in close proximity to their baseline examinations. Subjects in these cohorts were previously used in a study on the role of inflammatory circulating proteins in progression to ESKD (32).

### SOMAscan: Aptamer-Based Proteomics Platform

Unique, single-stranded sequences of DNA and RNA, referred to as aptamers, recognize folded protein epitopes with high affinity and specificity (30, 31). This property was further advanced by using slow off-rate modified aptamers to develop the SOMAscan platform (SomaLogic, Denver, CO), which we used to assay concentrations of proteins in this study (32-34). All samples in this study were processed at the SomaLogic laboratory in Denver.

### Olink: Proximity Extension Assay-Based Proteomics Platform

To validate the SOMAscan measurements of AGP proteins, the Olink Proteomics platform was used, which applies the proximity extension assay to measure the concentration of 1100 proteins (35). By comparing the plasma concentration of four AGP proteins measured by the SOMAscan and Olink platform in 75 subjects from the Pima Indian cohort, we found good agreement between the measurements (Spearman correlation coefficients varied between 0.56 and 0.68; see Table 19). Furthermore, hazard ratios (HRs) for time to onset of ESKD, according to baseline concentration of the AGP proteins measured by SOMAscan and Olink platforms, were similar and statistically significant for two proteins (EPHA2 and EPHB6). When measured by SOMAscan, EFNA4 resulted in a lower and non-significant HR compared with the measurement obtained using the Olink platform; measuring UNC5C using the Olink platform resulted in a lower and non-significant HR compared with that obtained from SOMAscan (see Table 19). These differences were small and insignificant, and were likely due to the size of the study group. In addition to the validation study, in which commercially available Olink panels were used, a custom-made Olink assay was designed to measure concentrations of EFNA4 and EPHA2 in the serum from participants from the Action to Control Cardiovascular Risk in Diabetes (ACCORD) trial, and also from the cell lysates and supernatant (medium in which the cells were cultured) in in vitro studies.

### Measurements of Kidney Structural Lesions

The morphometric techniques were described previously (38, 54). Briefly, unbiased random samplings of tissue sections from kidney biopsies provided digital images from both electron and light microscopy. Quantitative morphometric methods were used to estimate kidney structural parameters by masked observers. The predefined electron microscopy parameters included glomerular basement membrane (GBM) width, mesangial fractional volume (VvMes), glomerular filtration surface area density, podocyte number per glomerulus, podocyte foot process width, percentage of GBM surface with podocyte detachment, and percentage of normally fenestrated glomerular capillary endothelium (% FE). Cortical interstitial fraction volume (VvInt) was measured using light microscopy and, because not all participants had tissue for VvInt, data for this parameter were only available for 102 subjects. Foot process width, percentage of GBM surface with podocyte detachment, and % FE were not measured in one participant. Generally, three glomeruli (or a minimum of two) were examined by electron microscopy per participant, and a median of seven (interquartile range, 3-12) glomeruli were examined by light microscopy for the morphometric measurements. Morphometric variables for each individual were calculated as the mean of all measurements for that individual.

mRNA Expression of Gene Encoding for AGP Proteins in Kidney Tissue

Of the 105 Pima subjects who had structural measurements, 66 also had gene expression profiling of the glomerular tissue, and 47 had gene expression profiling of the tubulointerstitium for mRNA expression, using a GeneChip Human Genome series U133A and Plus 2.0 Array (Affymetrix, Santa Clara, CA) (36-39). mRNAs for five of the six AGP proteins were identified.

### Immunohistochemical Staining of Kidney Tissue for AGP Proteins

Paraffin sections from two kidney biopsy specimens from the Pima Indian cohort were stained for EFNA4 and EPHA2, using primary antibodies, and the images were obtained using a Leica SP8X confocal microscope.

### ACCORD Study

The ACCORD study was a multicenter clinical trial that investigated the effect of treating hyperglycemia, hypertension, and dyslipidemia on cardiovascular event rates. The trial included 10,251 participants with T2D and high cardiovascular risk, who were randomized in a 1:1 ratio to receive intensive (aiming for hemoglobin A1c [HbA1c], 6.0%) or standard (aiming for HbA1c level of 7%-7.9%) glycemic-lowering treatment (40). This study included 200 of the ACCORD-Lipid (one of the ACCORD study subtrials) participants, who were randomly selected in equal numbers from the four groups defined by the two glycemic treatment arms and the two lipid treatment arms. The resulting sample comprised 100 participants from the intensive glycemic arm and 100 participants from the standard glycemic arm, each including an equal number of subjects from each lipid-treatment arm (fenofibrate or placebo). Using the Olink Proteomics platform described above, EFNA4 and EPHA2 were measured in serum samples obtained at baseline and 12 months after randomization.

### Cellular Studies in Renal Proximal Tubule Epithelial Cells, Fibroblasts, and Human Umbilical Vein Endothelial Cells

AGP protein expression levels were assessed in cell lysates and supernatant in three human cell lines: renal proximal tubule epithelial cells (RPTECs), fibroblasts, and human umbilical vein endothelial cells (HUVECs). RPTECs/TERT1 (CRL-4031) were obtained from American Type Culture Collection (Manassas, VA) and cultured according to the manufacturer's protocol. Skin fibroblasts from subjects with T1D were provided by M. M. and cultured as previously describe (41). HUVECs were isolated from the umbilical cords of newborns and cultured as previously described (42, 43). The cell lysates and supernatants from the three cell lines were analyzed using the custom-made Olink assay to measure levels of EPHA2 and EFNA4.

### Statistical Analyses

Clinical characteristics and summaries of outcomes were reported as counts and percentages (proportions) for categoric variables, means with SDs for normally distributed continuous variables, and as medians and quartiles for variables with skewed distributions.

To further examine the association of the AGP proteins with the risk of ESKD, the study fitted simple (univariate) and adjusted (multivariable) Cox proportional hazards regression models. In these analyses, to make the results of this study comparable with recent proteomics publications, 12, 13, 15 the levels of AGP proteins at baseline were modeled as one-quartile changes on a continuous scale. In some instances, results were presented with baseline quartiles considered as discrete variables, or compared risk of progression to ESKD in subjects in the fourth quartile versus those in the first quartile for specific AGP proteins. The analyses for AGP proteins were repeated using raw data with changes modeled as one SD.

Two multivariable Cox proportional hazard regression models were used to assess the role of circulating miRNAs and AGP proteins in progression to ESKD. In the models referred to as “etiologic,” the effects of AGP proteins were adjusted for significant clinical exposures and confounders, such as sex, diabetes duration, systolic BP, baseline eGFR (or directly measured GFR, where applicable), HbA1c, and stratification by study cohorts. The urine albumincreatinine ratio (ACR) was not considered as a covariate in these models because it was an outcome measure and a symptom of the DKD presentation that leads to ESKD. However, in the so-called multivariable “prognostic” models, in which AGP proteins were assessed as predictors of progression to ESKD, clinical predictors such as ACR, HbA1c, and eGFR were also considered. Effects of clinical covariates on progression to ESKD in the two models are shown in Table 20. Enrichment pathway analysis of the miRNA gene/protein targets was performed with a two-sided Fisher exact test against the null hypothesis of no enrichment. The association of the AGP proteins with 10-year risk of ESKD was tested with adjusted Cox regression models. We applied a Bonferroni correction for n=1128 independent tests (the number of proteins measured) performed in the T1D discovery cohort, which yielded a threshold of P<4.4×10⁻⁵.

Spearman correlation was used to examine association of early structural kidney lesions with kidney expression of miRNAs and AGP genes and circulating AGP proteins.

We examined the performance of two prognostic models using subjects in four study cohorts. Using a Cox proportional hazards model and backward variable elimination, a clinical prognostic model was developed that included clinical covariates such as ACR, HbA1c, and eGFR. The prognostic performance of this model was compared with the prognostic performance of a model that included the three significant clinical covariates and two of the six AGP proteins. Several different methods were used to assess prognostic performances of the two models. The Uno C-statistic was calculated as proportions of pairs for subjects whose observed and predicted outcomes were concordant. The fit of the consecutive nested models was tested using likelihood ratio tests, with the appropriate degrees of freedom and an assumption of chi-squared distribution, and by changes in the Akaike information criterion (AIC) values. To evaluate overall improvement in reclassification, net reclassification improvement (NRI) was used on the basis of several ESKD risk categories (5%, 5%-9.9%, 10%-19.9%, and >20%).

In ACCORD, the effect of intensive glycemic treatment on serum EFNA4 and EPHA2 concentrations was evaluated by linear regression. Due to their right-skewed distributions, both proteins were analyzed after log transformation. For each protein, the linear regression model included the protein concentration at 12 months as the dependent variable, and the glycemic treatment assignment as the independent variable, with the protein concentration at baseline, age, sex, eGFR at baseline and 12 months, and lipid-treatment assignment as covariates. The mean covariate-adjusted ratios between 12 months and baseline protein values were estimated for the two treatment arms by linear regression.

The effect of losartan versus placebo treatment on serum EFNA4 and EPHA2 concentrations by SOMAscan in a subset of the Pima cohort was also evaluated by linear regression. Both proteins were analyzed after log transformation, and 12-month changes were calculated, adjusting for duration of diabetes, sex, systolic BP, baseline GFR, and HbA1c.

All in vitro experiments were performed in triplicate, and the results are expressed as means6SEMs. One-way ANOVA with the Dunnett multiple comparison test was used for statistical analysis. Analyses were performed in SAS version 9.4 and R version 3.2.4.

### Results

### Clinical Characteristics of Study Cohorts

To obtain robust findings that can be generalized to all subjects with diabetes, several different cohorts were selected for this study. This included two cohorts with impaired kidney function (late DKD), and two cohorts with normal kidney function (early DKD). The cohort with T1D and late DKD was considered a discovery cohort, the cohort with T2D and late DKD was considered a replication cohort, and the two cohorts with early DKD were considered validation cohorts. Clinical characteristics of these cohorts are shown in Table 10. They differed with regard to baseline clinical characteristics, such as age, diabetes duration, HbA1c, ACR, and eGFR. However, a significant proportion of subjects in each cohort developed ESKD during the 7-15 years of follow-up. Progression to ESKD during the first 10 years of follow-up was considered the primary outcome in this study. In the Joslin cohorts, 88% of subjects were of European ancestry, whereas the Pima cohort all subjects were American Indians.

TABLE 10
Clinical characteristics of subjects included in the study cohorts

| | | | Early DKD | | |
|---|---|---|---|---|---|
| | Late DKD | | | | Pima T2D |
| | Joslin T1D | Joslin T2D | Joslin T1D | Pima T2D | Cohort for |
| | Discovery | Replication | Validation | Validation | Auxiliary |
| | Cohort | Cohort | Cohort | Cohort | Study |
| Characteristics | (n = 239)^{a} | (n = 136)^{a} | (n = 243)^{b} | (n = 154)^{a} | (n = 105)^{c} |
| At baseline | | | | | |
| Male/female, n | 117/122 | 89/47 | 133/110 | 43/111 | 29/76 |
| Age (yr), | 44 ± 10 | 60 ± 6 | 39 ± 9 | 46 ± 10 | 45 ± 10 |
| mean ± SD | | | | | |
| Duration of DM | 30 ± 9 | 16 ± 9 | 26 ± 9 | 16 ± 7 | 15 ± 6 |
| (yr), mean ± SD | | | | | |
| HbA_{1c} | 8.6 ± 1.6 | 7.5 ± 1.8 | 9.0 ± 1.7 | 9.4 ± 2.3 | 9.2 ± 2.3 |
| (%), mean ± SD^{d} | | | | | |
| SBP (mmHg), | 136 ± 20 | 139 ± 19 | 131 ± 19 | 125 ± 14 | 122 ± 13 |
| mean ± SD | | | | | |
| DBP (mmHg) | 77 ± 11 | 75 ± 11 | 78 ± 10 | 77 ± 9 | 77 ± 9 |
| mean ± SD | | | | | |
| ACR (mg/g), | 793 (274- | 255 (57- | 579 (212- | 55 (13- | 40 (12- |
| median (IQR) | 1855) | 1092) | 1173) | 357) | 124) |
| GFR (ml/min), | 42 ± 10 | 49 ± 11 | 97 ± 21 | 150 ± 47 | 151 ± 47 |
| mean ± SD | | | | | |
| During follow-up | | | | | |
| New cases of | 127 (53) | 43 (32) | 50 (21) | 37 (24) | 15 (14) |
| ESKD with 10 | | | | | |
| yr, n (%) | | | | | |

DM, diabetes mellitus; SBP, systolic BP; DBP, diastolic BP; IQR, interquartile range.
^{a}These study cohorts were used to study KRIS proteins, and their clinical characteristics were previously reported (23).
^{b}This study cohort was used to study TNF-related proteins, and clinical characteristics were previously reported (24).
^{c}This subgroup from the Pima T2D validation cohort had kidney biopsies and was used to study kidney structural lesions.
^{d}HdA1c was standardized to Diabetes Control and Complications Trial units (25, 29).
^{e}In the Joslin cohorts, eGFR was determined using serum creatinine concentrations (25). In the Pima cohort, GFR (ml/min) was directly measured by urinary clearance of iothalamate (29).

### AGP Proteins and Risk of ESKD

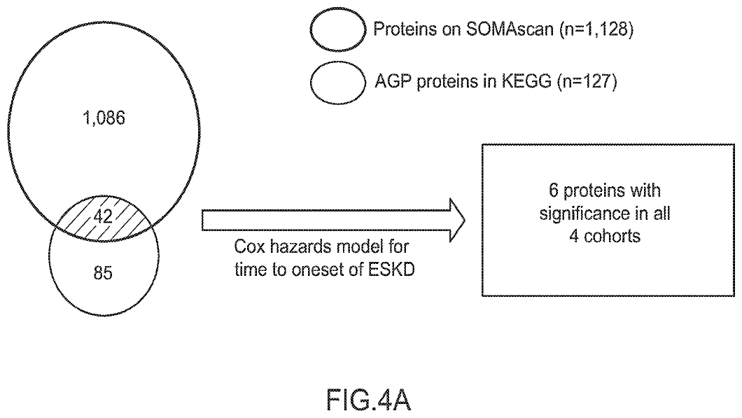

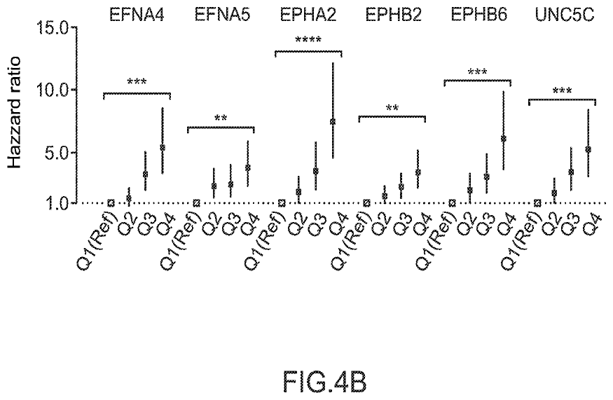

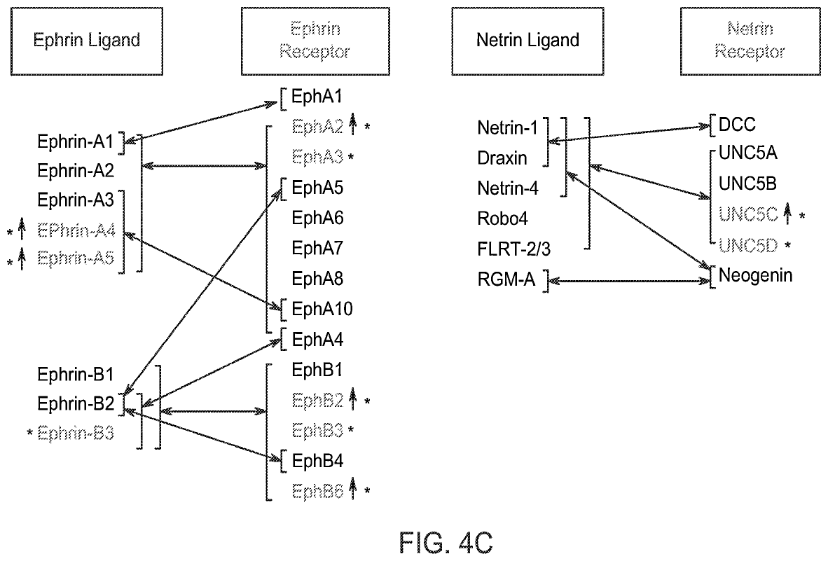

AGP was the top candidate pathway identified through bioinformatics analysis. Of the 127 proteins in this pathway listed in KEGG, the concentrations of 42 proteins were measured at baseline in circulation using the SOMAscan proteomics platform (FIG. 4A). Of these proteins, 36 did not show association with time to onset of ESKD in Cox regression analyses (Table 12). Those that showed an increase are described in Table 11.

TABLE 11
Hazard Ratios per 1-SD increase in AGP proteins levels
for time to onset of ESKD during a 10-year follow-up.

| | Late DKD | | | | Early DKD | | | | | |
|---|---|---|---|---|---|---|---|---|---|---|
| | T1D Discovery | | T2D Replication | | T1D Validation | | T2D Validation | | Combined | |
| | cohort (n = 213) | | cohort (n = 136) | | cohort (n = 243) | | cohort (n = 153) | | cohorts (n = 745) | |
| Gene | HR | P | HR | P | HR | P | HR | P | HR | P |
| EFNA4 | 2.13 | 3.0 × 10⁻¹⁵ | 1.69 | 7.7 × 10⁻⁴ | 2.02 | 3.5 × 10⁻⁶ | 1.73 | 2.2 × 10⁻³ | 1.91 | 1.3 × 10⁻¹⁸ |
| EFNA5 | 1.86 | 3.2 × 10⁻⁹ | 1.85 | 1.1 × 10⁻⁴ | 1.58 | 9.7 × 10⁻⁴ | 2.29 | 1.7 × 10⁻⁵ | 1.67 | 2.3 × 10⁻¹² |
| EPHA2 | 2.40 | 1.1 × 10⁻¹⁵ | 2.23 | 2.9 × 10⁻⁶ | 1.84 | 3.5 × 10⁻⁵ | 2.02 | 7.2 × 10⁻⁵ | 2.29 | 2.1 × 10⁻²⁵ |
| EPHB2 | 1.58 | 5.7 × 10⁻⁶ | 1.52 | 6.6 × 10⁻³ | 1.40 | 1.8 × 10⁻² | 1.66 | 3.3 × 10⁻³ | 1.64 | 3.1 × 10⁻¹² |
| EPHB6 | 1.84 | 3.1 × 10⁻⁹ | 1.97 | 8.3 × 10⁻⁶ | 1.51 | 4.9 × 10⁻³ | 2.23 | 9.0 × 10⁻⁶ | 1.99 | 1.5 × 10⁻¹⁷ |
| UNC5C | 2.22 | 2.9 × 10⁻¹³ | 1.59 | 2.9 × 10⁻³ | 1.89 | 3.7 × 10⁻⁵ | 1.67 | 3.3 × 10⁻³ | 1.91 | 1.1 × 10⁻¹⁶ |

Results are presented for individual and combined cohorts for etiologic model.
*Etiologic model was adjusted for sex, duration of diabetes, systolic blood pressure, HbA1c and eGFR with stratification by variable indicating cohort.
HR was computed for a 1-SD increase in concentration of AGP proteins.
HR—Hazard ratio.

TABLE 12
List of 36 AGP proteins present on SOMAscan platform that were not
associated with risk of ESKD or not confirmed in study cohorts.

| | | HR per 1- | | HR per 1- | | |
|---|---|---|---|---|---|---|
| Gene | Target proteins | quartile^{#} | P* | SD^{##} | P | Other cohorts |
| EPHA1 | Ephrin type-A receptor 1 | 1.52 | 2.0 × 10⁻⁶ | 1.60 | 1.3 × 10⁻⁶ | Not confirmed |
| UNC5D | Netrin Receptor UNC5D | 1.48 | 1.4 × 10⁻⁵ | 1.65 | 1.7 × 10⁻⁶ | Not confirmed |
| EPHA5 | Ephrin type-A receptor 5 | 1.34 | 8.5 × 10⁻⁴ | 1.31 | 3.7 × 10⁻³ | — |
| EPHB4 | Ephrin type-B receptor 4 | 0.79 | 7.4 × 10⁻³ | 0.82 | 3.5 × 10⁻² | — |
| EFNB3 | Ephrin-B3 | 1.25 | 9.4 × 10⁻³ | 1.23 | 2.2 × 10⁻² | — |
| PPP3R1 | Calcineurin B alpha isoform | 0.77 | 2.2 × 10⁻² | 0.74 | 2.6 × 10⁻² | — |
| PAK5 | PAK7 | 0.77 | 2.6 × 10⁻² | 0.77 | 5.3 × 10⁻² | — |
| CXCL12 | SDF-1 | 0.85 | 5.0 × 10⁻² | 0.80 | 2.5 × 10⁻² | — |
| SEMA3A | Semaphorin 3A | 1.17 | 6.5 × 10⁻² | 1.14 | 1.7 × 10⁻¹ | — |
| RAC1 | RAC1 | 0.86 | 7.8 × 10⁻² | 0.85 | 7.1 × 10⁻² | — |
| SEMA6A | Semaphorin-6A | 1.17 | 7.8 × 10⁻² | 1.18 | 9.0 × 10⁻² | — |
| NRP1 | NRP1 | 1.23 | 9.4 × 10⁻² | 1.20 | 1.8 × 10⁻¹ | — |
| CFL1 | Cofilin-1 | 0.87 | 1.1 × 10⁻¹ | 0.87 | 1.4 × 10⁻¹ | — |
| NTN4 | NET4 | 1.21 | 1.1 × 10⁻¹ | 1.27 | 8.9 × 10⁻² | — |
| MAPK1 | MK01 | 0.88 | 1.2 × 10⁻¹ | 0.89 | 2.1 × 10⁻¹ | — |
| GSK3B | GSK-3 beta | 0.88 | 1.3 × 10⁻¹ | 0.81 | 3.2 × 10⁻² | — |
| FYN | FYN | 0.89 | 1.6 × 10⁻¹ | 0.90 | 2.6 × 10⁻¹ | — |
| PPP3CA | Calcineurin | 0.90 | 1.7 × 10⁻¹ | 0.90 | 2.3 × 10⁻¹ | — |
| PPP3R1 | Calcineurin | 0.90 | 1.7 × 10⁻¹ | 0.90 | 2.3 × 10⁻¹ | — |
| PAK6 | PAK6 | 0.86 | 1.8 × 10⁻¹ | 0.87 | 2.6 × 10⁻¹ | — |
| PTK2 | FAK1 | 0.86 | 1.9 × 10⁻¹ | 0.80 | 1.1 × 10⁻¹ | — |
| MAPK3 | ERK-1 | 0.90 | 2.1 × 10⁻¹ | 0.90 | 2.6 × 10⁻¹ | — |
| SEMA3E | Semaphorin 3E | 0.88 | 2.7 × 10⁻¹ | 0.82 | 1.7 × 10⁻¹ | — |
| RASA1 | RASA1 | 0.89 | 3.3 × 10⁻¹ | 0.87 | 2.7 × 10⁻¹ | — |
| KRAS | K-ras | 1.08 | 5.2 × 10⁻¹ | 1.15 | 3.4 × 10⁻¹ | — |
| MET | Met | 0.95 | 5.3 × 10⁻¹ | 0.92 | 3.7 × 10⁻¹ | — |
| ROBO3 | ROBO3 | 0.95 | 6.8 × 10⁻¹ | 0.98 | 8.7 × 10⁻¹ | — |
| ROBO2 | ROBO2 | 1.03 | 7.0 × 10⁻¹ | 1.02 | 8.2 × 10⁻¹ | — |
| NCK1 | NCK1 | 0.97 | 7.7 × 10⁻¹ | 0.98 | 8.5 × 10⁻¹ | — |
| PAK3 | PAK3 | 1.02 | 8.0 × 10⁻¹ | 1.04 | 6.9 × 10⁻¹ | — |
| PLXNC1 | PLXC1 | 1.03 | 8.4 × 10⁻¹ | 1.02 | 8.9 × 10⁻¹ | — |
| L1CAM | NCAM-L1 | 0.99 | 8.8 × 10⁻¹ | 0.96 | 6.4 × 10⁻¹ | — |
| ABL1 | ABL1 | 1.01 | 9.1 × 10⁻¹ | 1.00 | 9.7 × 10⁻¹ | — |
| EPHA3 | Ephrin type-A receptor 3 | 1.01 | 9.4 × 10⁻¹ | 0.96 | 6.9 × 10⁻¹ | — |
| ITGB1 | Integrin beta-1 | 1.00 | 9.9 × 10⁻¹ | 1.03 | 7.3 × 10⁻¹ | — |
| CDK5 | CDK5 | 1.00 | 1 | 0.99 | 9.1 × 10⁻¹ | — |

^{#}HR per 1-quartile increase in concentration of AGP proteins was considered.
^{##}HR per 1-SD increase was considered.
*Bonferroni correction for the available proteins on the SOMAscan platform was applied for the T1D Discovery cohort (P < 4.4 × 10⁻⁵).

However, the concentrations of six of these proteins—EFNA4, EFNA5, EPHA2, EPHB2, EPHB6, and UNC5C—were associated with time to onset to ESKD during 10-year follow-up in each of the different study cohorts, i.e., in cohorts with late and early DKD, T1D and T2D, and in White and Pima Indian subjects. Table 13 shows the HRs for time to onset of ESKD. Univariate HRs according to quartiles (continuous variable) of the concentrations of six AGP proteins were very similar and statistically significant among the individual study cohorts. When the cohorts were combined and adjusted for clinical covariates relevant for the etiologic model, the HRs became extremely highly statistically significant. The relationship between quartiles (discrete variable) of concentrations of the six AGP proteins and HRs for time to onset of ESKD is shown in FIG. 4B and Table 13. In comparison with the first quartile (reference), the HR for time to onset of ESKD in each subsequent quartile increased in a dose-response manner (FIG. 4B). The steepest increase in HRs was observed in the quartiles of concentrations of EPHA2. The HRs for time to onset of ESKD between subjects in the first quartile and the fourth quartile were large and highly statistically significant for all six of the proteins. When a similar analysis was performed for the prognostic model that included ACR, the HRs for each of the six AGP proteins diminished; however, they remained large and statistically significant (Table 13).

TABLE 13
HRs for time to onset of ESKD during a 10-year follow-up

| | Etiologic Models In Individual Cohorts, | | | | | | | | Etiologic Models in | | | | Prognostic | |
|---|---|---|---|---|---|---|---|---|---|---|---|---|---|---|
| | With Quartiles as Continuous Variables | | | | | | | | Combined Cohorts^{a} | | | | Models^{b} | |
| | Late DKD | | | | Early DKD | | | | Quartiles as | | Quartiles as | | Quartiles as | |
| | T1D Discovery | | T2D Replication | | T1D Validation | | T2D Validation | | Continuous | | Discrete Values, | | Discrete Values, | |
| | Cohort | | Cohort | | Cohort | | Cohort | | Values | | Comparison of | | Comparison of | |
| | (n = 213) | | (n = 136) | | (n = 243) | | (n = 154) | | (n = 745) | | Q4 versus Q1 | | Q4 versus Q1 | |
| Gene | HR | P | HR | P | HR | P | HR | P | HR | P | HR | P | HR | P |
| EFNA4 | 2.10 | 2.4 × 10⁻¹³ | 1.61 | 1.0 × 10⁻³ | 1.98 | 4.1 × 10⁻⁶ | 1.65 | 1.9 × 10⁻³ | 1.84 | 6.4 × 10⁻¹⁸ | 5.36 | 6.0 × 10⁻¹³ | 3.35 | 2.5 × 10⁻⁷ |
| EFNA5 | 1.70 | 1.8 × 10⁻⁸ | 1.64 | 7.6 × 10⁻⁴ | 1.40 | 1.1 × 10⁻² | 1.89 | 1.6 × 10⁻⁴ | 1.45 | 9.7 × 10⁻⁹ | 3.75 | 1.1 × 10⁻⁸ | 2.21 | 7.2 × 10⁻⁴ |
| EPHA2 | 2.15 | 1.4 × 10⁻¹⁴ | 1.89 | 3.0 × 10⁻⁵ | 1.68 | 2.5 × 10⁻⁴ | 2.00 | 7.3 × 10⁻⁵ | 1.99 | 4.5 × 10⁻²² | 7.48 | 4.8 × 10⁻¹⁶ | 4.24 | 1.4 × 10⁻⁸ |
| EPHB2 | 1.45 | 3.2 × 10⁻⁻⁵ | 1.45 | 8.6 × 10⁻³ | 1.29 | 4.6 × 10⁻² | 1.62 | 2.8 × 10⁻³ | 1.51 | 1.7 × 10⁻¹⁰ | 3.41 | 5.6 × 10⁻⁹ | 2.03 | 5.9 × 10⁻⁴ |
| EPHB6 | 1.74 | 3.7 × 10⁻⁹ | 1.88 | 2.3 × 10⁻⁵ | 1.54 | 2.0 × 10⁻³ | 2.12 | 2.2 × 10⁻⁵ | 1.78 | 6.1 × 10⁻¹⁶ | 6.07 | 2.1 × 10⁻¹³ | 2.86 | 2.5 × 10⁻⁵ |
| UNC5C | 1.83 | 1.3 × 10⁻¹⁰ | 1.59 | 1.5 × 10⁻³ | 1.91 | 1.3 × 10⁻⁵ | 1.56 | 4.4 × 10⁻³ | 1.73 | 3.4 × 10⁻¹⁵ | 5.22 | 5.3 × 10⁻¹² | 2.94 | 1.0 × 10⁻⁵ |

HRs were determined according to baseline levels of six AGP proteins and expressed as quartiles, which were considered as either continues or discrete values. Results are presented for individual and combined cohorts for etiologic and prognostic models. Results of analyses according to baseline levels of six AGP proteins expressed as one SD change is shown in Table 11. HR per one-quartile change when quartiles were considered as continuous variable. Q4, quartile 4; Q1, quartile 1.
^{a}Etiologic models were adjusted for sex, duration of diabetes, systolic BP, HbA1c, and eGFR, with variable stratification by study cohort. One subject who had incomplete clinical information was excluded in the combined model, and 745 subjects were used for subsequent analyses.
^{b}Prognostic models were adjusted for baseline HbA1c, eGFR, and ACR, with variable stratification by study cohort.

In the analysis in which all six AGP proteins were analyzed together with clinical covariates relevant for the etiologic model, only the EPHA2 receptor and EFNA4 ligand showed independent associations with time to onset of ESKD (HR for EPHA2, 1.67; 95% CI, 1.37 to 2.03; P=3.9×10⁻⁷; HR for EFNA4, 1.27; 95% CI, 1.04 to 1.55; P=1.7×10⁻²). The results of similar analyses for the prognostic model are shown in Table 14.

TABLE 14
Comparison of performance of a prognostic model that included only clinical covariates
versus a prognostic model that included clinical covariates and AGP proteins

| Models | | | |
|---|---|---|---|
| Only Clinical | | Clinical and AGP Proteins | |
| Statistic | P Value | Statistic | P Value |

Effect estimates, HR (95% CI)^{a}

| | | | | |
|---|---|---|---|---|
| HbA1c (%) | 1.25 (1.16 to 1.34) | 6.8 × 10⁻¹⁰ | 1.31 (1.33 to 1.41) | 1.2 × 10⁻¹³ |
| eGFR per 10 | 0.85 (0.79 to 0.92) | 2.4 × 10⁻⁵ | 0.93 (0.86 to 1.00) | 4.0 × 10⁻² |
| ml/min per 1.73 m² | | | | |
| Log₂ACR | 1.37 (1.28 to 1.46) | 1.6 × 10⁻²² | 1.28 (1.20 to 1.36) | 1.1 × 10⁻¹³ |
| EFNA4 per quartile | | | 1.27 (1.03 to 1.56) | 2.4 × 10⁻² |
| EPHA2 per quartile | | | 1.42 (1.16 to 1.75) | 7.9 × 10⁻⁴ |
| C-statistic ± SEM^{b} | 0.7971 ± 0.0122 | | 0.8162 ± 0.0112 | 4.0 × 10⁻³ |
| −2 log-likehood ratio | 2708 | | 2650 | |
| AIC | 2720 | | 2666 | |
| NRI (versus clinical | | | 0.19 (0.13 to 0.25) | 6.5 × 10^{-10d} |
| model 1) (95% CI)^{c} | | | | |

Cox regression models were used to evaluate 10-year ESKD risk in the four combined cohorts (n = 745). The covariates selected by backward elimination for clinical model included HbA1c, baseline eGFR, ACR, and cohort indicator out of seven examined covariates (see Table 20). The covariates selected by backward elimination for combined clinical and AGP proteins included HbA1c, baseline eGFR, ACR, cohort indicator, and six AGP proteins.
^{a}The effects are shown as HRs (95% CIs) per one-quartile change of EFNA4 or EPHA2 (continuous variables).
^{b}Uno concordance statistics with two-sided P values. Null values for C-statistics are 0.5 and 3013 for AIC, respectively. P value versus model 1.
^{c}Risk categories for ESKD are 0%-4.9%, 5.0%-9.9%, 10.0%-19.9%, and ≥20% over 10 years.
^{d}P value versus model 1.

The six AGP proteins (EFNA4, EFNA5, EPHA2, EPHB2, EPHB6, and UNC5C) are members of two subfamilies comprising 34 known proteins, i.e., Ephrin ligands and receptors, and Netrin ligands and receptors. FIG. 4C lists these proteins and shows known binding preferences (44, 45). Of these 34 proteins, 14 (indicated with asterisks) were measured by SOMAscan and six (43%; indicated with arrows) were strongly associated with the development of ESKD.

Association of Severity of Kidney Structural Lesions with Circulating AGP Proteins but not with Expression of AGP Genes and Exemplar miRNAs in Kidney Biopsy Specimens

Table 10 shows the clinical characteristics of the 105 Pima Indian subjects who underwent research kidney biopsies and were included in this study.

Kidney Structural Lesions and Circulating Levels of AGP Proteins

Serum AGP protein concentrations correlated positively with VvMes, GBM thickness, and glomerular filtration surface area density (Table 15). Among the six AGP proteins, circulating EPHA2 had the strongest correlation with all structural lesions. Circulating EPHB6 and UNC5C levels also correlated significantly with most lesions, whereas the other AGP proteins had much weaker or no association with VvInt, podocyte abnormalities, and % FB.

TABLE 15
correlation between baseline serum concentrations of 6 AGP proteins and structural lesions
observed in kidney biopsy specimens obtained from 105 subjects in the T2D validation
cohort. Biopsies were obtained, on average, 1 year later after baseline examination

| | | Structural lesions | | | | | | | |
|---|---|---|---|---|---|---|---|---|---|
| | | VvInt | VvMes | GBM | Sv | Podo | FPW | PD | % FE |
| | | Spearman Correlation Coefficient | | | | | | | |
| | | r | r | r | r | r | r | r | r |
| Serum | EFNA4 | 0.09 | 0.33** | 0.25* | 0.27** | −0.15 | 0.05 | 0.07 | −0.19 |
| AGP | EFNA5 | 0.17 | 0.35** | 0.31** | 0.23* | −0.17 | 0.09 | 0.07 | −0.18 |
| proteins | EPHA2 | 0.27** | 0.52*** | 0.37*** | 0.32** | 0.26** | 0.21* | 0.21* | 0.25** |
| | EPHB2 | 0.07 | 0.21* | 0.09 | 0.20* | −0.08 | −0.08 | 0.02 | 0.00 |
| | EPHB6 | 0.07 | 0.43*** | 0.27** | 0.35** | 0.25* | 0.04 | 0.09 | −0.18 |
| | UNC5C | 0.11 | 0.42*** | 0.22* | 0.25* | 0.20* | 0.17 | 0.25** | −0.15 |

P
*<0.05,
**<0.01,
***<0.001

Kidney Structural Lesions and Expression of AGP Genes in Kidney Tissue

The mRNA expression levels for the six AGP proteins were measured in glomerular isolates from 66 subjects and in tubular isolates from 47 subjects. Spearman correlations showed little or no correlation between these AGP transcript levels and structural lesions in the same tissue (Table 16). Likewise, there was little or no correlation between levels of the four exemplar miRNAs in 40 glomerular and 26 tubular isolates from these biopsy specimens and the structural lesions (FIG. 4C), except for a significant correlation between the expression of miR-1287-5p in tubules and VvMes.

TABLE 16
Correlation between transcript levels of genes encoding for
AGP proteins in glomerular (n = 66) and tubular (n =
47) compartments, and structural lesions in kidney biopsy
specimens obtained from the subjects in T2D validation cohort

| | | | Structural Lesions | | | | |
|---|---|---|---|---|---|---|---|
| | | | VvInt | VvMes | GBM | Sv | % FE |
| | | | Spearman Correlation Coefficients | | | | |
| | | mRNA | r | r | r | r | r |
| Tissue | Glo- | EFNA4 | 0.07 | −0.07 | −0.11 | 0.07 | −0.11 |
| AGP | mer- | EFNA5 | −0.13 | −0.13 | −0.06 | 0.27* | −0.09 |
| Transcript | ular | EPHA2 | 0.12 | 0.27* | 0.16 | −0.20 | −0.15 |
| level | | EPHB6 | 0.08 | −0.16 | 0.30* | 0.19 | 0.09 |
| (mRNA) | | UNC5C | −0.18 | −0.09 | −0.15 | 0.03 | 0.07 |
| | Tu- | EFNA4 | −0.05 | 0.00 | −0.07 | −0.03 | −0.13 |
| | bular | EFNA5 | 0.02 | 0.19 | 0.17 | 0.31* | 0.04 |
| | | EPHA2 | −0.07 | 0.14 | 0.09 | −0.09 | 0.29* |
| | | EPHB6 | −0.12 | 0.17 | 0.11 | −0.19 | −0.03 |
| | | UNC5C | −0.22 | −0.04 | −0.05 | 0.05 | 0.19 |

P
*<0.05,
**<0.001

### Immunofluorescence Staining of AGP Proteins in Kidney Tissue

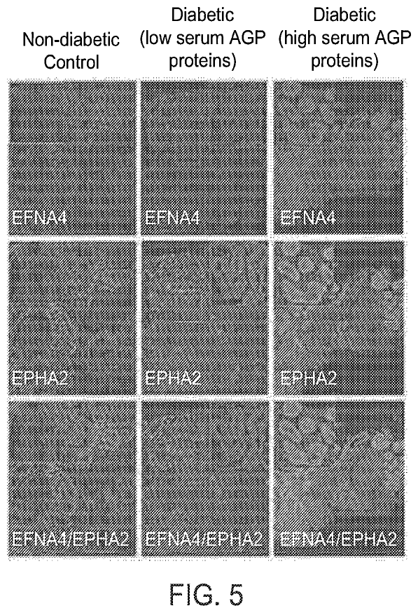

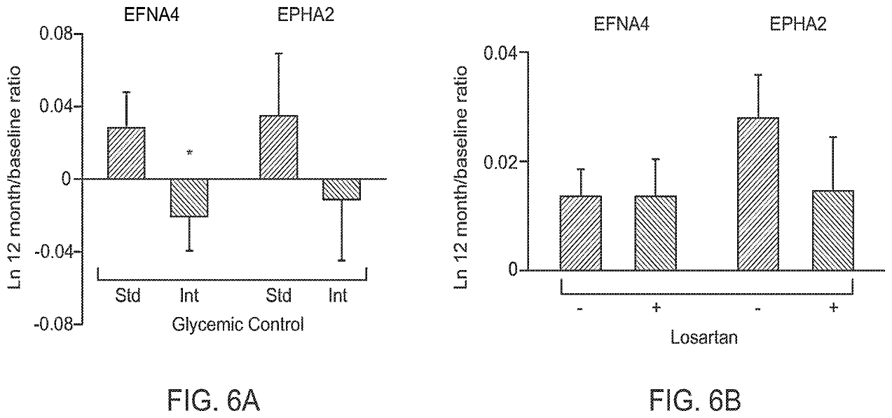

The kidney distribution of AGP proteins was assessed by immunofluorescence microscopy for EPHA2 and EFNA4 (a ligand of EPHA2) (46) in two subjects from the Pima cohort with low or high serum AGP proteins and one White subject who was an age-matched, nondiabetic control. Both EPHA2 and EFNA4 were primarily expressed in proximal tubules, with minimal staining in glomeruli, distal tubules, interstitium, and vessels (FIG. 5). The proximal tubules from the biopsy specimen of the subject with high serum AGP proteins showed increased staining intensities for EPHA2 and EFNA4 than those from the subject with low serum AGP proteins. The staining intensities in control, nondiabetic tissue were comparable to the biopsy specimen from the subject with diabetes who had low serum AGP proteins. EPHA2 showed almost perfect colocalization with EFNA4. No significant podocyte staining was seen for either protein. Despite the absence of staining for EPHA2 and EFNA4 in glomeruli, both biopsy specimens showed significant mesangial expansion (12% and 27%, respectively), and this lesion was highly correlated with circulating AGP protein levels.

### Clinical Relevance of Circulating AGP Proteins

Circulating AGP Proteins and Risk of ESKD During 7, 10, and 15 Years of Follow-Up

The association between circulating AGP proteins and risk of progression to ESKD may vary according to duration of follow-up. To test this, HRs for time to onset of ESKD during 7, 10, and 15 year of follow-up were computed, after adjustment for relevant covariates, according to quartiles of baseline concentration of each of the six AGP proteins. As shown in Table 17, each of the circulating AGP proteins had the strongest association with risk of progression (highest HRs) to ESKD during the first 7 years of follow-up. The HRs declined slightly during longer follow-up. These findings indicate that circulating AGP proteins were associated with fast progression.

TABLE 17
Sensitivity analysis

| | Follow-Up Duration | | | | | |
|---|---|---|---|---|---|---|
| | 7-Years (n = 187 | | 10 years (n = 240 | | 15 years (n = 289 | |
| | ESKD cases) | | ESKD cases) | | ESKD cases) | |
| Proteins | HR | P | HR | P | HR | P |
| EFNA4 | 1.95 | 7.5 × 10⁻¹⁶ | 1.84 | 6.4 × 10⁻¹⁸ | 1.72 | 1.6 × 10⁻¹⁷ |
| EFNA5 | 1.55 | 7.1 × 10⁻⁹ | 1.45 | 9.7 × 10⁻⁹ | 1.42 | 1.4 × 10⁻⁹ |
| EPHA2 | 2.15 | 3.2 × 10⁻²⁰ | 1.99 | 4.5 × 10⁻²² | 1.86 | 1.1 × 10⁻²² |
| EPHB2 | 1.55 | 3.0 × 10⁻⁹ | 1.51 | 1.7 × 10⁻¹⁰ | 1.42 | 2.6 × 10⁻⁹ |
| EPHB6 | 1.83 | 9.3 × 10⁻¹⁴ | 1.78 | 6.1 × 10⁻¹⁶ | 1.68 | 9.0 × 10⁻¹⁶ |
| UNC5C | 1.90 | 1.8 × 10⁻¹⁵ | 1.73 | 3.4 × 10⁻¹⁵ | 1.70 | 2.6 × 10⁻¹⁷ |

Comparison of HRs for time to onset of ESKD during 7, 10, and 15 years of follow-up, according to baseline levels of AGP protein, in the four combined cohorts (n = 745). HRs are shown for time to onset of ESKD according to a one-quartile increase (continuous variable) in baseline concentration of the candidate AGP proteins. HR were computed for etiologic models adjusted for sex, duration of diabetes, systolic BP, HbA1c, and eGFR, with variable stratification by study cohort.

Circulating AGP Proteins as Predictors of ESKD Risk

A role of the baseline circulating AGP proteins as predictors of progression to ESKD during 10 years of follow-up were evaluated in all four study cohorts by Cox regression. The prognostic performance of two different models was evaluated using C-statistics, AIC, and NRI at 10 years (Table 14). The model with EFNA4, EPHA2, and clinical covariates was characterized by improved fit using the Uno concordance statistic and the AIC, when compared with only the clinical model. Similarly, the categoric NRI for the model with EFNA4 and EPHA2 showed significant improvements compared with the clinical model. These results demonstrated that measuring the levels of EFNA4 and EPHA2 in circulation may have value as predictors of progression to ESKD.

Effect of Improved Glycemic Control on Circulating AGP Proteins

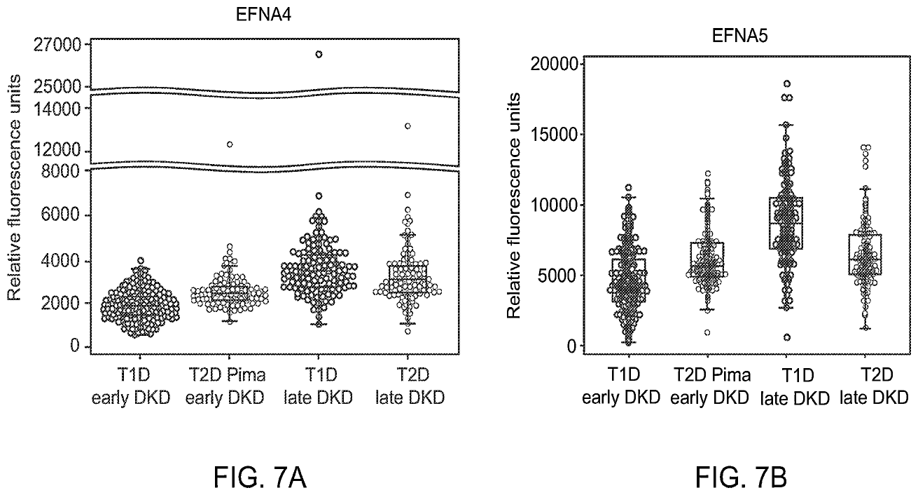

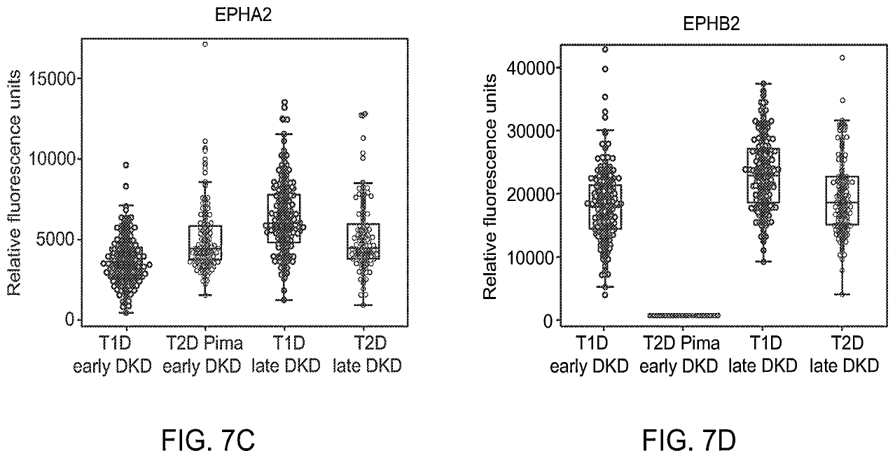

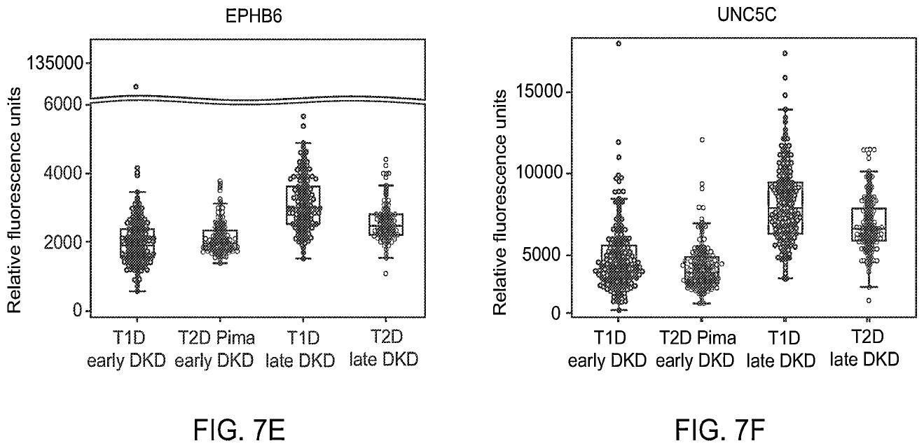

Circulating levels of AGP proteins might be surrogate end points for renoprotective therapies. To test this concept, we measured serum concentrations of EFNA4 and EPHA2 at baseline and at 12 months in a random sample of 200 participants with T2D in the ACCORD study. All baseline clinical characteristics and serum concentrations of EFNA4 and EPHA2 were similar according to glycemic treatment (see Table 18). In the standard glycemic control arm, mean HbA1c decreased from 8.2% at baseline to 7.7% at 12 months, whereas it decreased from 8.2% to 6.6% in the intensive glycemic control arm. In parallel with these changes, serum EFNA4 increased, on average, by 2.8% in the standard arm, whereas it decreased by 2.0% in the intensive arm (P=0.04 for the difference in EFNA4 levels between treatment arms at 12 months; FIG. 7A). A similar pattern was observed for EPHA2 (3.6% increase in the standard arm, 1.1% decrease in the intensive arm), although the difference between arms did not reach significance with this sample size (P=0.15; FIG. 7A).

TABLE 18
Baseline characteristics of ACCORD participants
in whom serum EFNA4 and EPHA2 were measured.

| | Standard | | Intensive | |
|---|---|---|---|---|
| | Glycemic | | Glycemic | |
| Baseline Characteristics | Treatment | | Treatment | |
| N | 100 | | 100 | |
| Female (n [%]) | 51 | (51) | 42 | (42) |
| Age (years) | 61.6 ± 6.1 | | 60.8 ± 5.5 | |
| Duration of DM (years) | 10.4 ± 6.3 | | 9.5 ± 7.3 | |
| BMI (kg/m²) | 33.5 ± 5.8 | | 31.8 ± 5.3 | |
| Systolic blood pressure (mmHg) | 132 ± 16 | | 134 ± 16 | |
| Diastolic blood pressure (mmHg) | 73 ± 9 | | 75 ± 10 | |
| Fasting plasma glucose (mg/dl) | 174 ± 59 | | 175 ± 52 | |
| HbA1c (%) | 8.2 ± 0.9 | | 8.2 ± 1.0 | |
| eGFR (ml/min/1.73 m²) | 94.1 ± 22 | | 93.9 ± 22 | |
| Previous CV event (n [%]) | 29 | (29) | 27 | (27) |
| EFNA4 (pg/ml) | 81.9 | (73, 96) | 82.1 | (73, 92) |
| EPHA2 (pg/ml) | 14.1 | (11, 18) | 13.9 | (11, 18) |

AGP proteins were measured by OLINK platform. Data are expressed as mean ± SD or median (first quartile, third quartile).
DM—Diabetes mellitus; BMI—Body mass index; CV—Cardiovascular.

TABLE 19
Comparison of AGP proteins measurements performed
by SOMAscan platform with measurements of
AGP proteins performed by OLINK platform.

| | Spearman correlation | | | | | |
|---|---|---|---|---|---|---|
| | coefficient (SOMAscan | | Cox model* for | | Cox model* for | |
| | vs. OLINK)^{¶} | | SOMAscan | | OLINK | |
| Gene | r | P | HR | P | HR | P |
| EFNA4 | 0.56 | 4.0 × 10⁻⁷ | 1.31 | 1.5 × 10⁻¹ | 1.91 | 1.3 × 10⁻³ |
| EPHA2 | 0.68 | 1.3 × 10⁻¹¹ | 1.57 | 1.8 × 10⁻² | 1.86 | 1.2 × 10⁻³ |
| EPHB6 | 0.60 | 2.8 × 10⁻⁸ | 1.93 | 1.0 × 10⁻³ | 2.12 | 2.2 × 10⁻⁴ |
| UNC5C | 0.68 | 4.1 × 10⁻¹¹ | 1.49 | 2.9 × 10⁻² | 1.33 | 1.3 × 10⁻¹ |

Correlation between AGP protein concentrations measured by SOMAscan and OLINK platforms in the same serum specimens in 75 subjects from Pima cohort.
*Univariate analysis for time to onset of ESKD during 10-year follow-up. HR (hazard ratio) was computed for a 1-quartile increase in concentration of AGP proteins. Pima cohort included 36 subjects who progressed to ESKD and 39 who did not.

TABLE 20
Assessment of importance of clinical covariates for time to onset of ESKD in combined 4 cohorts.
Results of Cox multivariable regression analyses with ACR absent and present in model.

| | Etiological model without ACR* | | | | Prognostic model with ACR** | | | |
|---|---|---|---|---|---|---|---|---|
| Parameter | HR | LCI | UCI | P | HR | LCI | UCI | P |
| Diastolic BP/10 (mm/Hg) | — | — | — | 3.3 × 10⁻¹ | — | — | — | 6.2 × 10⁻¹ |
| Sex (Female) | 0.74 | 0.57 | 0.97 | 3.1 × 10⁻² | — | — | — | 1.8 × 10⁻¹ |
| Duration of diabetes (year) | 0.98 | 0.97 | 1.00 | 3.6 × 10⁻² | — | — | — | 1.3 × 10⁻¹ |
| Systolic BP/10 (mm/Hg) | 1.10 | 1.02 | 1.19 | 1.1 × 10⁻² | — | — | — | 2.7 × 10⁻¹ |
| HbA1c (%) | 1.35 | 1.26 | 1.43 | 4.5 × 10⁻²⁰ | 1.25 | 1.16 | 1.34 | 1.8 × 10⁻⁹ |
| eGFR/10 (ml/min/1.73 m²) | 0.80 | 0.74 | 0.86 | 1.1 × 10⁻⁹ | 0.86 | 0.79 | 0.92 | 5.3 × 10⁻⁵ |
| log₂ACR (mg/g) | Not | — | — | — | 1.37 | 1.29 | 1.46 | 9.4 × 10⁻²³ |
| | included | | | | | | | |

HR—Hazard Ratios for time to onset of ESKD during a 10-year follow-up in combined 4 cohorts (n = 745).
*In etiological model, important covariates were selected by backward elimination from among all clinical covariates excluding ACR.
**In prognostic model, important covariates were selected by backward elimination from among all clinical covariates including ACR.

Effect of Treatment with Losartan and Circulating AGP Proteins

To examine whether treatment with angiotensin-converting enzyme inhibitors affected circulating AGP proteins, we examined levels of these proteins in 84 of the Pima Indian subjects who participated in a randomized clinical trial of losartan (29). Of these subjects, 47 were in the placebo arm and 37 were in the losartan arm. They received the study drug for at least 6 months before measurement of the AGP proteins. The levels of the six AGP proteins did not differ between the two subgroups. Both subgroups had second measurements of their AGP proteins 2-4 years later. Treatment with losartan during this period had no effect on the profile of AGP proteins (FIG. 7B). It is noteworthy that losartan treatment did not reduce the risk of kidney function decline in this 6-year trial (47).

### REFERENCES

1. Krolewski A S, Skupien J, Rossing P, Warram J H. Fast renal decline to end-stage renal disease: an unrecognized feature of nephropathy in diabetes. Kidney Int. 91(6):1300-1311 (2017)
2. Krolewski M, Eggers P W, Warram J H. Magnitude of end-stage renal disease in IDDM: a 35 year follow-up study. Kidney Int. 50:2041-2046 (1996)
3. LeCaire T J, Klein B E K, Howard K P, et al. Risk for end-stage renal disease over 25 years in the population-based WESDR cohort. Diabetes Care. 37:381-388 (2014)
4. Skupien J, Warram J H, Smiles A M, Stanton R C, Krolewski A S. Patterns of Estimated Glomerular Filtration Rate Decline Leading to End-Stage Renal Disease in Type 1 Diabetes. Diabetes Care. 39(12):2262-2269 (2016)
5. Collins A J, Foley R N, Herzog C, Chavers B, Gilbertson D, Ishani A U S renal data system 2010 annual report. Am J Kidney Dis. 57 (Suppl 1):A8-e1-526 (2011)
6. Thomas M C, Cooper M E, Zimmet P. Changing epidemiology of type 2 diabetes mellitus and associated chronic kidney disease. Nature Reviews Nephrology. 12:73-81 (2016)
7. Ghali W A et al. Comparison of 2 methods for calculating adjusted survival curves form proportional hazard models. JAMA. 286(12): 1494-1497 (2001)
8. Koye D N, Magliano D J, Reid C M, Jepson C, Feldman H I, Herman W H, Shaw J E. Risk of Progression of Nonalbuminuric CKD to End-Stage Kidney Disease in People With Diabetes: The CRIC (Chronic Renal Insufficiency Cohort) Study. Am J Kidney Dis. 72(5):653-661 (2018)
9. Yang W, Xie D, Anderson A H, Joffe M M, Greene T, Teal V, Hsu C Y, Fink J C, He J, Lash J P, Ojo A, Rahman M, Nessel L, Kusek J W, Feldman H I; CRIC Study Investigators. Association of kidney disease outcomes with risk factors for CKD: findings from the Chronic Renal Insufficiency Cohort (CRIC) study. Am J Kidney Dis. 63(2):236-43 (2014)
10. Ricardo A C, Yang W, Sha D, Appel U, Chen J, Krousel-Wood M, Manoharan A, Steigerwalt S, Wright J, Rahman M, Rosas S E, Saunders M, Sharma K, Daviglus M L, Lash J P; CRIC Investigators. Sex-Related Disparities in CKD Progression. J Am Soc Nephrol. 30(1):137-146 (2019)
11. Lundberg M, Eriksson A, Tran B, Assarsson E, Fredrikson S. Homogenous antibody-based proximity extension assays provide sensitive and specific detection of low-abundant proteins in human blood. Nucleic Acids Research. 39 (15):e102 (2011)
12. Assarsson E, Lundberg M, Holmquist G, Bjorkesten J, Thorsen S B, Ekman D, Eriksson A, Rennel Dickens E, Ohlsson S, Edfeldt G, Andersson A C, Lindstedt P, Stenvang J, Gullberg M, Fredriksson S. Homogenous 96-plex PEA immunoassay Exhibiting High Sensitivity, Specificity, and Excellent Scalability. PLOS One. 9(4):e95192 (2014)
13. Ghali W A et al. Comparison of 2 methods for calculating adjusted survival curves form proportional hazard models. JAMA. 286(12): 1494-1497 (2001)
14. Friedman J, Hastie T, Tibshirani R (2010). Regularization Paths for Generalized Linear Models via Coordinate Descent. J Stat Softw. 33(1): 1-22 (2010)
15. Simon N, Friedman J, Hastie T, Tibshirani R. Regularization Paths for Cox's Proportional Hazards Mod-el via Coordinate Descent. J Stat Softw. 39(5):1-13 (2011)
16. Graf E, Schmoor C, Sauerbrei, Schumacker M, Assessment and comparison of prognostic classification schemes for survival data. Stat Med. 18(17-18):2529-45 (1999)
17. Heagerty P J, Zheng Y. Survival model predictive accuracy and ROC curves. Biometrics. 61(1):92-105 (2005)
18. Pencina M J, D'Agostino R B Sr. Evaluating Discrimination of Risk Prediction Models

The C Statistic. JAMA. 8;314(10):1063-4 (2015)
19. Vickers A J, Elkin E B. Decision curve analysis: a novel method for evaluating prediction models. Med Decis Making. 26(6):565-74 (2006)
20. KDIGO: Definition, identification, and prediction of CDK progression. Kidney Int. (suppl 3):63-72 (2013)
21. Skupien J, Warram J H, Smiles A M, Niewczas M A, Gohda T, Pezzolesi M G, et al.: The early decline in renal function in patients with type 1 diabetes and proteinuria predicts the risk of end-stage renal disease. Kidney Int 82: 589-597, (2012)
22. Rosolowsky E T, Skupien J, Smiles A M, Niewczas M, Roshan B, Stanton R, et al.: Risk for ESRD in type 1 diabetes remains high despite reno-protection. J Am Soc Nephrol 22: 545-553, (2011)
23. Niewczas M A, Pavkov M E, Skupien J, Smiles A, Md Dom Z I, Wilson J M, et al.: A signature of circulating inflammatory proteins and development of end-stage renal disease in diabetes. Nat Med 25: 805-813, (2019)
24. Ihara K, Skupien J, Krolewski B, Md Dom Z I, O'Neil K, Satake E, et al.: A profile of multiple circulating tumor necrosis factor receptors associated with early progressive kidney decline in type 1 diabetes is similar to profiles in autoimmune disorders. Kidney Int 99: 725-736, (2021)
25. Skupien J, Warram J H, Smiles A, Galecki A, Stanton R C, Krolewski A S: Improved glycemic control and risk of ESRD in patients with type 1 diabetes and proteinuria. J Am Soc Nephrol 25: 2916-2925, (2014)
26. Md Dom Z I, Satake E, Skupien J, Krolewski B, O'Neil K, Willency J A, et al.: Circulating proteins protect against renal decline and progression to end-stage renal disease in diabetes: Results of global proteomics profiling. Sci Transl Med (2021, in press)
27. Nelson R G, Newman J M, Knowler W C, Sievers M L, Kunzelman C L, Pettitt D J, et al.: Incidence of end-stage renal disease in type 2 (non-insulin-dependent) diabetes mellitus in Pima Indians. Diabetologia 31: 730-736, (1988)
28. Nelson R G, Bennett P H, Beck G J, Tan M, Knowler W C, Mitch W E, et al.; Diabetic Renal Disease Study Group: Development and progression of renal disease in Pima Indians with non-insulin-dependent diabetes mellitus. N Engl J Med 335: 1636-1642, (1996)
29. Weil E J, Fufaa G, Jones L I, Lovato T, Lemley K V, Hanson R L, et al.: Effect of losartan on prevention and progression of early diabetic nephropathy in American Indians with type 2 diabetes. Diabetes 62: 3224-3231, (2013)
30. Gold L, Ayers D, Bertino J, Bock C, Bock A, Brody E N, et al.: Aptamer-based multiplexed proteomic technology for biomarker discovery. PLoS One 5: e15004, (2010)
31. Tuerk C, Gold L: Systematic evolution of ligands by exponential enrichment: RNA ligands to bacteriophage T4 DNA polymerase. Science 249: 505-510, (1990)
32. Ganz P, Heidecker B, Hveem K, Jonasson C, Kato S, Segal M R, et al.: Development and validation of a protein-based risk score for cardiovascular outcomes among patients with stable coronary heart disease. JAMA 315: 2532-2541, (2016)
33. Ngo D, Sinha S, Shen D, Kuhn E W, Keyes M J, Shi X, et al.: Aptamer-based proteomic profiling reveals novel candidate biomarkers and pathways in cardiovascular disease. Circulation 134: 270-285, (2016)
34. Williams S A, Murthy A C, DeLisle R K, Hyde C, Malarstig A, Ostroff R, et al.: Improving assessment of drug safety through proteomics: Early detection and mechanistic characterization of the unforeseen harmful effects of torcetrapib. Circulation 137: 999-1010, (2018)
35. Assarsson E, Lundberg M, Holmquist G, Bj€orkesten J, Thorsen S B, Ekman D, et al.: Homogenous 96-plex PEA immunoassay exhibiting high sensitivity, specificity, and excellent scalability. PLoS One 9: e95192, (2014)
36. Nair V, Komorowsky C V, Weil E J, Yee B, Hodgin J, Harder J L, et al.: A molecular morphometric approach to diabetic kidney disease can link structure to function and outcome. Kidney Int 93: 439-449, (2018)
37. Schmid H, Boucherot A, Yasuda Y, Henger A, Brunner B, Eichinger F, et al.; European Renal cDNA Bank (ERCB) Consortium: Modular activation of nuclear factor-kappaB transcriptional programs in human diabetic nephropathy. Diabetes 55: 2993-3003, (2006)
38. Cohen C D, Frach K, Schl€ondorff D, Kretzler M: Quantitative gene expression analysis in renal biopsies: A novel protocol for a high-throughput multicenter application. Kidney Int 61: 133-140, (2002)
39. Martini S, Nair V, Keller B J, Eichinger F, Hawkins J J, Randolph A, et al.; European Renal cDNA Bank; C-PROBE Cohort; CKDGen Consortium: Integrative biology identifies shared transcriptional networks in CKD. J Am Soc Nephrol 25: 2559-2572, (2014)
40. Gerstein H C, Miller M E, Byington R P, Goff D C Jr, Bigger J T, Buse J B, et al.; Action to Control Cardiovascular Risk in Diabetes Study Group: Effects of intensive glucose lowering in type 2 diabetes. N Engl J Med 358: 2545-2559, (2008)
41. CaramoriML, Kim Y, Goldfine A B, Moore J H, Rich S S, Mychaleckyj J C, et al.: Differential gene expression in diabetic nephropathy in individuals with type 1 diabetes. J Clin Endocrinol Metab 100: E876-E882, (2015)
42. Pipino C, Shah H, Prudente S, Di Pietro N, Zeng L, Park K, et al.: Association of the 1q25 diabetes-specific coronary heart disease locus with alterations of the g-glutamyl cycle and increased methylglyoxal levels in endothelial cells. Diabetes 69: 2206-2216, (2020)
43. Di Tomo P, Canali R, Ciavardelli D, Di Silvestre S, De Marco A, Giardinelli A, et al.: b-Carotene and lycopene affect endothelial response to TNF-α reducing nitro-oxidative stress and interaction with monocytes. Mol Nutr Food Res 56: 217-227, (2012)
44. Coulthard M G, Morgan M, Woodruff T M, Arumugam T V, Taylor S M, Carpenter T C, et al.: Eph/Ephrin signaling in injury and inflammation. Am J Pathol 181: 1493-1503, (2012)
45. Larrieu-Lahargue F, Thomas K R, Li D Y: Netrin ligands and receptors: Lessons from neurons to the endothelium. Trends Cardiovasc Med 22: 44-47, (2012)
46. Pasquale E B: The Eph family of receptors. Curr Opin Cell Biol 9: 608-615, (1997)
47. Tanamas S K, Saulnier P J, Fufaa G D, Wheelock K M, Weil E J, Hanson R L, et al.: Long-term effect of Losartan on kidney disease in American Indians with type 2 diabetes: A follow-up analysis of a randomized clinical trial. Diabetes Care 39: 2004-2010, (20160

1. A method for determining whether a human subject has or is at risk of developing renal decline, said method comprising
detecting a level of at least one renal decline marker in a biological sample from the human subject,
wherein the human subject has or is at risk of developing renal decline if the level of the at least one renal decline marker correlates with a known standard for a human subject who has or is at risk of developing renal decline, or
wherein the human subject does not have or is not at risk of developing renal decline if the level of the at least one renal decline marker correlates with a known standard for a human subject who does not have or is not at risk of developing renal decline.

2. The method of claim 1, further comprising
comparing the level of the at least one renal decline marker from the biological sample from the human subject to a non-renal-decliner control level of the at least one renal decline marker; and
determining whether the level of the at least one renal decline marker from the biological sample is equal to or higher than the level of the at least one renal decline marker of a non-renal-decliner control, wherein a higher level of the at least one renal decline marker from the biological sample from the human subject relative to the level of the at least one renal decline marker from the non-renal-decliner control indicates that the human subject has or is at risk of developing renal decline; and/or wherein the method further comprises comparing the level of the at least one renal decline marker from the biological sample from the human subject to a normoalbuminuric control level of the at least one renal decline marker; and
determining whether the level of the at least one renal decline marker from the biological sample is equal to or higher than the level of the at least one renal decline marker of a normoalbuminuric control, wherein a higher level of the at least one renal decline marker from the biological sample from the human subject relative to the level of the at least one renal decline marker from the normoalbuminuric control indicates that the human subject has or is at risk of developing renal decline; and/or wherein the method further comprises contacting the biological sample from the human subject with a device for measuring the protein level of the at least one renal decline marker; and/or wherein the device is capable of performing an immunoassay, a mass spectrometry analysis, a Slow Off-rate Modified Aptamer (SOMA)scan platform analysis, liquid chromatography (LC) fractionation, Mesoscale platform, electrochemiluminescence detection, or an OLINK Proximity Extension Assay based proteomic platform analysis.

3-5. (canceled)

6. The method of claim 1, wherein the at least one renal decline marker is a protein and includes at least one of TNF-R1, TNF-R2, CD27, LTBR, TNF-RSF6B, TNF-RSF10A, TNF-RSF4, EDA2R, RELT, CD160, IL-1RT1, DLL1, LAYN, MMP7, NBL1, PI3, WFDC2, EFNA4, EPHA2, GFR-alpha-1, and KIM1.

7. The method of claim 1, further comprising
measuring an estimated glomerular function rate (eGFR) slope of the human subject and
determining whether the eGFR slope of the human subject indicates that the human subject has or is at risk of developing renal decline.

8. The method of claim 7, wherein an eGFR slope of at least <−5 ml/min/year indicates that the human subject has or is at risk of developing renal decline; and/or wherein an eGFR slope of at least <−10 mL/min/year indicates that the human subject has or is at risk of developing renal decline; and/or wherein an eGFR slope of at least <−15 mL/min/year indicates that the human subject has or is at risk of developing renal decline; and/or wherein the renal decline is (i) a very fast renal decline comprising an estimated time to reach onset of end-stage renal disease (ESRD) of 2-6 years, (ii) a fast renal decline comprising an estimated time to reach onset of end-stage renal disease (ESRD) of 6-10 years, or (iii) a moderate renal decline comprising an estimated time to reach onset of end-stage renal disease (ESRD) of 10-20 years.

9-11. (canceled)

12. The method of claim 1, further comprising
measuring a urine albumin to creatinine ratio (ACR) of the human subject, and
determining whether the ACR of the human subject indicates that the human subject has micro-albuminuria or macro-albuminuria; and/or wherein the human subject has early progressive renal decline or late progressive renal decline; and/or wherein the human subject has type I diabetes (T1D) or type 2 diabetes (T2D); and/or wherein the renal decline is early renal decline; and/or wherein the renal decline is late renal decline; and/or wherein the method further comprises treating the human subject having or at risk of developing renal decline.

13-17. (canceled)

18. A method for determining whether a human subject has or is at risk of developing end-stage renal disease (ESRD), said method comprising
detecting the level of at least one ESRD marker in a biological sample from the human subject,
wherein the human subject has or is at risk of developing ESRD if the level of the at least one ESRD marker correlates with a known standard for a human subject who has or is at risk of developing ESRD, or
wherein the human subject does not have or is not at risk of developing ESRD if the level of the at least one ESRD marker correlates with a known standard for a human subject who does not have or is not at risk of developing ESRD.

19. The method of claim 18, further comprising
comparing the level of the at least one ESRD marker from the biological sample from the human subject to a non-ESRD control level of the at least one ESRD marker; and
determining whether the level of the at least one ESRD marker from the biological sample is equal to or higher than the level of the at least one ESRD marker of a non-ESRD control, wherein a higher level of the at least one ESRD marker from the biological sample from the human subject relative to the level of the at least one ESRD marker from the non-ESRD control indicates that the human subject has or is at risk of developing ESRD; and/or further comprising comparing the level of the at least one ESRD marker from the biological sample from the human subject to a normoalbuminuric control level of the at least one ESRD marker; and
determining whether the level of the at least one ESRD marker from the biological sample is equal to or higher than the level of the at least one ESRD marker of a normoalbuminuric control, wherein a higher level of the at least one ESRD marker from the biological sample from the human subject relative to the level from the normoalbuminuric control indicates that the human subject has or is at risk of developing ESRD; and/or further comprising contacting the biological sample from the human subject with a device for measuring the protein level of the at least one ESRD marker; and/or wherein the device is useful for performing an immunoassay, a mass spectrometry analysis, a Slow Off-rate Modified Aptamer (SOMA)scan platform analysis, or an OLINK Proximity Extension Assay based proteomic platform analysis.

20-22. (canceled)

23. The method of claim 18, wherein the at least one ESRD marker includes at least one of TNF-R1, TNF-R2, CD27, TNF-RSF6B, FR-alpha, TNF-RSF10A, TNF-RSF14, EDA2R, RELT, CD160, IL-1RT1, DLL1, LAYN, MMP7, NBL1, WFDC2, EFNA4, EPHA2, GFR-alpha-1, and KIM1.

24. The method of claim 18, further comprising
measuring a urine albumin to creatinine ratio (ACR) of the human subject, and
determining whether the ACR of the human subject indicates that the human subject has micro-albuminuria or macro-albuminuria; and/or wherein the method further comprises determining a baseline renal function of the human subject.

25. (canceled)

26. The method of claim 18, further comprising
measuring an estimated glomerular function rate (eGFR) slope of the human subject and
determining a time to onset of ESRD for the human subject using the level of the at least one ESRD marker and/or the eGFR slope of the human subject.

27. The method of claim 26, wherein an eGFR slope of at least <−15 ml/min/year indicates that the time to onset of ESRD for the human subject is 2-6 years; an eGFR slope of between <−15 ml/min/year and <−10 ml/min/year indicates that the time to onset of ESRD for the human subject is 6-10 years; and an eGFR slope of between <−10 ml/min/year and <−5 ml/min/year indicates that the time to onset of ESRD for the human subject is 10-20 years.

28-30. (canceled)

31. The method of claim 18, wherein the human subject has early progressive renal decline, late progressive renal decline, type I diabetes (T1D), or type 2 diabetes (T2D); and/or wherein the method further comprises treating the human subject having or at risk of developing ESRD.

32-34. (canceled)

35. A method of monitoring the progression of renal decline or end-stage renal disease (ESRD) in a human subject, said method comprising
contacting a biological sample from the human subject with a device for assaying the protein level of at least one renal decline marker or at least one ESRD marker selected from one or more of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22,
measuring the amount of the at least one renal decline marker or the at least one ESRD marker in the biological sample as compared to a control sample, wherein an increased or a decreased level of the at least one renal decline marker or the at least one ESRD marker relative to the control sample indicates progression of renal decline or ESRD in the human subject and/or wherein the device is useful for performing an immunoassay, a mass spectrometry analysis, a Slow Off-rate Modified Aptamer (SOMA)scan platform analysis, liquid chromatography (LC) fractionation, Mesoscale platform, electrochemiluminescence detection, or an OLINK Proximity Extension Assay based proteomic platform analysis.

36. (canceled)

37. A method of monitoring efficacy of a renal decline or an end-stage renal disease (ESRD) treatment regimen in a human subject, the method comprising:
obtaining a first biological sample from the human subject at a first time point;
administering the treatment regimen to the human subject;
obtaining a second biological sample from the human subject at a second time point;
detecting at least one protein level selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22 in the first sample; and
detecting the at least one protein level in the second sample.

38. The method of claim 37, further comprising changing or repeating the treatment regimen when the at least one protein level for the first sample is the same as the at least one protein level for the second sample; and/or wherein the method further comprises discontinuing the treatment regimen when the at least one protein level of the second sample is the same as the level corresponding to a healthy individual; and/or wherein the detecting is performed with a device for measuring the level of the at least one ESRD marker; and/or wherein the device is useful for performing an immunoassay, a mass spectrometry analysis, a Slow Off-rate Modified Aptamer (SOMA) scan platform analysis, liquid chromatography (LC) fractionation, Mesoscale platform, electrochemiluminescence detection, or an OLINK Proximity Extension Assay based proteomic platform analysis; and/or wherein the biological sample is selected from the group consisting of a blood sample, a plasma sample, a serum sample, a saliva sample, and a urine sample.

39-42. (canceled)

43. A method of determining the approximate risk of renal decline (RD) or end-stage renal disease (ESRD) in a human subject, the method comprising:
a) detecting, in a biological sample from the human subject, the level of at least two RD-associated proteins of a biomarker panel or at least two ESRD-associated proteins of a biomarker panel, wherein the biomarker panel comprises at least two proteins selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KIM1, TNFRSF11A, CLM1, TNFRSF12A, TRAIL-R2, RGMB, DKK4, TFF3, CRELD2, CADM3, and ADAM22; and
b) determining the approximate risk of renal decline (RD) for the human subject, and/or the risk of end-stage renal disease (ESRD) of the human subject.

44. The method of claim 43, wherein the determining comprises employing an algorithm to generate a renal decline (RD) risk score or end-stage renal disease (ESRD) risk score, wherein the algorithm performs operations comprising:
i) adjusting each RD-associated protein level or each ESRD-associated protein level by a predetermined coefficient to generate an adjusted value, and
ii) adding or multiplying the adjusted value together, thereby generating the RD risk score or the ESRD risk score; and/or wherein the level of the at least two RD associated proteins or the at least two ESRD associated proteins is assessed by an immunoassay, a Slow Off-rate Modified Aptamer (SOMA) scan platform, liquid chromatography (LC) fractionation, Mesoscale platform, electrochemiluminescence detection, or an OLINK Proximity Extension Assay based proteomic platform; and/or wherein the algorithm performs operations further comprising:
i) determining an albumin-to-creatinine ratio (ACR) for the human subject;
ii) adjusting the ACR by a predetermined coefficient to generate an adjusted ACR value; and
iii) adding or multiplying the adjusted values together, thereby generating the RD risk score or the ESRD risk score; and/or wherein the algorithm performs operations further comprising:
i) determining a systolic blood pressure (SBP) for the human subject;
ii) adjusting the SBP by a predetermined coefficient to a generate an adjusted SBP value; and
iii) adding or multiplying the adjusted values together, thereby generating the RD risk score or the ESRD risk score; and/or wherein the algorithm performs operations further comprising:
i) determining an estimated glomerular filtration rate (eGFR) for the human subject;
ii) adjusting the eGFR by a predetermined coefficient to a generate an adjusted eGFR value; and
iii) adding or multiplying the adjusted values together, thereby generating the RD risk score or the ESRD risk score; and/or wherein the method further comprises c) generating a report that provides the RD risk score and/or the ESRD risk score; and/or wherein the biological sample is selected from the group consisting of a blood sample, a plasma sample, a serum sample, a saliva sample and a urine sample; and/or wherein the human subject is a non-diabetic patient.

45-51. (canceled)

52. A protein array comprising at least two biomarkers useful for diagnosing, predicting, and/or monitoring of renal decline or end-stage renal disease in a sample of a human subject, wherein the biomarkers are selected from the group consisting of TNF-R1, TNF-R2, CD27, LTBR, TNF-RSF6B, FR-alpha, TNF-RSF10A, TNF-RSF14, TNF-RSF4, EDA2R, RELT, CD160, IL-1RT1, DLL1, LAYN, MMP7, NBL1, PI3, WFDC2, EFNA4, EPHA2, GFR-alpha-1, KM, TNFRSF11A, CLM1, TNFRSF12A, TRAIL-R2, RGMB, DKK4, TFF3, CRELD2, CADM3, and ADAM22, or fragments, or variants thereof.

53. A test panel comprising the protein array of claim 52; and/or wherein the test panel is in a kit or assay device.

54. (canceled)


SEQUENCE LISTING (33 sequences)

SEQ ID NO: 1 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 2 - protein, 450 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLP

SEQ ID NO: 3 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 4 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 5 - protein, 300 aa, Homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 6 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 7 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAREKIQDLLVDSGKFIYLEDGTGSAVSLE

SEQ ID NO: 8 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 9 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 10 - protein, 297 aa, Homo sapiens
MDCQENEYWDQWGRCVTCQRCGPGQELSKDCGYGEGGDAYCTACPPRRYKSSWGHHRCQSCITCAVINRVQKVNCTATSNAVCGDCLPRFYRKTRIGGLQDQECIPCTKQTPTSEVQCAFQLSLVEADTPTVPPQEATLVALVSSLLVVFTLAFLGLFFLYCKQFFNRHCQRGGLLQFEADKTAKEESLFPVPPSKETSAESQVSENIFQTQPLNPILEDDCSSTSGFPTQESFTMASCTSESHSHWVHSPIECTELDLQKFSSSASYTGAETLGGNTVESTGDRLELNVPFEVPSP

SEQ ID NO: 11 - protein, 430 aa, Homo sapiens
MKPSLLCRPLSCFLMLLPWPLATLTSTTLWQCPPGEEPDLDPGQGTLCRPCPPGTFSAAWGSSPCQPHARCSLWRRLEAQVGMATRDTLCGDCWPGWFGPWGVPRVPCQPCSWAPLGTHGCDEWGRRARRGVEVAAGASSGGETRQPGNGTRAGGPEETAAQYAVIAIVPVFCLMGLLGILVCNLLKRKGYHCTAHKEVGPGPGGGGSGINPAYRTEDANEDTIGVLVRLITEKKENAAALEELLKEYHSKQLVQTSHRPVSKLPPAPPNVPHICPHRHHLHTVQGLASLSGPCCSRCSQKKWPEVLLSPEAVAATTPVPSLLPNPTRVPKAGAKAGRQGEITILSVGRFRVARIPEQRTSSMVSEVKTITEAGPSWGDLPDSPQPGLPPEQQALLGSGGSRTKWLKPPAENKAEENRYVVRLSESNLVI

SEQ ID NO: 12 - protein, 181 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 13 - protein, 569 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 14 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 15 - protein, 382 aa, Homo sapiens
MRPGTALQAVLLAVLLVGLRAATGRLLSASDLDLRGGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 16 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 17 - protein, 181 aa, Homo sapiens
MMLRVLVGAVLPAMLLAAPPPINKLALFPDKSAWCEAKNITQIVGHSGCEAKSIQNRACLGQCFSYSVPNTFPQSTESLVHCDSCMPAQSMWEIVTLECPGHEEVPRVDKLVEKILHCSCQACGKEPSHEGLSVYVQGEDGPGSQPGTHPHPHPHPHPGGQTPEPEDPPGAPHTEEEGAED

SEQ ID NO: 18 - protein, 124 aa, Homo sapiens
MPACRLGPLAAALLLSLLLFGFTLVSGTGAEKTGVCPELQADQNCTQECVSDSECADNLKCCSAGCATFCSLPNDKEGSCPQVNINFPQLGLCRDQCQVDSQCPGQMKCCRNGCGKVSCVTPNF

SEQ ID NO: 19 - protein, 201 aa, Homo sapiens
MRLLPLLRTVLWAAFLGSPLRGGSSLRHVVYWNSSNPRLLRGDAVVELGLNDYLDIVCPHYEGPGPPEGPETFALYMVDWPGYESCQAEGPRAYKRWVCSLPFGHVQFSEKIQRFTPFSLGFEFLPGETYYYISVPTPESSGQCLRLQVSVCCKERKSESAHPVGSPGESGTSGWRGGDTPSPLCLLLLLLLLILRLLRIL

SEQ ID NO: 20 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 21 - protein, 465 aa, Homo sapiens
MFLATLYFALPLLDLLLSAEVSGGDRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 22 - protein, 364 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 23 - protein, 117 aa, Homo sapiens
MRASSFLIVVVFLIAGTLVLEAAVTGVPVKGQDTVKGRVPFNGQDPVKGQVSVKGQDKVKAQEPVKGPVSTKPGSCPIILIRCAMLNPPNRCLKDTDCPGIKKCCEGSCGMACFVPQ

SEQ ID NO: 24 - protein, 616 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 25 - protein, 290 aa, Homo sapiens
MPLLTLYLLLFWLSGYSIVTQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPAPVTQEETSSSPTLTGHHLDNRHKLLKLSVLLPLIFTILLLLLVAASLLAWRMMKYQQKAAGMSPEQVLQPLEGDLCYADLTLQLAGTSPQKATTKLSSAQVDQVEVEYVTMASLPKEDISYASLTLGAEDQEPTYCNMGHLSSHLPGRGPEEPTEYSTISRP

SEQ ID NO: 26 - protein, 129 aa, Homo sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 27 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 28 - protein, 437 aa, Homo sapiens
MGLRAAPSSAAAAAAEVEQRRSPGLCPPPLELLLLLLFSLGLLHAGDCQQPAQCRIQKCTTDFVSLTSHLNSAVDGFDSEFCKALRAYAGCTQRTSKACRGNLVYHSAVLGISDLMSQRNCSKDGPTSSTNPEVTHDPCNYHSHAGAREHRRGDQNPPSYLFCGLFGDPHLRTFKDNFQTCKVEGAWPLIDNNYLSVQVTNVPVVPGSSATATNKITIIFKAHHECTDQKVYQAVTDDLPAAFVDGTTSGGDSDAKSLRIVERESGHYVEMHARYIGTTVFVRQVGRYLTLAIRMPEDLAMSYEESQDLQLCVNGCPLSERIDDGQGQVSAILGHSLPRTSLVQAWPGYTLETANTQCHEKMPVKDIYFQSCVFDLLTTGDANFTAAAHSALEDVEALHPRKERWHIFPSSGNGTPRGGSDLSVSLGLTCLILIVFL

SEQ ID NO: 29 - protein, 224 aa, Homo sapiens
MVAAVLLGLSWLCSPLGALVLDFNNIRSSADLHGARKGSQCLSDTDCNTRKFCLQPRDEKPFCATCRGLRRRCQRDAMCCPGTLCVNDVCTTMEDATPILERQLDEQDGTHAEGTTGHPVQENQPKRKPSIKKSQGRKGQEGESCLRTFDCGPGLCCARHFWTKICKPVLLEGQVCSRRGHKDTAQAPEIFQRCDCGPGLLCRSQLTSNRQHARLRVCQKIEKL

SEQ ID NO: 30 - protein, 94 aa, Homo sapiens
MKRVLSCVPEPTVVMAARALCMLGLVLALLSSSSAEEYVGLSANQCAVPAKDRVDCGYPHVTPKECNNRGCCFDSRIPGVPWCFKPLQEAECTF

SEQ ID NO: 31 - protein, 353 aa, Homo sapiens
MRLPRRAALGLLPLLLLLPPAPEAAKKPTPCHRCRGLVDKFNQGMVDTAKKNFGGGNTAWEEKTLSKYESSEIRLLEILEGLCESSDFECNQMLEAQEEHLEAWWLQLKSEYPDLFEWFCVKTLKVCCSPGTYGPDCLACQGGSQRPCSGNGHCSGDGSRQGDGSCRCHMGYQGPLCTDCMDGYFSSLRNETHSICTACDESCKTCSGLTNRDCGECEVGWVLDEGACVDVDECAAEPPPCSAAQFCKNANGSYTCEECDSSCVGCTGEGPGNCKECISGYAREHGQCADVDECSLAEKTCVRKNENCYNTPGSYVCVCPDGFEETEDACVPPAEAEATEGESPTQLPSREDL

SEQ ID NO: 32 - protein, 398 aa, Homo sapiens
MGAPAASLLLLLLLFACCWAPGGANLSQDDSQPWTSDETVVAGGTVVLKCQVKDHEDSSLQWSNPAQQTLYFGEKRALRDNRIQLVTSTPHELSISISNVALADEGEYTCSIFTMPVRTAKSLVTVLGIPQKPIITGYKSSLREKDTATLNCQSSGSKPAARLTWRKGDQELHGEPTRIQEDPNGKTFTVSSSVTFQVTREDDGASIVCSVNHESLKGADRSTSQRIEVLYTPTAMIRPDPPHPREGQKLLLHCEGRGNPVPQQYLWEKEGSVPPLKMTQESALIFPFLNKSDSGTYGCTATSNMGSYKAYYTLNVNDPSPVPSSSSTYHAIIGGIVAFIVFLLLIMLIFLGHYLIRHKGTYLTHEAKGSDDAPDADTAIINAEGGQSGGDDKKEYFI

SEQ ID NO: 33 - protein, 906 aa, Homo sapiens
MQAAVAVSVPFLLLCVLGTCPPARCGQAGDASLMELEKRKENRFVERQSIVPLRLIYRSGGEDESRHDALDTRVRGDLGGPQLTHVDQASFQVDAFGTSFILDVVLNHDLLSSEYIERHIEHGGKTVEVKGGEHCYYQGHIRGNPDSFVALSTCHGLHGMFYDGNHTYLIEPEENDTTQEDFHFHSVYKSRLFEFSLDDLPSEFQQVNITPSKFILKPRPKRSKRQLRRYPRNVEEETKYIELMIVNDHLMFKKHRLSVVHTNTYAKSVVNMADLIYKDQLKTRIVLVAMETWATDNKFAISENPLITLREFMKYRRDFIKEKSDAVHLFSGSQFESSRSGAAYIGGICSLLKGGGVNEFGKTDLMAVTLAQSLAHNIGIISDKRKLASGECKCEDTWSGCIMGDTGYYLPKKFTQCNIEEYHDFLNSGGGACLFNKPSKLLDPPECGNGFIETGEECDCGTPAECVLEGAECCKKCTLTQDSQCSDGLCCKKCKFQPMGTVCREAVNDCDIRETCSGNSSQCAPNIHKMDGYSCDGVQGICFGGRCKTRDRQCKYIWGQKVTASDKYCYEKLNIEGTEKGNCGKDKDTWIQCNKRDVLCGYLLCTNIGNIPRLGELDGEITSTLVVQQGRTLNCSGGHVKLEEDVDLGYVEDGTPCGPQMMCLEHRCLPVASFNFSTCLSSKEGTICSGNGVCSNELKCVCNRHWIGSDCNTYFPHNDDAKTGITLSGNGVAGTNIIIGIIAGTILVLALILGITAWGYKNYREQRQLPQGDYVKKPGDGDSFYSDIPPGVSTNSASSSKKRSNGLSHSWSERIPDTKHISDICENGRPRSNSWQGNLGGNKKKIRGKRFRPRSNSTETLSPAKSPSSSTGSIASSRKYPYPMPPLPDEDKKVNRQSARLWETSI